(19) United States
(12) Patent Application Publication
(10) Pub. No.: US 2018/0125796 A1
(43) Pub. Date: May 10, 2018

(54) TREATMENT OF MYCOBACTERIAL INFECTION

(71) Applicant: Oxford University Innovation Limited, Oxford, Oxfordshire (GB)

(72) Inventors: Frances Mary Platt, Oxford (Oxfordshire) (GB); Nick Platt, Oxford (Oxfordshire) (GB); Paul Fineran, Oxford (Oxfordshire) (GB); Edith Sim, Oxford (Oxfordshire) (GB); Emyr Lloyd-Evans, Oxford (Oxfordshire) (GB); Nathan Lack, Oxford (Oxfordshire) (GB)

(21) Appl. No.: 15/541,809

(22) PCT Filed: Jan. 7, 2016
(86) PCT No.: PCT/GB2016/050029

§ 371 Date: Jul. 6, 2017

(30) Foreign Application Priority Data
Jan. 7, 2015 (GB) 1500207.4

(51) Int. Cl. A61K 31/12; A61P 31/06; A61K 31/445; A61K 31/4025

(52) U.S. Cl. CPC A61K 31/12; A61K 31/4025; A61K 31/445; A61P 31/06

(57) ABSTRACT

The invention provides a combination comprising a SERCA antagonist and an inhibitor of glycosphingolipid biosynthesis, for use in the treatment of an infection by a pathogenic mycobacterium. The combination may for instance be used to treat tuberculosis.

### CROSS-REFERENCE TO RELATED APPLICATION

This application is the National Stage of International Application No. PCT/GB2016/050029, filed 7 Jan. 2016, which claims the benefit of and priority to GB Application No. 1500207.4, having the title “Treatment of Mycobacterial Infection,” filed on 7 Jan. 2015, the entire disclosures of which are incorporated by reference in their entireties as if fully set forth herein.

### GOVERNMENT SUPPORT

This invention was made with government funds under Grant/Contract No. 4R21AI102166-02 awarded by NIH/NIAID. The US Government has rights in this invention.

### FIELD OF THE INVENTION

The present invention relates to the treatment of an infection by a pathogenic mycobacterium, and in particular to the treatment of tuberculosis.

### BACKGROUND TO THE INVENTION

Approximately one-third of the world's population is infected with Mycobacterium tuberculosis (Mtb), the causative agent of human tuberculosis (TB). TB causes around 2 million deaths per year, a significant number of which are immune-compromised individuals (Gray, J. M. & Cohn, D. L. Tuberculosis and HIV coinfection. Seminars in respiratory and critical care medicine 34, 32-43, doi:10.1055/s-0032-1333469; 2013). The only approved vaccine, Bacillus Calmette-Guerin (BCG) has limited efficacy (Evans, T. G., Brennan, M. J., Barker, L. & Thole, J. Preventive vaccines for tuberculosis. Vaccine 31 Suppl 2, B223-226, doi:10.1016/j.vaccine.2012.11.081; 2013) and the emergence of antibiotic-resistant TB strains has led to a reduction in available therapeutic options. Consequently the development of new TB therapies is of paramount importance.

Mtb (and the model organism BCG) have the unusual ability to invade, persist and replicate within cells of the innate immune system, particularly alveolar macrophages (Russell, D. G., Cardona, P. J., Kim, M. J., Allain, S. & Altare, F. Foamy macrophages and the progression of the human tuberculosis granuloma. Nat Immunol 10, 943-948, doi:10.1038/ni.1781; 2009). Mtb-infected cells develop a characteristic cholesterol-laden foamy cell phenotype (Peyron, P. et al. Foamy macrophages from tuberculous patients' granulomas constitute a nutrient-rich reservoir for M. tuberculosis persistence. PLoS Pathog 4, e1000204, doi:10.1371/journal.ppat.1000204; 2008) and have evolved the unusual ability to metabolise host cholesterol as a carbon source (Lack, N. A. et al. Characterization of a carbon-carbon hydrolase from Mycobacterium tuberculosis involved in cholesterol metabolism. J Biol Chem 285, 434-443, doi:10.1074/jbc.M109.058081; 2010; Pandey, A. K. & Sassetti, C. M. Mycobacterial persistence requires the utilization of host cholesterol. Proc Natl Acad Sci USA 105, 4376-4380, doi:10.1073/pnas.0711159105; 2008; Thomas, S. T., VanderVen, B. C., Sherman, D. R., Russell, D. G. & Sampson, N. S. Pathway profiling in Mycobacterium tuberculosis: elucidation of cholesterol-derived catabolite and enzymes that catalyze its metabolism. J Biol Chem 286, 43668-43678, doi:10.1074/jbc.M111.313643; 2011). Non-pathogenic mycobacteria, including Mycobacterium smegmatis, bind host cell-surface receptors and are ingested into phagosomes that subsequently mature and fuse with lysosomes, leading to destruction of the mycobacterium. In contrast, pathogenic mycobacteria are not delivered to the lysosome due to the mycobacterium inhibiting phagosome-lysosome fusion. Multiple mechanisms have been proposed to explain this, including phagosome maturation arrest (Jayachandran, R. et al. Survival of mycobacteria in macrophages is mediated by coronin 1-dependent activation of calcineurin. Cell 130, 37-50, doi:10.1016/j.ce11.2007.04.043; 2007), defective acidification (Sturgill-Koszycki, S. et al. Lack of acidification in Mycobacterium phagosomes produced by exclusion of the vesicular proton-ATPase. Science 263, 678-681; 1994) and inhibition of phosphatidylinositol-dependent trafficking pathways (Vergne, I., Chua, J. & Deretic, V. Tuberculosis toxin blocking phagosome maturation inhibits a novel Ca²⁺/calmodulin-PI3K hVPS34 cascade. J Exp Med 198, 653-659, doi:10.1084/jem.20030527; 2003; Vergne, I., Chua, J. & Deretic, V. Mycobacterium tuberculosis phagosome maturation arrest: selective targeting of PI3P-dependent membrane trafficking. Traffic 4, 600-606; 2003). Calcium ions (Ca²⁺) have also been implicated, as phagosome-lysosome fusion is stimulated by an elevation of cytosolic Ca²⁺ (Majeed, M., Perskvist, N., Ernst, J. D., Orselius, K. & Stendahl, O. Roles of calcium and annexins in phagocytosis and elimination of an attenuated strain of Mycobacterium tuberculosis in human neutrophils. Microbial pathogenesis 24, 309-320, doi:10.1006/mpat.1997.0200; 1998). However, in Mtb-infected macrophages this Ca²⁺ elevation has been found to be reduced, thereby blocking phagosome-lysosome fusion and facilitating mycobacterial survival and long-term persistence (latency) (Majeed, M., Perskvist, N., Ernst, J. D., Orselius, K. & Stendahl, O. Roles of calcium and annexins in phagocytosis and elimination of an attenuated strain of Mycobacterium tuberculosis in human neutrophils. Microbial pathogenesis 24, 309-320, doi:10.1006/mpat.1997.0200; 1998). However, another study concluded that phagosome-lysosome fusion is a Ca²⁺-independent process (Zimmerli, S. et al. Phagosome-lysosome fusion is a calcium-independent event in macrophages. The Journal of cell biology 132, 49-61; 1996).

Cholesterol storage and a failure in the fusion of late endosomes/lysosomes (LE/Lys) also occurs in the rare inherited neurodegenerative lysosomal storage disease, Niemann-Pick type C (NPC) (Vanier, M. T. Niemann-Pick disease type C. Orphanet J Rare Dis 5, 16, doi:10.1186/1750-1172-5-16; 2010). NPC is caused by mutations in either of two genes, NPC1 (95% of clinical cases) or NPC2 (Vanier, M. T. Niemann-Pick disease type C. Orphanet J Rare Dis 5, 16, doi:10.1186/1750-1172-5-16; 2010). The proteins they encode function in a common cell biological pathway (the NPC pathway) with defects in either gene resulting in identical clinical phenotypes. NPC1 encodes NPC1, a multi-membrane-spanning protein that resides in of the limiting LE/Lys membrane (Davies, J. P., Chen, F. W. & Ioannou, Y. A. Transmembrane molecular pump activity of Niemann-Pick C1 protein. Science 290, 2295-2298, doi:10.1126/science.290.5500.2295 290/5500/2295 [pii]; 2000). In contrast, NPC2 is a soluble lysosomal cholesterol-binding protein (Naureckiene, S. et al. Identification of HE1 as the second gene of Niemann-Pick C disease. Science 290, 2298-2301; 2000). It has been proposed that NPC1 and NPC2 exchange cholesterol, although whether the pathway serves primarily to efflux cholesterol or is a cholesterol regulated/sensing pathway that effluxes other substrates remains unresolved (Lloyd-Evans, E. & Platt, F. M. Lipids on trial: the search for the offending metabolite in Niemann-Pick type C disease. Traffic 11, 419-428, doi:TRA1032 [pii] 10.1111/j.1600-0854.2010.01032.x; 2010). The sequence of events that culminates in NPC disease in cells following pharmacological inactivation of NPC1 is an increase in the level of sphingosine in the LE/Lys. This is rapidly followed by decreased Ca²⁺ storage and Ca²⁺ release from the LE/Lys. This leads to downstream endocytic trafficking defects, failure in LE/Lys fusion (Lloyd-Evans, E. et al. Niemann-Pick disease type C1 is a sphingosine storage disease that causes deregulation of lysosomal calcium. Nat Med 14, 1247-1255, doi:nm.1876 [pii] 10.1038/nm.1876; 2008; Morgan, A. J., Platt, F. M., Lloyd-Evans, E. & Galione, A. Molecular mechanisms of endolysosomal Ca²⁺ signalling in health and disease. Biochem J 439, 349-374, doi:BJ20110949 [pii] 10.1042/BJ20110949; 2011) and the subsequent storage of cholesterol and glycosphingolipids in a distended endo-lysosomal compartment. In addition to storage of multiple lipids, NPC cells also accumulate autophagic vacuoles due to a failure in their clearance (Pacheco, C. D., Kunkel, R. & Lieberman, A. P. Autophagy in Niemann-Pick C disease is dependent upon Beclin-1 and responsive to lipid trafficking defects. Hum Mol Genet 16, 1495-1503, doi:10.1093/hmg/ddm100; 2007; Settembre, C., Fraldi, A., Rubinsztein, D. C. & Ballabio, A. Lysosomal storage diseases as disorders of autophagy. Autophagy 4, 113-114, doi:5227 [pii]; 2008). Intriguingly, many of these NPC cellular phenotypes (Lloyd-Evans, E. & Platt, F. M. Lipids on trial: the search for the offending metabolite in Niemann-Pick type C disease. Traffic 11, 419-428, doi:TRA1032 [pii] 10.1111/j.1600-0854.2010.01032.x; 2010) are also observed in Mtb-infected macrophages, including endocytic transport abnormalities, defective autophagy, accumulation of free cholesterol, elevated levels of GSLs and the presence of lamellar storage bodies (Russell, D. G., Cardona, P. J., Kim, M. J., Allain, S. & Altare, F. Foamy macrophages and the progression of the human tuberculosis granuloma. Nat Immunol 10, 943-948, doi:10.1038/ni.1781; 2009).

### SUMMARY OF THE INVENTION

The observations outlined above prompted the inventors to investigate whether there is a mechanistic link between pathogenic mycobacterial infection and the NPC pathway. The inventors unexpectedly found that pathogenic mycobacteria, including Mycobacterium tuberculosis, secrete lipids that inhibit the NPC pathway, promoting the intra-cellular survival of the mycobacteria in host macrophages. This link between the rare lysosomal storage disorder NPC and Mtb infection has important implications for understanding host-pathogen interactions and for developing new therapies to combat TB, particularly in the current era of antibiotic resistance. The inventors found that treatment with a particular class of pharmacological agent that corrects defects in NPC1 mutant cells promotes mycobacterial clearance. In particular, SERCA antagonists, which inhibit Ca²⁺ uptake by the sarco-endoplasmic reticulum Ca²⁺-ATPase (SERCA), causing Ca²⁺ to leak from the endoplasmic reticulum and cytosolic [Ca²⁺ ] to increase, were found to significantly lower bacterial load. Other classes of agent known to be useful against NPC disease, however, did not promote mycobacterial clearance. In particular, miglustat, which is an inhibitor of glycosphingolipid (GSL) biosynthesis that is clinically approved for treating NPC disease, did not by itself enhance mycobacterial clearance over the time course tested. Also, β-cyclodextrin, which ameliorates NPC disease symptoms in animal models, possibly via stimulation of exocytosis, was found not to promote mycobacterial clearance. Furthermore, a structural analog of curcumin that does not function as SERCA antagonist was found not to promote clearance of the mycobacterium. These data support that the lipids secreted by the pathogenic mycobacteria inhibit the NPC pathway to achieve altered acidic store calcium homeostasis, that in turn blocks the ability of the lysosome to fuse and kill the mycobacterium, leading to persistence.

Despite the fact that the inhibitor of glycosphingolipid biosynthesis and cyclodextrin were found to be inactive, in that neither class of agent promoted mycobacterial clearance over the time course, it was unexpectedly found that inhibitors of glycosphingolipid biosynthesis show synergy when combined with a SERCA antagonist. Indeed, the combination of a SERCA antagonist (which elevates cytosolic calcium) and an inhibitor of glycosphingolipid biosynthesis showed significantly greater efficacy than the SERCA antagonist on its own and, of course, than the inhibitor of glycosphingolipid biosynthesis on its own which showed no activity. No such synergy was observed between β-cyclodextrin and SERCA antagonists. The invention therefore provides a surprisingly efficacious combination therapy for treating infections by pathogenic mycobacteria, and in particular infections by Mtb, which employs a SERCA antagonist in combination with an inhibitor of glycosphingolipid biosynthesis.

Accordingly, the present invention provides a combination comprising a SERCA antagonist and an inhibitor of glycosphingolipid biosynthesis, which combination is for use in the treatment of an infection by a pathogenic mycobacterium.

The active compounds in the combination, i.e. the SERCA antagonist and the inhibitor of glycosphingolipid biosynthesis, may be administered together in the same pharmaceutical composition or in different compositions intended for separate, simultaneous, concomitant or sequential administration by the same or a different route.

In one embodiment the SERCA antagonist and the inhibitor of glycosphingolipid biosynthesis are both present in the same pharmaceutical composition. Thus, the combination of the invention may comprise a pharmaceutical composition comprising the SERCA antagonist and the inhibitor of glycosphingolipid biosynthesis. The pharmaceutical composition may further comprise a pharmaceutically acceptable carrier or diluents.

Accordingly, the invention also provides a pharmaceutical composition comprising a SERCA antagonist, an inhibitor of glycosphingolipid biosynthesis, and a pharmaceutically acceptable carrier or diluent, which composition is for use in the treatment of an infection by a pathogenic mycobacterium.

The invention also provides a SERCA antagonist, which SERCA antagonist is for use in the treatment of an infection by a pathogenic mycobacterium by simultaneous, concurrent, separate or sequential co-administration with an inhibitor of glycosphingolipid biosynthesis.

The invention also provides an inhibitor of glycosphingolipid biosynthesis, which inhibitor of glycosphingolipid biosynthesis is for use in the treatment of an infection by a pathogenic mycobacterium by simultaneous, concurrent, separate or sequential co-administration with a SERCA antagonist.

The invention also provides a method of treatment of an infection by a pathogenic mycobacterium, which method comprises administering to a human or animal patient in need of such treatment an effective amount of a SERCA antagonist and an effective amount of an inhibitor of glycosphingolipid biosynthesis.

The active compounds employed in the method of the invention, i.e. the SERCA antagonist and the inhibitor of glycosphingolipid biosynthesis, may be administered together in the same pharmaceutical composition. Alternatively, they may be administered in different compositions, and the different compositions may be administered separately, simultaneously, concomitantly or sequentially, and by the same route or by a different route.

The invention also provides a kit of parts comprising a SERCA antagonist together with instructions for simultaneous, concurrent, separate or sequential use in combination with an inhibitor of glycosphingolipid biosynthesis, for the treatment of a human or animal patient suffering from or susceptible to an infection by a pathogenic mycobacterium.

The invention also provides a kit of parts comprising an inhibitor of glycosphingolipid biosynthesis together with instructions for simultaneous, concurrent, separate or sequential use in combination with a SERCA antagonist, for the treatment of a human or animal patient suffering from or susceptible to an infection by a pathogenic mycobacterium.

The invention also provides a kit of parts comprising an inhibitor of glycosphingolipid biosynthesis, a SERCA antagonist, and instructions for their simultaneous, concurrent, separate or sequential use for the treatment of a human or animal patient suffering from or susceptible to an infection by a pathogenic mycobacterium.

Preferably said patient is human.

### BRIEF DESCRIPTION OF THE FIGURES

FIG. 1A is a histogram showing that BCG infected macrophages exhibited elevated sphingosine levels relative to uninfected cells 48 hours post-infection.

FIG. 1B presents graphs which show that a 24-hour infection with BCG was associated with a reduction in GPN-induced Ca²⁺ release, whereas infection with non-pathogenic Mycobacterium smegmatis (M. smegmatis) was not.

FIG. 1C presents a histogram and micrographs showing the results of direct measurement of endo-lysosomal Ca²⁺ content with a luminal Ca²⁺-dye (low-affinity Rhod-dextran), and confirming the reduced levels of lysosomal Ca²⁺ in BCG-infected RAW cells.

FIG. 1D is a histogram demonstrating the elevated GSL NPC phenotype: total GSL levels were significantly elevated by 48 hours.

FIG. 2A is a histogram showing that, accumulation of lactosylceramide (LacCer), which occurs in NPC cells and in tissues of Npc1^{-/-} mice was not detected at 24 and 48 hours post-infection (BCG-infected RAW 264.7 cells) but was significantly elevated 7 days post-infection.

FIG. 2B contains micrographs showing cholesterol accumulation in punctate structures in BCG infected RAW 264.7 cells, but not in cells infected with non-pathogenic M. Smegmatis.

FIG. 2C is a histogram of the results of biochemical quantitation of cholesterol in control and BCG-infected cells, showing the higher cholesterol level in BCG-infected cells.

FIG. 3A contains micrographs showing that the following hallmarks of NPC were induced by BCG infection but not by M. smegmatis: the storage and mislocalisation (altered trafficking) of GM1 ganglioside (FIG. 3Ai) and sphingomyelin respectively (FIG. 3Aii).

FIG. 3B presents a histogram showing statistically significant elevation of GSLs in Mtb-infected cells together with representative HPLC traces.

FIG. 3C is a histogram showing increased levels of sphingosine in monocyte-derived macrophages from healthy donors infected with BCG compared to controls.

FIG. 3D is a histogram showing reduced LE/Lys Ca²⁺ levels (indirectly assayed by GPN-evoked Ca²⁺ release) in monocyte-derived macrophages from healthy donors infected with BCG compared to controls.

FIG. 4A is a histogram showing elevated GSL levels in monocyte-derived macrophages from healthy donors infected with BCG compared to controls.

FIG. 4B presents micrographs showing the detection of cholesterol storage in the LE/Lys in BCG-infected human macrophages (and in non-infected neighbouring cells) (FIG. 4Bi), accompanied by mistrafficking of GM1 ganglioside (FIG. 4Bii). Significant expansion of the lysosomal compartment was also detected, another hallmark of NPC (FIG. 4Biii). None of these changes occurred when human macrophages were infected with non-pathogenic M. smegmatis (FIG. 4Bi-iii).

FIG. 5A presents micrographs showing that chloroform: methanol-extracted BCG lipids induced accumulation/re-distribution of cholesterol (FIG. 5Ai), mistrafficking of GM1 ganglioside (FIG. 5Aii) and accumulation/re-distribution of sphingomyelin (FIG. 5Aiii) in uninfected RAW 264.7 macrophages, comparable to the NPC phenotypes induced by live mycobacteria.

FIG. 5B presents micrographs showing that a heat-treated preparation of the chloroform: methanol-extracted BCG lipids had the same ability to affect GM1 ganglioside distribution as the non-heat-treated fraction, suggesting that the NPC phenotype-inducing agent was a lipid and not proteinaceous.

FIG. 5C presents micrographs showing that Mtb-derived mycolic acids (the major cell wall-derived class of lipids in Mtb) induced storage of cholesterol and sphingomyelin in RAW 246.7 macrophages (FIG. 5Ci and ii).

FIG. 6A presents micrographs showing that Mtb-derived mycolic acids induced cholesterol storage and GM1 mistrafficking in primary human macrophages.

FIG. 6B is a histogram showing that, of Mtb derived trehalose dimycolate (TDM), trehalose monomycolate (TMM) and glucose monomycolate (GMM), TDM gave the greatest increase in Lysotracker fluorescence, indicative of NPC-like lysosomal expansion/storage, whereas GMM and TMM caused only modest lysosomal expansion that did not reach statistical significance, and GMM from non-pathogenic M. smegmatis had minimal effect.

FIG. 7 presents histograms, which show that, in addition to affecting lysosomal morphology, TDM from M bovis also recapitulated NPC phenotypes in terms of reduced LE/Lys Ca²⁺ levels.

FIG. 8 presents micrographs showing that TDM from M. bovis also recapitulated NPC phenotypes in terms of cholesterol storage (FIG. 8i) and GM1 mistrafficking (FIG. 8ii).

FIG. 9 is a histogram showing that TDM from M. bovis also recapitulated NPC phenotypes in terms of GSL accumulation.

FIG. 10A is a histogram showing that bone marrow-derived macrophages with 50% of wild-type NPC1 protein levels (heterozygous NPC1 cells) were more susceptible to inhibition by TDM than wild-type cells.

FIG. 10B presents micrographs demonstrating that CHO cells overexpressing NPC1 were more resistant to induction of NPC cellular phenotypes than wild-type cells when exposed to TDM. The extent of NPC1 overexpression was proportional to the resistance to phenotype induction; cells over-expressing NPC1 by 15-fold were more resistant than those over-expressing NPC1 5-fold.

FIG. 11 shows the expression levels of NPC1 and NPC2 proteins in RAW264.7 cells infected with BCG: expression of NPC1 protein was up regulated in infected cells, but there were no changes in NPC2 levels.

FIG. 12 presents (i) micrographs and (ii) a histogram showing that RAW 246.7 cells in which an NPC phenotype was pharmacologically induced by incubation with U18666A, prior to infection, had impaired ability to clear non-pathogenic M. smegmatis relative to untreated cells.

FIG. 13 is a histogram showing impaired clearance of M. smegmatis in Npc1^{-/-} bone marrow-derived mouse macrophages and U18666A-treated wild-type mouse macrophages.

FIG. 14 is a histogram showing impaired clearance of M. smegmatis in primary human macrophages treated with U18666A.

FIG. 15 presents a fluorescence trace and a histogram showing the frequency and extent of mycobacterial infection of RAW264.7 cells treated with mCherry-expressing BCG for 48 hours, then treated with or without 30 μM high-purity curcumin for a further 24 hours, by quantifying the fluorescence of mCherry-expressing M. bovis BCG using a FACS assay.

FIG. 16 presents two histograms (i and ii) showing: (i, ii) that a significantly lower level of infection (i.e. enhanced clearance) was observed in cells incubated with curcumin relative to untreated cells; (i) that neither miglustat alone nor β-cyclodextrin alone promoted mycobacterial clearance over the time course; (i) that miglustat in combination with curcumin showed synergy; (ii) that no synergy was seen when curcumin and cyclodextrin were used in combination.

FIG. 17 is a histogram showing that curcumin significantly reduced mycobacterial burden in infected primary human macrophages.

FIG. 18 presents a graph and a histogram showing that, whilst high-purity curcumin (SERCA antagonist) promoted BCG clearance, the inactive curcumin analogue FLLL31 (not a SERCA antagonist) had no effect.

FIG. 19 is a graph showing that bacterial clearance by curcumin was abrogated by loading cells with the Ca²⁺ chelator, BAPTA, to suppress a cytosolic Ca²⁺ increase.

### DETAILED DESCRIPTION OF THE INVENTION

### Substituent Definitions

The following definitions apply to the SERCA antagonists and the inhibitors of glycosphingolipid biosynthesis defined herein:

A C₁₋₂₀ alkyl group is an unsubstituted or substituted, straight or branched chain saturated hydrocarbon radical. Typically it is C₁₋₁₀ alkyl, for example methyl, ethyl, propyl, butyl, pentyl, hexyl, heptyl, octyl, nonyl or decyl, or C₁₋₆ alkyl, for example methyl, ethyl, propyl, butyl, pentyl or hexyl, or C₁₋₄ alkyl, for example methyl, ethyl, i-propyl, n-propyl, t-butyl, s-butyl or n-butyl. When an alkyl group is substituted it typically bears one or more substituents selected from substituted or unsubstituted C₁₋₂₀ alkyl, substituted or unsubstituted aryl (as defined herein), cyano, amino, C₁₋₁₀ alkylamino, di(C₁₋₁₀)alkylamino, arylamino, diarylamino, arylalkylamino, amido, acylamido, hydroxy, oxo, halo, carboxy, ester, acyl, acyloxy, C₁₋₂₀ alkoxy, aryloxy, haloalkyl, sulfonic acid, sulfhydryl (i.e. thiol, —SH), C₁₋₁₀ alkylthio, arylthio, sulfonyl, phosphoric acid, phosphate ester, phosphonic acid and phosphonate ester. Examples of substituted alkyl groups include haloalkyl, hydroxyalkyl, aminoalkyl, alkoxyalkyl and alkaryl groups. The term alkaryl, as used herein, pertains to a C₁₋₂₀ alkyl group in which at least one hydrogen atom has been replaced with an aryl group. Examples of such groups include, but are not limited to, benzyl (phenylmethyl, PhCH₂—), benzhydryl (Ph₂CH—), trityl (triphenylmethyl, Ph₃C—), phenethyl (phenylethyl, Ph-CH₂CH₂—), styryl (Ph-CH═CH—), cinnamyl (Ph-CH═CH—CH₂—). Typically a substituted C₁₋₂₀ alkyl group carries 1, 2 or 3 substituents, for instance 1 or 2.

A C₃₋₂₅ cycloalkyl group is an unsubstituted or substituted alkyl group which is also a cyclyl group; that is, a monovalent moiety obtained by removing a hydrogen atom from an alicyclic ring atom of a carbocyclic ring of a carbocyclic compound, which moiety has from 3 to 25 carbon atoms (unless otherwise specified), including from 3 to 25 ring atoms. Thus, the term “cycloalkyl” includes the sub-classes cycloalkyenyl and cycloalkynyl. Examples of groups of C₃₋₂₅ cycloalkyl groups include C₃₋₂₀ cycloalkyl, C₃₋₁₅ cycloalkyl, C₃₋₁₀ cycloalkyl, C₃₋₇ cycloalkyl. When a C₃₋₂₅ cycloalkyl group is substituted it typically bears one or more substituents selected from C₁₋₆ alkyl which is unsubstituted, aryl (as defined herein), cyano, amino, C₁₋₁₀ alkylamino, di(C₁₋₁₀)alkylamino, arylamino, diarylamino, arylalkylamino, amido, acylamido, hydroxy, oxo, halo, carboxy, ester, acyl, acyloxy, C₁₋₂₀ alkoxy, aryloxy, haloalkyl, sulfonic acid, sulfhydryl (i.e. thiol, —SH), C₁₋₁₀ alkylthio, arylthio, phosphoric acid, phosphate ester, phosphonic acid and phosphonate ester and sulfonyl. Typically a substituted C₃₋₂₅ cycloalkyl group carries 1, 2 or 3 substituents, for instance 1 or 2.

Examples of C₃₋₂₅ cycloalkyl groups include, but are not limited to, those derived from saturated monocyclic hydrocarbon compounds, which C₃₋₂₅ cycloalkyl groups are unsubstituted or substituted as defined above:

cyclopropane (C₃), cyclobutane (C₄), cyclopentane (C₅), cyclohexane (C₆), cycloheptane (C₇), methylcyclopropane (C₄), dimethylcyclopropane (C₈), methylcyclobutane (C₈), dimethylcyclobutane (C₆), methylcyclopentane (C₆), dimethylcyclopentane (C₇), methylcyclohexane (C₇), dimethylcyclohexane (C₈), menthane (C₁₀);

unsaturated monocyclic hydrocarbon compounds:

cyclopropene (C₃), cyclobutene (C₄), cyclopentene (C₈), cyclohexene (C₆), methylcyclopropene (C₄), dimethylcyclopropene (C₈), methylcyclobutene (C₈), dimethylcyclobutene (C₆), methylcyclopentene (C₆), dimethylcyclopentene (C₇), methylcyclohexene (C₇), dimethylcyclohexene (C₈);

saturated polycyclic hydrocarbon compounds:

thujane (C₁₀), carane (C₁₀), pinane (C₁₀), bornane (C₁₀), norcarane (C₇), norpinane (C₇), norbornane (C₇), adamantane (C₁₀), decalin (decahydronaphthalene) (C₁₀);

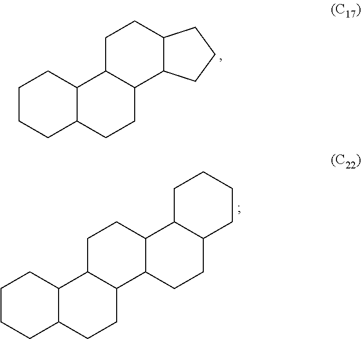

unsaturated polycyclic hydrocarbon compounds: camphene (C₁₀), limonene (C₁₀), pinene (C₁₀),

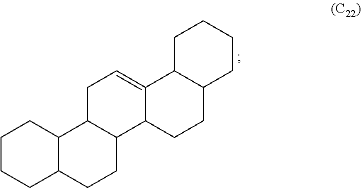 [SMILES: C1=C2C3CCCCC3CCC2C2CCC3CCCCC3C2C1]

polycyclic hydrocarbon compounds having an aromatic ring:

indene (C₉), indane (e.g., 2,3-dihydro-1H-indene) (C₉), tetraline (1,2,3,4-tetrahydronaphthalene) (C₁₀), acenaphthene (C₁₂), fluorene (C₁₃), phenalene (C₁₃), acephenanthrene (C₁₅), aceanthrene (C₁₆), cholanthrene (C₂₀).

A C₃₋₂₀ heterocyclyl group is an unsubstituted or substituted monovalent moiety obtained by removing a hydrogen atom from a ring atom of a heterocyclic compound, which moiety has from 3 to 20 ring atoms (unless otherwise specified), of which from 1 to 10 are ring heteroatoms. Preferably, each ring has from 3 to 7 ring atoms, of which from 1 to 4 are ring heteroatoms. When a C₃₋₂₀ heterocyclyl group is substituted it typically bears one or more substituents selected from C₁₋₆ alkyl which is unsubstituted, aryl (as defined herein), cyano, amino, C₁₋₁₀ alkylamino, di(C₁₋₁₀)alkylamino, arylamino, diarylamino, arylalkylamino, amido, acylamido, hydroxy, oxo, halo, carboxy, ester, acyl, acyloxy, C₁₋₂₀ alkoxy, aryloxy, haloalkyl, sulfonic acid, sulfhydryl (i.e. thiol, —SH), C₁₋₁₀ alkylthio, arylthio, phosphoric acid, phosphate ester, phosphonic acid and phosphonate ester and sulfonyl. Typically a substituted C₃₋₂₀ heterocyclyl group carries 1, 2 or 3 substituents, for instance 1 or 2.

Examples of groups of heterocyclyl groups include C₃₋₂₀ heterocyclyl, C₅₋₂₀heterocyclyl, C₃₋₁₅heterocyclyl, C₅₋₁₅heterocyclyl, C₃₋₁₂heterocyclyl, C₅₋₁₂heterocyclyl, C₃₋₁₀heterocyclyl, C₃₋₁₀heterocyclyl, C₃₋₇heterocyclyl, C₃₋₇heterocyclyl, and C₅₋₆heterocyclyl.

Examples of (non-aromatic) monocyclic C₃₋₂₀ heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₈), 2H-pyrrole or 3H-pyrrole (isopyrrole, isoazole) (C₈), piperidine (C₆), dihydropyridine (C₆), tetrahydropyridine (C₆), azepine (C₇);

O₁: oxirane (C₃), oxetane (C₄), oxolane (tetrahydrofuran) (C₈), oxole (dihydrofuran) (C₈), oxane (tetrahydropyran) (C₆), dihydropyran (C₆), pyran (C₆), oxepin (C₇);

S₁: thiirane (C₃), thietane (C₄), thiolane (tetrahydrothiophene) (C₈), thiane (tetrahydrothiopyran) (C₆), thiepane (C₇);

O₂: dioxolane (C₈), dioxane (C₆), and dioxepane (C₇);

O₃: trioxane (C₆);

N₂: imidazolidine (C₅), pyrazolidine (diazolidine) (C₈), imidazoline (C₈), pyrazoline (dihydropyrazole) (C₈), piperazine (C₆);

N₁O₁: tetrahydrooxazole (C₈), dihydrooxazole (C₈), tetrahydroisoxazole (C₈), dihydroisoxazole (C₈), morpholine (C₆), tetrahydrooxazine (C₆), dihydrooxazine (C₆), oxazine (C₆);

N₁S₁: thiazoline (C₈), thiazolidine (C₈), thiomorpholine (C₆);

N₂O₁: oxadiazine (C₆);

O₁S₁: oxathiole (C₈) and oxathiane (thioxane) (C₆); and,

N₁O₁S₁: oxathiazine (C₆).

Examples of substituted (non-aromatic) monocyclic heterocyclyl groups include those derived from saccharides, in cyclic form, for example, furanoses (C₅), such as arabinofuranose, lyxofuranose, ribofuranose, and xylofuranse, and pyranoses (C₆), such as allopyranose, altropyranose, glucopyranose, mannopyranose, gulopyranose, idopyranose, galactopyranose, and talopyranose. C₃₋₂₀ heterocyclyl includes groups derived from heterocyclic compounds of the following structure:

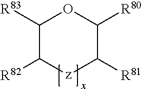

wherein x is 0 or 1; z is CHR⁸⁴; and R⁸⁰, R⁸¹, R⁸², R⁸³ and R⁸⁴, which are the same or different, are independently selected from H, C₁₋₆ alkyl, OH, acyloxy, SH, C₁₋₆ alkoxy, aryloxy, amino, C₁₋₁₀ alkylamino, di(C₁₋₁₀)alkylamino, amido, acylamido and a group derived from a second group of the following structure:

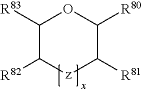

in which second group x is 0 or 1; z is CHR⁸⁴; and R⁸⁰, R⁸¹, R⁸², R⁸³ and R⁸⁴, which are the same or different, are independently selected from H, C₁₋₆ alkyl, OH, acyloxy, SH, C₁₋₆ alkoxy, aryloxy, amino, C₁₋₁₀ alkylamino, di(C₁₋₁₀)alkylamino, amido and acylamido. The term “group derived from” in this case means that the group is a monovalent moiety obtained by removing the R⁸⁰, R⁸¹, R⁸², R⁸³ or R⁸⁴ atom from a carbon atom of the above compounds. Thus, C₃₋₂₀ heterocyclyl includes groups of the following structure:

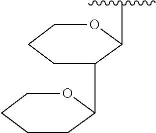 [SMILES: *C1OCCCC1C1CCCCO1]

wherein each of the ring carbon atoms is independently unsubstituted or substituted with C₁₋₆ alkyl, OH, acyloxy, SH, C₁₋₆ alkoxy, aryloxy, amino, C₁₋₁₀ alkylamino, di(C₁₋₁₀)alkylamino, amido and acylamido.

C₃₋₂₀ heterocyclyl also includes groups in which two heterocyclic rings are linked by an oxygen atom. Thus, C₃₋₂₀ heterocyclyl includes disaccharide groups, in which two monosaccharide heterocyclic rings are linked with an oxygen atom. Accordingly, C₃₋₂₀ heterocyclyl includes groups of the following formula (m):

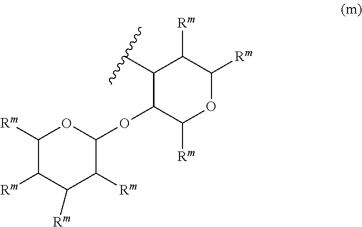

wherein each R^{m}, which is the same or different, is independently selected from C₁₋₆ alkyl, OH, acyloxy, SH, C₁₋₆ alkoxy, aryloxy, amino, C₁₋₁₀ alkylamino, di(C₁₋₁₀)alkylamino, amido and acylamido. Thus, the following disaccharide group is one example of a substituted C₃₋₂₀ heterocyclic group:

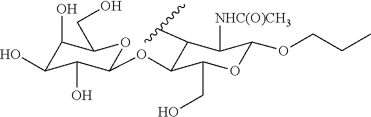

Examples of C₃₋₂₀ heterocyclyl groups which are also aryl groups are described below as heteroaryl groups.

An aryl group is a substituted or unsubstituted, monocyclic or bicyclic aromatic group which typically contains from 6 to 14 carbon atoms, preferably from 6 to 10 carbon atoms in the ring portion. Examples include phenyl, naphthyl, indenyl and indanyl groups. An aryl group is unsubstituted or substituted. When an aryl group as defined above is substituted it typically bears one or more substituents selected from C₁-C₆ alkyl which is unsubstituted (to form an aralkyl group), aryl which is unsubstituted, cyano, amino, C₁₋₁₀alkylamino, di(C₁₋₁₀)alkylamino, arylamino, diarylamino, arylalkylamino, amido, acylamido, hydroxy, halo, carboxy, ester, acyl, acyloxy, C₁₋₂₀ alkoxy, aryloxy, haloalkyl, sulfhydryl (i.e. thiol, —SH), C₁₋₁₀alkylthio, arylthio, sulfonic acid, phosphoric acid, phosphate ester, phosphonic acid and phosphonate ester and sulfonyl. Typically it carries 0, 1, 2 or 3 substituents. A substituted aryl group may be substituted in two positions with a single C₁₋₆ alkylene group, or with a bidentate group represented by the formula —X—C₁₋₆ alkylene, or —X—C₁₋₆ alkylene-X—, wherein X is selected from O, S and NR, and wherein R is H, aryl or C₁₋₆ alkyl. Thus a substituted aryl group may be an aryl group fused with a cycloalkyl group or with a heterocyclyl group. The term aralkyl as used herein, pertains to an aryl group in which at least one hydrogen atom (e.g., 1, 2, 3) has been substituted with a C₁₋₆ alkyl group. Examples of such groups include, but are not limited to, tolyl (from toluene), xylyl (from xylene), mesityl (from mesitylene), and cumenyl (or cumyl, from cumene), and duryl (from durene). The ring atoms of an aryl group may include one or more heteroatoms (as in a heteroaryl group). Such an aryl group (a heteroaryl group) is a substituted or unsubstituted mono- or bicyclic heteroaromatic group which typically contains from 6 to 10 atoms in the ring portion including one or more heteroatoms. It is generally a 5- or 6-membered ring, containing at least one heteroatom selected from O, S, N, P, Se and Si. It may contain, for example, 1, 2 or 3 heteroatoms. Examples of heteroaryl groups include pyridyl, pyrazinyl, pyrimidinyl, pyridazinyl, furanyl, thienyl, pyrazolidinyl, pyrrolyl, oxazolyl, oxadiazolyl, isoxazolyl, thiadiazolyl, thiazolyl, isothiazolyl, imidazolyl, pyrazolyl, quinolyl and isoquinolyl. A heteroaryl group may be unsubstituted or substituted, for instance, as specified above for aryl. Typically it carries 0, 1, 2 or 3 substituents.

A C₁₋₂₀ alkylene group is an unsubstituted or substituted bidentate moiety obtained by removing two hydrogen atoms, either both from the same carbon atom, or one from each of two different carbon atoms, of a hydrocarbon compound having from 1 to 20 carbon atoms (unless otherwise specified), which may be aliphatic or alicyclic, and which may be saturated, partially unsaturated, or fully unsaturated. Thus, the term “alkylene” includes the sub-classes alkenylene, alkynylene, cycloalkylene, etc., discussed below.

Typically it is C₁₋₁₀ alkylene, for instance C₁₋₆ alkylene. Typically it is C₁₋₄ alkylene, for example methylene, ethylene, i-propylene, n-propylene, t-butylene, s-butylene or n-butylene. It may also be pentylene, hexylene, heptylene, octylene and the various branched chain isomers thereof. An alkylene group may be unsubstituted or substituted, for instance, as specified above for alkyl. Typically a substituted alkylene group carries 1, 2 or 3 substituents, for instance 1 or 2.

In this context, the prefixes (e.g., C₁₋₄, C₁₋₇, C₁₋₂₀, C₂₋₇, C₃₋₇, etc.) denote the number of carbon atoms, or range of number of carbon atoms. For example, the term “C₁₋₄alkylene,” as used herein, pertains to an alkylene group having from 1 to 4 carbon atoms. Examples of groups of alkylene groups include C₁₋₄ alkylene (“lower alkylene”), C₁₋₇ alkylene, C₁₋₁₀ alkylene and C₁₋₂₀ alkylene.

Examples of linear saturated C₁₋₇ alkylene groups include, but are not limited to, —(CH₂)ₙ— where n is an integer from 1 to 7, for example, —CH₂— (methylene), —CH₂CH₂-(ethylene), —CH₂CH₂CH₂— (propylene), and —CH₂CH₂CH₂CH₂— (butylene).

Examples of branched saturated C₁₋₇ alkylene groups include, but are not limited to, —CH(CH₃)—, —CH(CH₃)CH₂—, —CH(CH₃)CH₂CH₂—, —CH(CH₃)CH₂CH₂CH₂—, —CH₂CH(CH₃)CH₂—, —CH₂CH(CH₃)CH₂CH₂—, —CH(CH₂CH₃)—, —CH(CH₂CH₃)CH₂—, and —CH₂CH(CH₂CH₃)CH₂—.

Examples of linear partially unsaturated C₁₋₇ alkylene groups include, but is not limited to, —CH═CH— (vinylene), —CH═CH—CH₂—, —CH₂—CH═CH₂—, —CH═CH—CH₂—CH₂—, —CH═CH—CH₂—CH₂—CH₂—, —CH═CH—CH═CH—, —CH═CH—CH═CH—CH₂—, —CH═CH—CH═CH—CH₂—CH₂—, —CH═CH—CH₂—CH═CH—, and —CH═CH—CH₂—CH₂—CH═CH—.

Examples of branched partially unsaturated C₁₋₇ alkylene groups include, but is not limited to, —C(CH₃)═CH—, —C(CH₃)═CH—CH₂—, and —CH═CH—CH(CH₃)—.

Examples of alicyclic saturated C₁₋₇ alkylene groups include, but are not limited to, cyclopentylene (e.g., cyclopent-1,3-ylene), and cyclohexylene (e.g., cyclohex-1,4-ylene).

Examples of alicyclic partially unsaturated C₁₋₇ alkylene groups include, but are not limited to, cyclopentenylene (e.g., 4-cyclopenten-1,3-ylene), cyclohexenylene (e.g., 2-cyclohexen-1,4-ylene; 3-cyclohexen-1,2-ylene; 2,5-cyclohexadien-1,4-ylene).

C₁₋₂₀ alkylene and C₁₋₂₀ alkyl groups as defined herein are either uninterrupted or interrupted by one or more heteroatoms or heterogroups, such as S, O or N(R″) wherein R″ is H, C₁₋₆ alkyl or aryl (typically phenyl), or by one or more arylene (typically phenylene) groups. The phrase “optionally interrupted” as used herein thus refers to a C₁₋₂₀ alkyl group or an alkylene group, as defined above, which is uninterrupted or which is interrupted between adjacent carbon atoms by a heteroatom such as oxygen or sulfur, by a heterogroup such as N(R″) wherein R″ is H, aryl or C₁-C₆ alkyl, or by an arylene group. For instance, a C₁₋₂₀ alkyl group such as n-butyl may be interrupted by the heterogroup N(R″) as follows: —CH₂N(R″)CH₂CH₂CH₃, —CH₂CH₂N(R″)CH₂CH₃, or —CH₂CH₂CH₂N(R″)CH₃. Similarly, an alkylene group such as n-butylene may be interrupted by the heterogroup N(R″) as follows: —CH₂N(R″)CH₂CH₂CH₂—, —CH₂CH₂N(R″)CH₂CH₂—, or —CH₂CH₂CH₂N(R″)CH₂—. Typically an interrupted group, for instance an interrupted C₁₋₂₀ alkylene or C₁₋₂₀ alkyl group, is interrupted by 1, 2 or 3 heteroatoms or heterogroups or by 1, 2 or 3 arylene (typically phenylene) groups. More typically, an interrupted group, for instance an interrupted C₁₋₂₀ alkylene or C₁₋₂₀ alkyl group, is interrupted by 1 or 2 heteroatoms or heterogroups or by 1 or 2 arylene (typically phenylene) groups. For instance, a C₁₋₂₀ alkyl group such as n-butyl may be interrupted by 2 heterogroups N(R″) as follows: —CH₂N(R″)CH₂N(R″)CH₂CH₃.

An arylene group is an unsubstituted or substituted bidentate moiety obtained by removing two hydrogen atoms, one from each of two different aromatic ring atoms of an aromatic compound, which moiety has from 5 to 14 ring atoms (unless otherwise specified). Typically, each ring has from 5 to 7 or from 5 to 6 ring atoms. An arylene group may be unsubstituted or substituted, for instance, as specified above for aryl.

In this context, the prefixes (e.g., C₅₋₂₀, C₆₋₂₀, C₅₋₁₄, C₅₋₇, C₅₋₆, etc.) denote the number of ring atoms, or range of number of ring atoms, whether carbon atoms or heteroatoms. For example, the term “C₅₋₆ arylene,” as used herein, pertains to an arylene group having 5 or 6 ring atoms. Examples of groups of arylene groups include C₅₋₂₀ arylene, C₆₋₂₀ arylene, C₅₋₁₄ arylene, C₆₋₁₄ arylene, C₆₋₁₀ arylene, C₅₋₁₂ arylene, C₅₋₁₀ arylene, C₅₋₇ arylene, C₅₋₆ arylene, C₅ arylene, and C₆ arylene.

The ring atoms may be all carbon atoms, as in “carboarylene groups” (e.g., C₆₋₂₀ carboarylene, C₆₋₁₄ carboarylene or C₆₋₁₀ carboarylene).

Examples of C₆₋₂₀ arylene groups which do not have ring heteroatoms (i.e., C₆₋₂₀ carboarylene groups) include, but are not limited to, those derived from the compounds discussed above in regard to aryl groups, e.g. phenylene, and also include those derived from aryl groups which are bonded together, e.g. phenylene-phenylene (diphenylene) and phenylene-phenylene-phenylene (triphenylene).

Alternatively, the ring atoms may include one or more heteroatoms, as in “heteroarylene groups” (e.g., C₅₋₁₀ heteroarylene).

Examples of C₅₋₁₀ heteroarylene groups include, but are not limited to, those derived from the compounds discussed above in regard to heteroaryl groups.

As used herein the term oxo represents a group of formula: ═O As used herein the term acyl represents a group of formula: —C(═O)R, wherein R is an acyl substituent, for example, a substituted or unsubstituted C₁₋₂₀ alkyl group, a substituted or unsubstituted C₃₋₂₀ heterocyclyl group, or a substituted or unsubstituted aryl group. Examples of acyl groups include, but are not limited to, —C(═O)CH₃ (acetyl), —C(═O)CH₂CH₃ (propionyl), —C(═O)C(CH₃)₃ (t-butyryl), and —C(═O)Ph (benzoyl, phenone).

As used herein the term acyloxy (or reverse ester) represents a group of formula: —OC(═O)R, wherein R is an acyloxy substituent, for example, substituted or unsubstituted C₁₋₂₀ alkyl group, a substituted or unsubstituted C₃₋₂₀heterocyclyl group, or a substituted or unsubstituted aryl group, typically a C₁₋₆ alkyl group. Examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

As used herein the term ester (or carboxylate, carboxylic acid ester or oxycarbonyl) represents a group of formula: —C(═O)OR, wherein R is an ester substituent, for example, a substituted or unsubstituted C₁₋₂₀ alkyl group, a substituted or unsubstituted C₃₋₂₀ heterocyclyl group, or a substituted or unsubstituted aryl group (typically a phenyl group). Examples of ester groups include, but are not limited to, —C(═O)OCH₃, —C(═O)OCH₂CH₃, —C(═O)OC(CH₃)₃, and —C(═O)OPh.

As used herein the term phosphonic acid represents a group of the formula: —P(═O)(OH)₂. As would be understood by the skilled person, a phosphonic acid group can exist in protonated and deprotonated forms (i.e. —P(═O)(OH)₂, —P(═O)(O⁻)₂ and —P(═O)(OH)(O⁻)) all of which are within the scope of the term “phosphonic acid”.

As used herein the term phosphonic acid salt represents a group which is a salt of a phosphonic acid group. For example a phosphonic acid salt may be a group of the formula —P(═O)(OH)(O⁻X⁺) wherein X is a monovalent cation. X⁺ may be an alkali metal cation. X⁺ may be Na⁺ or K⁺, for example.

As used herein the term phosphonate ester represents a group of one of the formulae:

—P(═O)(OR)₂ and —P(═O)(OR)O⁻ wherein each R is independently a phosphonate ester substituent, for example, —H, substituted or unsubstituted C₁₋₂₀ alkyl, substituted or unsubstituted C₃₋₂₀ heterocyclyl, C₃₋₂₀ heterocyclyl substituted with a further C₃₋₂₀ heterocyclyl, substituted or unsubstituted C₁₋₂₀ alkylene-C₃₋₂₀ heterocyclyl, substituted or unsubstituted C₃₋₂₅ cycloalkyl, substituted or unsubstituted C₁₋₂₀ alkylene-C₃₋₂₅ cycloalkyl, aryl, substituted or unsubstituted C₁₋₂₀ alkylene-aryl. Examples of phosphonate ester groups include, but are not limited to, —P(═O)(OCH₃)₂, —P(═O)(OCH₂CH₃)₂, —P(═O)(O-t-Bu)₂, and —P(═O)(OPh)₂,

As used herein the term phosphoric acid represents a group of the formula: —OP(═O)(OH)₂.

As used herein the term phosphate ester represents a group of one of the formulae: —OP(═O)(OR)₂ and —OP(═O)(OR)O⁻ wherein each R is independently a phosphate ester substituent, for example, —H, substituted or unsubstituted C₁₋₂₀ alkyl, substituted or unsubstituted C₃₋₂₀ heterocyclyl, C₃₋₂₀ heterocyclyl substituted with a further C₃₋₂₀ heterocyclyl, substituted or unsubstituted C₁₋₂₀ alkylene-C₃₋₂₀ heterocyclyl, substituted or unsubstituted C₃₋₂₅ cycloalkyl, substituted or unsubstituted C₁₋₂₀ alkylene-C₃₋₂₅ cycloalkyl, aryl, substituted or unsubstituted C₁₋₂₀ alkylene-aryl. Examples of phosphate ester groups include, but are not limited to, —OP(═O)(OCH₃)₂, —OP(═O)(OCH₂CH₃)₂, —OP(═O)(O-t-Bu)₂, and —OP(═O)(OPh)₂.

As used herein the term amino represents a group of formula —NH₂. The term C₁-C₁₀ alkylamino represents a group of formula —NHR′ wherein R′ is a C₁₋₁₀ alkyl group, preferably a C₁₋₆ alkyl group, as defined previously. The term di(C₁₋₁₀)alkylamino represents a group of formula —NR′R″ wherein R′ and R″ are the same or different and represent C₁₋₁₀ alkyl groups, preferably C₁₋₆ alkyl groups, as defined previously. The term arylamino represents a group of formula —NHR′ wherein R′ is an aryl group, preferably a phenyl group, as defined previously. The term diarylamino represents a group of formula —NR′R″ wherein R′ and R″ are the same or different and represent aryl groups, preferably phenyl groups, as defined previously. The term arylalkylamino represents a group of formula —NR′R″ wherein R′ is a C₁₋₁₀ alkyl group, preferably a C₁₋₆ alkyl group, and R″ is an aryl group, preferably a phenyl group.

As used herein the term amido represents a group of formula: —C(═O)NR′R″, wherein R′ and R″ are independently amino substituents, as defined for di(C₁₋₁₀)alkylamino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R′ and R″, together with the nitrogen atom to which they are attached, form a heterocyclic structure as in, for example, piperidinocarbonyl, morpholinocarbonyl, thiomorpholinocarbonyl, and piperazinocarbonyl.

As used herein the term acylamido represents a group of formula: —NR¹C(═O)R², wherein R¹ is an amide substituent, for example, hydrogen, a C₁₋₂₀alkyl group, a C₃₋₂₀ heterocyclyl group, an aryl group, preferably hydrogen or a C₁₋₂₀ alkyl group, and R² is an acyl substituent, for example, a C₁₋₂₀ alkyl group, a C₃₋₂₀ heterocyclyl group, or an aryl group, preferably hydrogen or a C₁₋₂₀ alkyl group. Examples of acylamide groups include, but are not limited to, —NHC(═O)CH₃, —NHC(═O)CH₂CH₃, —NHC(═O)Ph, —NHC(═O)C₁₅H₃₁ and —NHC(═O)C₉H₁₉. Thus, a substituted C₁₋₂₀ alkyl group may comprise an acylamido substituent defined by the formula —NHC(═O)—C₁₋₂₀ alkyl, such as —NHC(═O)C₁₅H₃₁ or —NHC(═O)C₉H₁₉. R¹ and R² may together form a cyclic structure, as in, for example, succinimidyl, maleimidyl, and phthalimidyl:

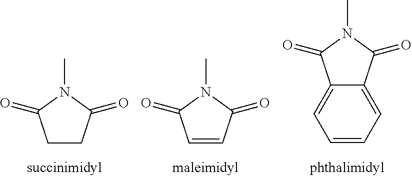

A C₁₋₁₀ alkylthio group is a said C₁₋₁₀ alkyl group, preferably a C₁₋₆ alkyl group, attached to a thio group. An arylthio group is an aryl group, preferably a phenyl group, attached to a thio group.

A C₁₋₂₀ alkoxy group is a said substituted or unsubstituted C₁₋₂₀ alkyl group attached to an oxygen atom. A C₁₋₆ alkoxy group is a said substituted or unsubstituted C₁₋₆ alkyl group attached to an oxygen atom. A C₁₋₄ alkoxy group is a substituted or unsubstituted C₁₋₄ alkyl group attached to an oxygen atom. Said C₁₋₂₀, C₁₋₆ and C₁₋₄ alkyl groups are optionally interrupted as defined herein. Examples of C₁₋₄ alkoxy groups include, —OMe (methoxy), —OEt (ethoxy), —O(nPr) (n-propoxy), —O(iPr) (isopropoxy), —O(nBu) (n-butoxy), —O(sBu) (sec-butoxy), —O(iBu) (isobutoxy), and —O(tBu) (tert-butoxy). Further examples of C₁₋₂₀ alkoxy groups are —O(Adamantyl), —O—CH₂-Adamantyl and —O—CH₂—CH₂-Adamantyl. An aryloxy group is a substituted or unsubstituted aryl group, as defined herein, attached to an oxygen atom. An example of an aryloxy group is —OPh (phenoxy).

Unless otherwise specified, included in the above are the well known ionic, salt, solvate, and protected forms of these substituents. For example, a reference to carboxylic acid or carboxyl group (—COOH) also includes the anionic (carboxylate) form (—COO—), a salt or solvate thereof, as well as conventional protected forms. Similarly, a reference to an amino group includes the protonated form (—N⁺HR¹R²), a salt or solvate of the amino group, for example, a hydrochloride salt, as well as conventional protected forms of an amino group. Similarly, a reference to a hydroxyl group also includes the anionic form (—O⁻), a salt or solvate thereof, as well as conventional protected forms.

Certain compounds may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and 1-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers,” as used herein, are structural (or constitutional) isomers (i.e., isomers which differ in the connections between atoms rather than merely by the position of atoms in space). For example, a reference to a methoxy group,—OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto, enol, and enolate forms, as in, for example, the following tautomeric pairs: keto/enol (illustrated below), imine/enamine, amide/imino alcohol, amidine/amidine, nitroso/oxime, thioketone/enethiol, N-nitroso/hyroxyazo, and nitro/aci-nitro.

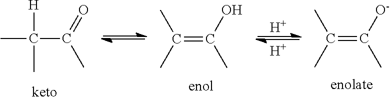

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof.

Methods for the preparation (e.g., asymmetric synthesis) and separation (e.g., fractional crystallisation and chromatographic means) of such isomeric forms are either known in the art or are readily obtained by adapting known methods, in a known manner.

Unless otherwise specified, a reference to a particular compound also includes ionic, salt, solvate, protected forms and prodrugs thereof.

Examples of pharmaceutically acceptable salts of the compounds for use in accordance with the present invention include salts with inorganic acids such as hydrochloric acid, hydrobromic acid, hydroiodic acid, sulphuric acid, nitric acid and phosphoric acid; and organic acids such as methanesulfonic acid, benzenesulphonic acid, formic acid, acetic acid, trifluoroacetic acid, propionic acid, butyric acid, isobutyric acid, oxalic acid, malonic acid, succinic acid, fumaric acid, maleic acid, lactic acid, malic acid, tartaric acid, citric acid, ethanesulfonic acid, aspartic acid, benzoic acid and glutamic acid. Typically the salt is a hydrochloride, an acetate, a propionate, a benzoate, a butyrate or an isobutyrate. Examples of pharmaceutically acceptable salts are discussed in Berge et al., 1977, “Pharmaceutically Acceptable Salts,” J. Pharm. Sci., Vol. 66, pp. 1-19.

A prodrug of a SERCA antagonist or an inhibitor of glycosphingolipid biosynthesis, is a compound which, when metabolised (e.g., in vivo), yields the desired active compound. Typically, the prodrug is inactive, or less active than the active compound, but may provide advantageous handling, administration, or metabolic properties.

For example, some prodrugs are O-acylated (acyloxy) derivatives of the active compound, i.e. physiologically acceptable metabolically labile acylated derivatives. During metabolism, the one or more —O-acyl (acyloxy) groups (—O—C(═O)R^{p}) are cleaved to yield the active drug. R^{p} may be a C₁₋₁₀alkyl group, an aryl group or a C₃₋₂₀ cycloalkyl group. Typically, R^{p} is a C₁₋₁₀ alkyl group including, but not limited to, methyl, ethyl, propyl, butyl, pentyl, hexyl, heptyl, octyl, nonyl and decyl. Such derivatives may be formed by acylation, for example, of any of the hydroxyl groups (—OH) in the parent compound, with, where appropriate, prior protection of any other reactive groups present in the parent compound, followed by deprotection if required. Thus, the free hydroxyl groups on an iminosugar inhibitor of glycosphingolipid biosynthesis (for instance DNJ, DGJ, or an N-alkylated derivative of DNJ or DGJ such as NB-DNJ or NB-DGJ) may be acylated with up to four, typically exactly four, O-acyl groups. The O-acyl groups are enzymatically removed in vivo to provide the non-O-acylated (i.e. hydroxyl-containing) active inhibitor of glycosphingolipid biosynthesis.

Some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

The SERCA antagonist and the inhibitor of glycosphingolipid biosynthesis can be used in the free form or the salt form. Either or both agents may also be used in prodrug form. The prodrug can itself be used in the free form or the salt form.

### SERCA Antagonists

SERCA antagonists are known compounds. J. Med. Chem. 2005, 48, 7005-7011 describes thapsigargin and various analogs thereof as potent SERCA antagonists, and compares the respective activities of the compounds. Curcumin is also a SERCA antagonist; Eur. J. Biochem, 268, 6318-6327 (2001) details the SERCA inhibitory activity of curcumin. Cyclopiazonic acid is also a known SERCA antagonist.

SERCA antagonists may be readily identified, without undue experimentation, using known procedures. J. Med. Chem. 2005, 48, 7005-7011 describes how SERCA antagonists may be designed (for instance, using the structure of a known SERCA antagonist as a starting point) and then tested for SERCA inhibitory activity. A standard assay for the measurement of SERCA inhibitory activity is described in J. Med. Chem. 2005, 48, p 7010. Eur. J. Biochem, 268, 6318-6327 (2001) also describes how Ca²⁺ ATPase activity can be determined, in order to identify SERCA antagonists, using a phosphate liberation assay described in Longland et al., Cell Calcium 24, 27-34 (1998).

The SERCA antagonist employed in the present invention may for instance be selected from curcumin, cyclopiazonic acid and a compound of formula (A):

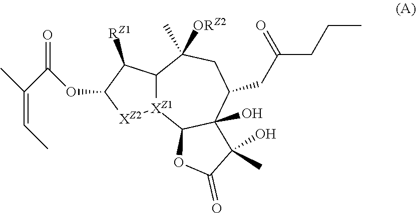

wherein:

R^{Z1} is selected from hydrogen, hydroxyl, carboxyl, amino, thiol, halo, substituted or unsubstituted C₁₋₁₀alkyl, substituted or unsubstituted C₁₋₁₀alkoxy, substituted or unsubstituted aryloxy, acyl, ester, acyloxy, C₁₋₁₀ alkylamino, di(C₁₋₁₀)alkylamino, amido, acylamido, —O—C₃₋₂₅ cycloalkyl and —O—C₃₋₂₀ heterocyclyl, wherein said C₁₋₁₀alkyl is optionally interrupted by N(R′), O, S or arylene;

R^{Z2} is selected from hydrogen, substituted or unsubstituted C₁₋₁₀alkyl and acyl, wherein said C₁₋₁₀alkyl is optionally interrupted by N(R′), O, S or arylene; and

either X^{Z1} is CH and X^{Z2} is CHR′ wherein X^{Z1} and X^{Z2} are bonded by a C—C single bond, or X^{Z1} is C and X² is CR′ wherein X^{Z1} and X^{Z2} are bonded by a C═C double bond; and R′ is H, C₁₋₆ alkyl or aryl;

and pharmaceutically acceptable salts thereof.

Typically, R^{Z1} is selected from H and ester. More typically, R^{Z1} is selected from H and —OC(O)(CH₂)₆CH₃.

Typically, R^{Z2} is selected from C₁₋₁₀ alkyl and acyl. More typically, R^{Z2} is selected from ethyl and acetyl (i.e. —C(O)CH₃).

Typically, X^{Z1} is CH and X² is C(H)Me. Alternatively, X^{Z1} is C and X^{Z2} is C(H)Me.

In one embodiment, R^{Z1} is —OC(O)(CH₂)₆CH₃, R^{Z2} is —C(O)CH₃, X^{Z1} is C and X^{Z2} is C(H)Me (i.e. the compound of formula A is thapsigargin).

In another embodiment, R^{Z1} is H, R^{Z2} is —C(O)CH₃, X^{Z1} is C and X^{Z2} is C(H)Me (i.e. the compound of formula A is nortrilobolide).

In another embodiment, R^{Z1} is H, R^{Z2} is ethyl, X^{Z1} is CH and X^{Z2} is C(H)Me.

In another embodiment, R^{Z1} is H, R^{Z2} is —C(O)CH₃, X^{Z1} is CH and X^{Z2} is C(H)Me.

The SERCA antagonist employed in the present invention may for instance be selected from curcumin, thapsigargin, nortrilobolide and cyclopiazonic acid, and pharmaceutically acceptable salts thereof.

More typically, the SERCA antagonist is selected from thapsigargin, curcumin and cyclopiazonic acid, and pharmaceutically acceptable salts thereof.

The chemical structure of curcumin is as follows:

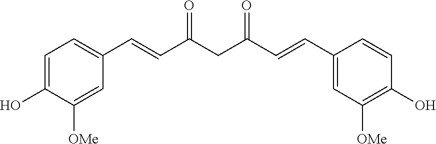 [SMILES: COc1cc(/C=C/C(=O)CC(=O)/C=C/c2ccc(O)c(OC)c2)ccc1O]

The structure of thapsigargin is as follows:

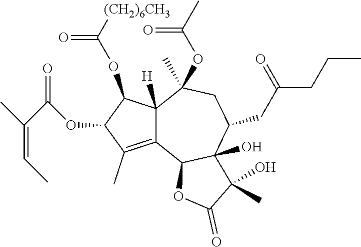

In a particularly preferred embodiment of the present invention, the SERCA antagonist is curcumin or a pharmaceutically acceptable salt thereof.

In the most preferred embodiment of the invention, the SERCA antagonist is curcumin.

### Inhibitors of Glycosphingolipid Biosynthesis

The term “inhibitor of glycosphingolipid biosynthesis”, as used herein, means a compound that is capable of inhibiting the synthesis or expression of a glycosphingolipid (GSL).

Typically, the GSL is a ganglioside. Alternatively, the GSL is a neutral GSL.

Inhibitors of glycosphingolipid biosynthesis are either known or readily identifiable, without undue experimentation, using known procedures.

GSLs are synthesized from ceramide by the sequential addition of monosaccharides mediated by Golgi-resident glycosyltransferases. The two main classes of GSL are the neutral GSLs (lacto and globo series) and the gangliosides. Gangliosides contain sialic acid (neuraminic acid) and are consequently negatively charged. The majority of GSLs are glucose derivatives of ceramide. However, galactose based GSLs are also present and are particularly abundant in the CNS. Such galactose-based GSLs include the sulfatides.

A class of compounds which can affect the metabolism of glycosphingolipids, is that of formula (I) defined below. Some of these compounds (notably, NB-DNJ, miglustat) have found use in the treatment of congenital disorders of glycolipid storage (such as type I Gaucher disease, reviewed in Aerts J M et al. J. Inherit Metab. Dis. (2006) 29(2-3): 449-453) and as potential anti-microbial agents (for example to modulate the toxicity of cholera toxin to ganglioside-type glycolipids, reviewed in Svensson M et al. Mol Microbiol. (2003) 47: 453-461).

Defects in GSL catabolism result in a build up of GSLs. Such diseases are termed GSL storage disorders. Small-molecule inhibitors such as the alkyl-iminosugars have been developed to inhibit the biosynthesis of glucosylceramide, the first step in the biosynthesis of GSLs. Such compounds are thus inhibitors of glycosphingolipid biosynthesis which may be employed in the present invention. Glucosylceramide is synthesised by the action of glucosylceramide synthase (also known as UDP-glucose: N-acylsphingosine glucosyltransferase), which catalyses the transfer of glucose to ceramide. The inhibition of glucosylceramide synthase can be achieved in vivo by small-molecule inhibitors (Reviewed in Asano N. Glycobiology (2003) 13:93-104). Inhibition can be achieved by small-molecule mimics of the substrate, transition state or product of glucosylceramide synthase. Such inhibitors include: (1) mimics of the carbohydrate moiety (“sugar mimics”), and (2) mimics of the ceramide or sphingosine moiety (“lipid mimics”).

The sugar mimics (1) have received considerable attention and include iminosugars such as nojirimycin, N-butyldeoxynojirimycin (NB-DNJ) and N-butyldeoxygalactonojirimycin (NB-DGJ) (see U.S. Pat. No. 5,472,969; U.S. Pat. No. 5,656,641; U.S. Pat. No. 6,465,488; U.S. Pat. No. 6,610,703; U.S. Pat. No. 6,291,657; U.S. Pat. No. 5,580,884; Platt F, J. Biol. Chem. (1994) 269:8362-8365; Platt F M et al. Phil. Trans. R. Soc. Lond. B (2003) 358:947-954; and Butters T D et al. Glycobiology (2005) 15:43-52). The modification of the iminosugar core with an alkyl chain such as a butyl group (as in NB-DNJ) or a nonyl group (as in NN-DNJ) are important for the clinical applications. Further sugar derivatives include N-(5-adamantane-1-yl-methoxypentyl)-DNJ (AMP-DNJ) (Overkleeft et al. J. Biol. Chem. (1998) 41:26522-26527), α-homogalactonojirimycin (HGJ) (Martin et al. 1995 Tetrahedron Letters 36:10101-10116), α-homoallonojirimycin (HAJ) (Asano et al 1997 J. Nat. Prod. 60:98-101, Martin et al 1999 Bioorg. Med. Chem. Lett 9:3171-3174) and β-1-C-butyl-DGJ (CB-DGJ) (Ikeda et al 2000 Carbohydrate Res. 323:73-80). NB-DNJ results in measurable decrease in GSL levels within a day of treatment with the effect on GSL levels stabilizing after 10 days of treatment in mice (Platt FM J. Biol. Chem. (1997) Aug. 1; 272(31):19365-72.). Critically, both NB-DNJ and NB-DGJ penetrate the CNS without significant effects on behaviour or CNS development, and treatment of adult mice with NB-DNJ or NB-DGJ has been shown not to cause neurological side effects (U. Andersson et al., Neurobiology of Disease, 16 (2004) 506-515). NB-DGJ resulted in a marked reduction in total ganglioside and GM1 content in cerebrum-brainstem (Kasperzyk et al. J. Lipid Res. (2005) 46:744-751).

Lipid mimics (2) have been developed to inhibit glycosphingolipid biosynthesis (Abe A. et al. J. Biochem Tokyo (1992) 111:191-196. Reviewed in Asano N. Glycobiology (2003) 13:93-104). Ceramide-based inhibitors include D,L-threo-1-phenyl-2-decanoylamino-3-morpholino-1-propanol (PDMP) and D,L-threo-1-phenyl-2-hexadecanoylamino-3-morpholino-1-propanol (PPMP). Numerous derivatives have subsequently been developed such as: D-threo-1-phenyl-2-palmitoilamino-3-pyrrolidino-1-propanol (P4), 4′-hydroxy-P4 (pOH-P4), 3′,4′-ethylenedioxy-P4 (EtDO-P4; Genz-78132, Genzyme), N-[(1R,2R)-1-(2,3-Dihydro-1,4-benzodioxin-6-yl)-1-hydroxy-3-(1-pyrrolidinyl)-2-propanyl]octanamide (eliglustat; Genz-99067) and salts thereof, including the eliglustat salt eliglustat tartrate (Genz-112638; Cerdelga) which has the IUPAC name: N-[(1R,2R)-1-(2,3-dihydro-1,4-benzodioxin-6-yl)-1-hydroxy-3-pyrrolidin-1-ylpropan-2-yl]octanamide; (2R,3R)-2,3-dihydroxybutanedioic acid. L-DMDP (Yu C Y et al. Chem Commun (Camb). 2004 Sep. 7; (17):1936-7) has also been developed. Small-molecule inhibitors of galactosyltransferases have also been developed and are described in Chung S J, Bioorg Med Chem Lett. 1998 Dec. 1; 8(23):3359-64.

Accordingly, in one embodiment of the invention the inhibitor of glycosphingolipid biosynthesis is an inhibitor of a glycosyltransferase. Typically, the inhibitor mimics the substrate, transition state or product of the glycosyltransferase. In particular, the inhibitor may be a compound that mimics the carbohydrate moiety of the substrate, transition state or product of the glycosyltransferase. Alternatively, the inhibitor is a compound that mimics the lipid moiety of the substrate, transition state or product of the glycosyltransferase. Typically, the glycosyltransferase is a glucosyltransferase. The glucosyltransferase is, for instance, glucosylceramide synthase. Alternatively, the glycosyltransferase may be a galactosyltransferase. The galactosyltransferase may be, for instance, β1-4 galactosyltransferase. Alternatively, the glycosyltransferase may be a ceramide galactosyltransferase. The ceramide galactosyltransferase may be, for instance, UDP-galactose:ceramide galactosyltransferase (also known as galactosylceramide synthase).

In principle, all glycosyltransferases can be inhibited by substrate mimics (Chung S J, Bioorg Med Chem Lett. 1998 Dec. 1; 8(23):3359-64). Such substrate mimics can be employed for use in the present invention as inhibitors of glycosphingolipid biosynthesis.

The skilled person can readily identify inhibitors of glycosphingolipid biosynthesis without undue experimentation, using known procedures. For instance, inhibitors of glycosphingolipid biosynthesis can be identified by incubating and or growing cells in culture in the presence of the putative inhibitor together with an assay for the effect of glycosphingolipid biosynthesis. Such assays include the analysis of fluorescently-labelled glycosphingolipid carbohydrate headgroups by HPLC, thin-layer chromatography (TLC) of glycosphingolipids and analysis of glycosphingolipids using mass spectrometry (Neville DC, Anal. Biochem. 2004 Aug. 15; 331(2):275-82; Mellor HR Biochem. J. 2004 Aug. 1; 381(Pt 3):861-6; Hayashi Y. et al., Anal. Biochem. 2005 Oct. 15; 345(2):181-6; Sandhoff, R. et al., J. Biol. Chem., vol. 277, no. 23, 20386-20398, 2002; Sandhoff, R. et al., J. Biol. Chem., vol. 280, no. 29, 27310-27318, 2005; Platt, F. M. et al., J. Biol. Chem., vol. 269, issue 11, 8362-8365, 1994; Platt, F. M. et al., J. Biol. Chem., vol. 269, issue 43, 27108-27114, 1994).

Neville D C et al. (Anal. Biochem. 2004 Aug. 15; 331(2):275-82) have developed an optimised assay method in which fluorescently labelled glycosphingolipid-derived oligosaccharides are analysed. Thus, inhibitors of glycosphingolipid biosynthesis for use in accordance with the present invention can be identified by incubating or growing cells in culture, in the presence of the putative inhibitor, and applying the assay described in Neville et al. The assay described in Neville et al. enables GSL levels to be measured by HPLC analysis of GSL-derived oligosaccharides following ceramide glycanase digestion of the GSLs and anthranilic acid labelling of the released oligosaccharides. In the assay, glyocosphingolipids (GSLs) are extracted from the sample and purified by column chromatography. The extracted GSLs are then digested with ceramide glycanase. The extracted GSLs are first dried and redissolved, with mixing in 10 μl incubation buffer (50 μM sodium acetate, pH 5.0, containing 1 mg/ml sodium cholate or sodium taurodeoxycholate). To this is added, with gentle mixing, 0.05 U ceramide glycanase in a further 10 μl incubation buffer (giving a final concentration of 2.5 U/ml). One unit (U) is defined as the amount of enzyme that will hydrolyze 1.0 nmol of ganglioside, GM1, per minute at 37° C. Incubations are performed at 37° C. for 18 hours. The ceramide-glycanase-released oligosaccharides are then labelled with anthranilic acid and purified essentially as described in Anumula and Dhume, Glycobiology 8 (1998) 685-694 with the modifications described in Neville D C et al. Anal. Biochem. 2004 Aug. 15; 331(2):275-82. The purified 2-AA-labelled oligosaccharides are then separated by normal phase HPLC, as described in Neville D C et al., and glucose unit values are determined following comparison with a 2-AA-labelled glucose oligomer ladder (derived from a partial hydrolysate of dextran) external standard. Inhibitors of glycosphingolipid biosynthesis are identified by measuring the decrease in GSL levels observed in the presence of the inhibitor. A similar assay method is described in Mellor HR Biochem. J. 2004 Aug. 1; 381(Pt 3):861-6. That document describes the synthesis of a series of DNJ analogues to study their inhibitory activity in cultured HL60 cells. When the cells are treated for 16 hours at non-cytotoxic concentrations of DNJ analogue, a 40-50% decrease in GSL levels can be observed by HPLC analysis of GSL-derived oligosaccharides following ceramide glycanase digestion of GSL and 2-aminobenzamide labelling of the released oligosaccharides.

Hayashi Y. et al., Anal. Biochem. 2005 Oct. 15; 345(2):181-6 reports an HPLC-based method that uses fluorescent acceptors and nonradioisotope UDP-sugar donors to provide a fast, sensitive and reproducible assay to determine glucosylceramide synthase (GlcT) and lactosylceramide synthase (GalT) activities. Thus, inhibitors of glycosphingolipid biosynthesis for use in accordance with the present invention can be identified by incubating and or growing cells in culture in the presence of the putative inhibitor, and applying the assay method described in Hayashi et al. The HPLC-based assay procedures described in Hayashi et al. involve mixing a fluorescent acceptor substrate, either 50 pmol of C6-NBD-Cer or C6-NBD-GlcCer, and 6.5 nmol of lecithin in 100 μmol of ethanol, and then evaporating the solvent. Next 10 μl of water is added and the mixture is sonicated to form liposomes. For the GlcT assay, 50 μl of reaction mixture contains 500 μM UDP-Glc, 1 mM EDTA, 10 μl C6-NBD-Cer liposome and 20 μl of an appropriate amount of enzyme in lysis buffer 1. For the GalT assay, 50 μl of mixture contains 100 μM UDP-Gal, 5 mM MgCl₂, 5 mM MnCl₂, 10 μl C6-NBD-GlcCer liposome, and 20 μl of an appropriate amount of enzyme in lysis buffer 2. The assays are carried out at 37° C. for 1 hour. The reaction is stopped by adding 200 μl of chloroform/methanol (2:1, v/v). After a few seconds of vortexing, 5 μl of 500 μM KCl is added and then centrifuged. After the organic phase has dried up, lipids are dissolved in 200 μl of isopropyl alcohol/n-hexane/H₂O (55:44:1) and then transferred to a glass vial in an autosampler. A 100 μl aliquot sample is then loaded onto a normal-phase column and eluted with isopropyl alcohol/n-hexane/H₂O (55:44:1) for the GlcT assay or isopropyl alcohol/n-hexane/H₂O/phosphoric acid (110:84:5.9:0.1) for the GalT assay at a flow rate of 2.0 ml/min. Fluorescence can be determined using a fluorescent detector set to excitation and emission wavelengths of 470 and 530 nm, respectively. Fluorescent peaks are identified by comparing their retention times with those of standards.

Further assays include fluorescent-activated cells sorting (FACS) with glycosphingolipid-binding proteins such as anti-glycosphingolipid antibodies or lectins (see for example Rouquette-Jazdanian et al., The Journal of Immunology, 2005, 175: 5637-5648 and Chefalo, P et al., Biochemistry 2006, Mar. 21; 45(11): 3733-9).

Sandhoff et al. (J. Biol. Chem., vol. 277, no. 23, 20386-20398, 2002 and J. Biol. Chem., vol. 280, no. 29, 27310-27318, 2005) describe assay methods in which glycosphingolipids are analysed by mass spectrometry or by TLC. Inhibitors of glycosphingolipid biosynthesis for use in accordance with the present invention can be identified by incubating and or growing cells in culture in the presence of the putative inhibitor, and applying the TLC assay method or mass spectrometry assay method described by Sandhoff et al. Further details of these methods are given below.

In the methods described by Sandhoff et al. (J. Biol. Chem., vol. 277, no. 23, 20386-20398, 2002 and J. Biol. Chem., vol. 280, no. 29, 27310-27318, 2005) the glycosphingolipid profiles in mice were measured by nano-electrospray ionization tandem mass spectrometry (nano-ESI-MS/MS). Glycosphingolipids were first extracted from murine tissue for mass spectrometric analysis. The samples prepared included both the extracted GSLs and synthesised GSL MS standards. Nano-ESI-MS/MS analyses were performed with a triple quadropole instrument equipped with a nano-electrospray source operating at an estimated flow rate of 20-50 nl/min. Usually 10 μL of sample, dissolved in methanol or methanolic ammonium acetate (5 mM), was filled into a gold-sputtered capillary, which was positioned at a distance of 1-3 mm in front of the cone. The source temperature was set to 30° C. and the spray was started by applying 800-1200 V to the capillary. For each spectrum 20-50 scans of 15-30 s duration were averaged. The resulting Nano-ESI-MS/MS data could then be evaluated for quantification of the GSLs as follows: Quantitative spectra were measured with an average mass resolution of 1200 (ion mass/full width half maximum). Peak height values of the first mono-isotopic peak of each compound were taken for evaluation. A linear trend was calculated from the peak intensities of the corresponding internal standard lipids. The obtained calibration curve represented the intensity of the internal standard amount at a given m/z value. The quantities of the individual species of a GSL were calculated using a corrected intensity ratio (sample GSL/internal standard trend), knowing the amount of the internal standard added. The amount of the GSL was then calculated from the sum of the individual molecular species.

Sandhoff et al. (J. Biol. Chem., vol. 277, no. 23, 20386-20398, 2002 and J. Biol. Chem., vol. 280, no. 29, 27310-27318, 2005) also describe a procedure for analysing GSLs using TLC. Glycosphingolipids were extracted from murine tissue for analysis by TLC. Neutral and acidic GSL fractions were each taken up in 100 μL chloroform/methanol/water (10:10:1). Aliquots were then spotted on TLC plates with a Linomat IV from CAMAG (Muttenz, CH). A pre-run was performed with chloroform/alcohol (1:1). The plates were then dried and the GSLs were separated with the running solvent chloroform, methanol, 0.2% aqueous CaCl₂ (60:35:8). GSL bands were detected with orcinol/sulphuric acid spray reagent at 110° C. for 10 to 20 mins and the GSLs were identified by comparison with GSL standards.

TLC assays for analysing glycosphingolipid biosynthesis are also described in Platt, F. M. et al., J. Biol. Chem., vol. 269, issue 11, 8362-8365 and 1994; Platt, F. M. et al., J. Biol. Chem., vol. 269, issue 43, 27108-27114, 1994.

The inhibitor of glycosphingolipid biosynthesis employed in the present invention may be an inhibitor of glycosphingolipid biosynthesis of the following formula (I):

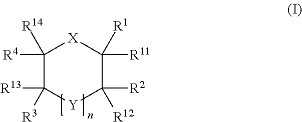

wherein:

X is O, S or NR⁵;

R⁵ is hydrogen, substituted or unsubstituted C₁₋₂₀ alkyl, substituted or unsubstituted C₁₋₂₀ alkylene-aryl, substituted or unsubstituted C₁₋₂₀ alkylene-C₃₋₂₀ heteroaryl, substituted or unsubstituted C₁₋₂₀ alkylene-C₃₋₂₅ cycloalkyl, substituted or unsubstituted C₁₋₂₀ alkylene-C₃₋₂₀ heterocyclyl, substituted or unsubstituted C₁₋₂₀ alkylene-O—C₃₋₂₀ heterocyclyl, substituted or unsubstituted aryl, substituted or unsubstituted C₃₋₂₀ heteroaryl, substituted or unsubstituted C₃₋₂₅ cycloalkyl or substituted or unsubstituted C₃₋₂₀ heterocyclyl, or R⁵ forms, together with R¹, R¹¹, R⁴ or R¹⁴, a substituted or unsubstituted C₁₋₆ alkylene group, wherein said C₁₋₂₀ alkyl and C₁₋₂₀ alkylene are optionally interrupted by N(R′), O, S or arylene wherein R′ is H, C₁₋₆ alkyl or aryl;

n is 0 or 1;

Y is O, S or CR⁶R¹⁶;

R¹, R¹¹, R⁴ and R¹⁴, which may be the same or different, are independently selected from hydrogen, hydroxyl, carboxyl, amino, thiol, substituted or unsubstituted C₁₋₂₀ alkyl, substituted or unsubstituted C₁₋₂₀ alkoxy, substituted or unsubstituted C₁₋₂₀ alkylene-C₃₋₂₅ cycloalkyl, substituted or unsubstituted aryloxy, acyl, ester, acyloxy, C₁₋₁₀ alkylamino, di(C₁₋₁₀)alkylamino, amido, acylamido, —O—C₃₋₂₅ cycloalkyl and —O—C₃₋₂₀ heterocyclyl, provided that one of R¹, R¹¹, R⁴ and R¹⁴ may form, together with R⁵, a substituted or unsubstituted C₁₋₆ alkylene group, wherein said C₁₋₂₀ alkyl and C₁₋₂₀ alkylene are optionally interrupted by N(R′), O, S or arylene; and

R², R¹², R³, R¹³, R⁶ and R¹⁶, which may be the same or different, are independently selected from hydrogen, hydroxyl, carboxyl, amino, thiol, substituted or unsubstituted C₁₋₂₀ alkyl, substituted or unsubstituted C₁₋₂₀ alkoxy, substituted or unsubstituted —O—C₁₋₂₀ alkylene-C₃₋₂₅ cycloalkyl, substituted or unsubstituted aryloxy, acyl, ester, acyloxy, C₁₋₁₀alkylamino, di(C₁₋₁₀)alkylamino, amido, acylamido, —O—C₃₋₂₅ cycloalkyl and —O—C₃₋₂₀ heterocyclyl, wherein said C₁₋₂₀ alkyl and C₁₋₂₀ alkylene are optionally interrupted by N(R′), O, S or arylene,

or a pharmaceutically acceptable salt thereof.

In the compounds of formula (I), typically either R¹ or R¹¹ (more typically R¹¹) is H. Typically, either R² or R¹² (more typically R¹²) is H. Typically, either R³ or R¹³ (more typically R¹³) is H. Typically, either R⁴ or R¹⁴ (more typically R¹⁴) is H. Typically, where Y is CR⁶R¹⁶, either R⁶ or R¹⁶ (more typically R¹⁶) is H.

Typically, R¹ or R¹¹ is selected from hydrogen, hydroxyl, carboxyl, substituted or unsubstituted C₁₋₂₀ alkyl, substituted C₁₋₂₀ alkoxy, substituted or unsubstituted aryloxy, —O—C₃₋₂₅ cycloalkyl and —O—C₃₋₂₀ heterocyclyl. More typically, R¹¹ is hydrogen and R¹ is selected from hydrogen, hydroxyl, carboxyl, substituted or unsubstituted C₁₋₂₀ alkyl, substituted C₁₋₂₀ alkoxy, substituted or unsubstituted aryloxy, —O—C₃₋₂₅ cycloalkyl and —O—C₃₋₂₀ heterocyclyl.

Typically, when R¹ or R¹¹ is C₁₋₂₀ alkoxy, said C₁₋₂₀ alkoxy group is substituted with an ester group or an aryl group, for instance with —C(O)OCH₃ or Ph.

Typically, when R¹ or R¹¹ is an aryloxy group, the aryl group bonded to the oxygen of said aryloxy is either substituted or unsubstituted phenyl, or substituted or unsubstituted naphthyl. Typically, the phenyl or naphthyl is either unsubstituted or monosubstituted with halo or methoxy.

When R¹ or R¹¹ is a substituted C₁₋₂₀ alkyl group, the substituent may be a hydroxyl, phosphate ester or phosphonate ester group. For instance, R¹ or R¹¹ may be CH₂OH or a group of the following formula (VII):

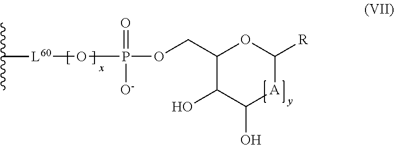

wherein L⁶⁰ is substituted or unsubstituted C₁₋₂₀ alkylene; x is 0 or 1; y is 0 or 1; A is CHR′″ and R is H, C₁₋₂₀ alkyl, C₃₋₂₀ heterocyclyl, C₃₋₂₅ cycloalkyl, aryl or C₁₋₂₀ alkoxy, wherein R′″ is hydroxyl, C₁₋₆ alkoxy, aryloxy or acyl. Typically R′″ is hydroxyl. Typically R is either —OCH₃ or a heterocyclic group of the following structure:

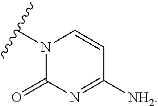 [SMILES: *n1ccc(N)nc1=O]

Typically, both R¹ and R¹¹ are groups of formula (VII) above. An example of a compound in which both R¹ and R¹¹ are groups of formula (VII) is cytidin-5′-yl sialylethylphosphonate.

Typically, when R¹ or R″ is unsubstituted C₁₋₂₀ alkyl, it is a methyl, ethyl, propyl, butyl, pentyl, hexyl, heptyl, octyl, nonyl or decyl group.

Typically, when R¹ or R″ is —O—C₃₋₂₅ cycloalkyl, the cycloalkyl group is a group derived from a compound of one of the following formulae, which compound may be substituted or unsubstituted:

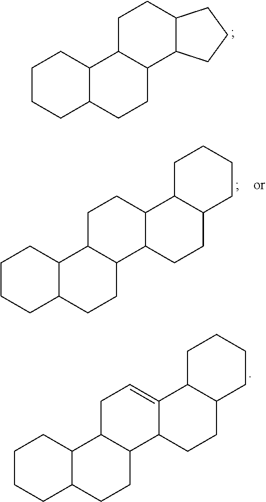

The term “group derived from a compound” in this case means that the group is a monovalent moiety obtained by removing a hydrogen atom from a carbon atom of the compound. An example of a compound in which R¹ or R¹¹ is —O—C₃₋₂₅ cycloalkyl is Soyasaponin I, in which R¹ or R¹¹ has the following structure:

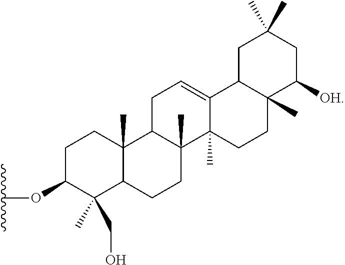

Typically, when R¹ or R¹¹ is —O—C₃₋₂₀ heterocyclyl, said heterocyclyl group is a group derived from a monosaccharide in cyclic form, for instance a group of the following structure:

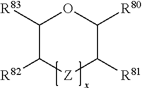

wherein x is 0 or 1; z is CHR⁸⁴; and R⁸⁰, R⁸¹, R⁸², R⁸³ and R⁸⁴, which are the same or different, are independently selected from H, C₁₋₆ alkyl, OH, acyloxy, SH, C₁₋₆ alkoxy, aryloxy, amino, C₁₋₁₀ alkylamino, di(C₁₋₁₀)alkylamino, amido and acylamido. The term “group derived from” in this case means that the group is a monovalent moiety obtained by removing the R⁸⁰, R⁸¹, R⁸², R⁸³ or R⁸⁴ atom from a carbon atom of the above compound.

More typically, when R¹ or R¹¹ is —O—C₃₋₂₀ heterocyclyl, said —O—C₃₋₂₀ heterocyclyl group is a group of any one of the following structures:

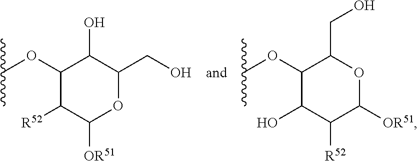

in which R⁵¹ is a substituted or unsubstituted C₁₋₁₀ alkyl group, typically methyl, or a substituted or unsubstituted aryl group, typically a phenyl or naphthyl group. The phenyl or naphthyl may be unsubstituted or substituted. When substituted, the phenyl or naphthyl is typically substituted with a halo group, for instance with a bromo group. R⁵² is typically hydroxyl, C₁₋₁₀ alkoxy, acyloxy, aryloxy or acylamido. Typically, R⁵² is —OH or —NHC(O)Me.

In the compounds of formula (I), typically either R² or R¹² is selected from hydrogen, hydroxyl, acyloxy, acylamido, C₁₋₂₀ alkoxy, C₁₋₂₀ alkyl and —O—C₃₋₂₀ heterocyclyl. More typically, R¹² is hydrogen and R² is selected from hydrogen, hydroxyl, acyloxy, C₁₋₂₀ alkoxy, C₁₋₂₀ alkyl and —O—C₃₋₂₀ heterocyclyl.

Typically, when R² or R¹² is acylamido, said acylamido is —NHC(O)CH₃.

Typically, when R² or R¹² is acyloxy, said acyloxy is selected from —OC(O)CH₃, —OC(O)CH₂CH₃, —OC(O)CH₂CH₂CH₃ and —OC(O)CH₂CH₂CH₂CH₃. More typically, when R² or R¹² is acyloxy, said acyloxy is —OC(O)CH₂CH₂CH₃.

Typically, when R² or R¹² is —O—C₃₋₂₀ heterocyclyl, said heterocyclyl group is a group derived from a monosaccharide in cyclic form, for instance a group of the following structure:

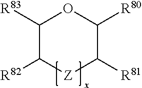

wherein x is 0 or 1; z is CHR⁸⁴; and R⁸⁰, R⁸¹, R⁸², R⁸³ and R⁸⁴, which are the same or different, are independently selected from H, C₁₋₆ alkyl, OH, SH, C₁₋₆ alkoxy, aryloxy, amino, C₁₋₁₀ alkylamino, di(C₁₋₁₀)alkylamino, amido, acylamido and a group derived from a second group of the following structure:

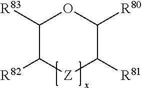

in which second group x is 0 or 1; z is CHR⁸⁴; and R⁸⁰, R⁸¹, R⁸², R⁸³ and R⁸⁴, which are the same or different, are independently selected from H, C₁₋₆ alkyl, OH, SH, C₁₋₆ alkoxy, aryloxy, amino, C₁₋₁₀ alkylamino, di(C₁₋₁₀)alkylamino, amido and acylamido. The term “group derived from” in this case means that the group is a monovalent moiety obtained by removing the R⁸⁰, R⁸¹, R⁸², R⁸³ or R⁸⁴ atom or group from a carbon atom of the compound. Thus when R² or R¹² is —O—C₃₋₂₀ heterocyclyl, said heterocyclyl group may be a group of the following structure:

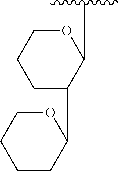 [SMILES: *C1OCCCC1C1CCCCO1]

wherein each of the ring carbon atoms is independently unsubstituted or substituted with C₁₋₆ alkyl, OH, SH, C₁₋₆ alkoxy, aryloxy, amino, C₁₋₁₀alkylamino, di(C₁₋₁₀)alkylamino, amido and acylamido. An example of a compound in which R² or R¹² is —O—C₃₋₂₀ heterocyclyl is Soyasaponin I, in which R² or R¹² is a group of the following structure:

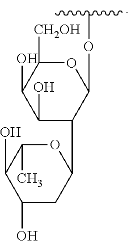

Typically, when R² or R¹² is C₁₋₂₀ alkoxy or C₁₋₂₀ alkyl, the group is methyl, ethyl, propyl, butyl, pentyl, hexyl, methoxy, ethoxy, propoxy, butoxy, pentoxy or hexoxy.

More typically, R² or R¹² is selected from H, OH, —OC(O)CH₂CH₂CH₃ and NHC(O)CH₃. In another embodiment, R² or R¹² is selected from H and OH.

In the compounds of formula (I), typically either R³ or R¹³ is selected from hydrogen, hydroxyl, acyloxy, acylamido, C₁₋₂₀ alkoxy and C₁₋₂₀ alkyl. More typically, R¹³ is hydrogen and R³ is selected from hydrogen, hydroxyl, acyloxy, C₁₋₂₀ alkoxy and C₁₋₂₀ alkyl.

Typically, when R³ or R¹³ is acylamido, said acylamido is —NHC(O)CH₃.

Typically, when R³ or R¹³ is acyloxy, said acyloxy is selected from —OC(O)CH₃, —OC(O)CH₂CH₃, —OC(O)CH₂CH₂CH₃ and —OC(O)CH₂CH₂CH₂CH₃. More typically, when R³ or R¹³ is acyloxy, said acyloxy is —OC(O)CH₂CH₂CH₃.

Typically, when R³ or R¹³ is C₁₋₂₀ alkoxy or C₁₋₂₀ alkyl, the group is methyl, ethyl, propyl, butyl, pentyl, hexyl, methoxy, ethoxy, propoxy, butoxy, pentoxy or hexoxy.

More typically, R³ or R¹³ is selected from H, OH and NHC(O)CH₃. In another embodiment, R³ or R¹³ is selected from H and OH.

In the compounds of formula (I), typically either R⁴ or R¹⁴ is hydrogen, hydroxyl, acyloxy, carboxyl, ester or C₁₋₂₀ alkyl which is substituted or unsubstituted, or R⁴ or R¹⁴ forms, together with R⁵, a substituted or unsubstituted C₁₋₆ alkylene group. More typically, R¹⁴ is hydrogen and R⁴ is hydrogen, hydroxyl, acyloxy, carboxyl, ester or C₁₋₂₀ alkyl which is substituted or unsubstituted, or R⁴ forms, together with R⁵, a substituted or unsubstituted C₁₋₆ alkylene group.

Typically, when R⁴ or R¹⁴ is acyloxy, said acyloxy is selected from —OC(O)CH₃, —OC(O)CH₂CH₃, —OC(O)CH₂CH₂CH₃ and —OC(O)CH₂CH₂CH₂CH₃.

Typically, when R⁴ or R¹⁴ is a C₁₋₂₀ alkyl, said C₁₋₂₀ alkyl is substituted with one, two, three or four groups selected from hydroxyl, acyloxy, thiol and —SC(O)R⁹⁵, wherein R⁹⁵ is C₁₋₆ alkyl. More typically, said C₁₋₂₀ alkyl is methyl, ethyl, propyl or butyl substituted with one, two, three or four groups respectively, which groups are selected from hydroxyl, acyloxy and thiol, more typically from hydroxyl and thiol.

Typically, when R⁴ or R¹⁴ forms, together with R⁵, a substituted or unsubstituted C₁₋₆ alkylene group, said alkylene group is substituted or unsubstituted propylene.

Typically, said propylene is unsubstituted or substituted with a C₁₋₄ alkyl group, for instance with a methyl group. Examples of compounds of formula (I) in which R⁴ or R¹⁴ forms, together with R⁵, a methyl-substituted propylene group are Castanospermine and MDL25874, whose structures are given below.

R⁴ or R¹⁴ is typically H, —CH₂OH, —CH₂SH, —CH(OH)CH(OH)CH₂OH or —COOH or R⁴ or R¹⁴ forms, together with R⁵, a propylene group substituted with a methyl group.

In the compounds of formula (I), typically n is 1, Y is CR⁶R¹⁶ and either R⁶ or R¹⁶ is selected from hydrogen, hydroxyl, acyloxy, amino, C₁₋₂₀ alkoxy and C₁₋₂₀ alkyl. More typically, n is 1, Y is CR⁶R¹⁶, R¹⁶ is hydrogen and R⁶ is selected from hydrogen, hydroxyl, acyloxy, amino, C₁₋₂₀ alkoxy and C₁₋₂₀ alkyl.

Typically, when R⁶ or R¹⁶ is acyloxy, said acyloxy is selected from —OC(O)CH₃, —OC(O)CH₂CH₃, —OC(O)CH₂CH₂CH₃ and —OC(O)CH₂CH₂CH₂CH₃.

Typically, when R⁶ or R¹⁶ is C₁₋₂₀ alkoxy or C₁₋₂₀ alkyl, the group is methyl, ethyl, propyl, butyl, pentyl, hexyl, methoxy, ethoxy, propoxy, butoxy, pentoxy or hexoxy.

Typically, R⁶ or R¹⁶ is selected from —OH and —NH₂.

Alternatively, n is 1 and Y is O or S. More typically, n is 1 and Y is O.

Alternatively, n is 0.

In the compounds of formula (I), typically R⁵ is hydrogen, substituted or unsubstituted C₁₋₂₀ alkyl, substituted or unsubstituted C₁₋₂₀ alkylene-aryl, substituted or unsubstituted C₁₋₂₀ alkylene-C₃₋₂₀ heteroaryl, substituted or unsubstituted C₁₋₂₀ alkylene-C₃₋₂₅ cycloalkyl, substituted or unsubstituted C₁₋₂₀ alkylene-C₃₋₂₀ heterocyclyl, substituted or unsubstituted C₁₋₂₀ alkylene-O—C₃₋₂₀ heterocyclyl or R⁵ forms, together with R¹, R¹¹, R⁴ or R¹⁴, a substituted or unsubstituted C₁₋₆ alkylene group, wherein said C₁₋₂₀ alkyl and C₁₋₂₀ alkylene are optionally interrupted by N(R′), O, S or arylene wherein R′ is H, C₁₋₆ alkyl or aryl.

Typically, when R⁵ is substituted or unsubstituted C₁₋₂₀ alkylene-C₃₋₂₅ cycloalkyl, the C₁₋₂₀ alkylene is interrupted once by O and the C₃₋₂₅ cycloalkyl is Adamantyl, and thus R⁵ is —(C₁₋₁₀ alkylene)-O—CH₂-Adamantyl. This includes, for instance, —(CH₂)₅—O—CH₂-Adamantyl.

Typically, when R⁵ is substituted or unsubstituted C₁₋₂₀ alkylene-O—C₃₋₂₀ heterocyclyl, said C₁₋₂₀ alkylene is unsubstituted, and is, for instance, an unsubstituted C₁₋₄ alkylene group, for example ethylene. Typically, when R⁵ is substituted or unsubstituted C₁₋₂₀ alkylene-O—C₃₋₂₀ heterocyclyl, said C₃₋₂₀ heterocyclyl is a group of the following formula (m):

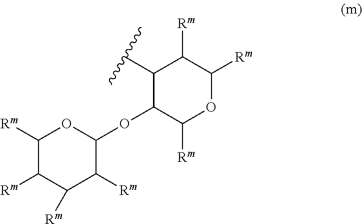

wherein each R^{m}, which is the same or different, is independently selected from C₁₋₆ alkyl, OH, acyloxy, SH, C₁₋₆ alkoxy, aryloxy, amino, C₁₋₁₀ alkylamino, di(C₁₋₁₀)alkylamino, amido and acylamido. More typically, when R⁵ is substituted or unsubstituted C₁₋₂₀ alkylene-O—C₃₋₂₀ heterocyclyl, said C₃₋₂₀ heterocyclyl is a group of the following structure:

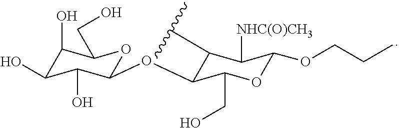

Alternatively, R⁵ may be substituted or unsubstituted aryl, substituted or unsubstituted C₃₋₂₀ heteroaryl, substituted or unsubstituted C₃₋₂₀ heterocyclyl or substituted or unsubstituted C₃₋₂₅ cycloalkyl. Thus, R⁵ may be substituted or unsubstituted phenyl or substituted or unsubstituted cyclohexyl, for example.

In the compounds of formula (I), typically X is NR⁵, and R⁵ forms, together with R⁴ or R¹⁴ (typically R⁴), a substituted or unsubstituted C₁₋₆ alkylene group, or R⁵ is selected from hydrogen, unsubstituted or substituted C₁₋₂₀ alkyl which is optionally interrupted by 0, and a group of the following formula (VIII)

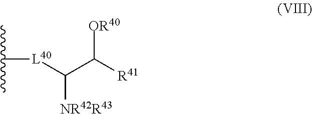

in which:

R⁴⁰ and R⁴², which are the same or different, are independently selected from H, substituted or unsubstituted C₁₋₆ alkyl or substituted or unsubstituted phenyl;

R⁴¹ is H, substituted or unsubstituted aryl, —CH═CHR⁴⁴, or substituted or unsubstituted C₁₋₂₀ alkyl, which C₁₋₂₀ alkyl is optionally interrupted by N(R′), O, S or arylene wherein R′ is H, C₁₋₆ alkyl or aryl;

R⁴³ is H, substituted or unsubstituted C₁₋₆ alkyl, substituted or unsubstituted phenyl or —C(O)R⁴⁷;

R⁴⁴ is H or substituted or unsubstituted C₁₋₂₀ alkyl, which C₁₋₂₀ alkyl is optionally interrupted by N(R′), O, S or arylene;

R⁴⁷ is substituted or unsubstituted C₁₋₂₀ alkyl, which C₁₋₂₀ alkyl is optionally interrupted by N(R′), O, S or arylene; and

L⁴⁰ is substituted or unsubstituted C₁₋₁₀ alkylene.

Typically, R⁴⁰ is H. Typically, R⁴² is H. Typically, R⁴³ is H or —C(O)R⁴⁷. More typically, R⁴³ is —C(O)R⁴⁷. Typically, R⁴⁷ is unsubstituted C₁₋₂₀ alkyl. R⁴⁷ may be, for instance, C₇H₁₅ (as in eliglustat), C₉H₁₉ (as in PDMP) or C₁₅H₃₁ (as in the P4 compounds). Typically L⁴⁰ is CH₂. In one embodiment, R⁴¹ is —CH═CHR⁴⁴ and R⁴⁴ is unsubstituted C₁₋₂₀ alkyl. In that embodiment, R⁴⁴ may be, for instance, —C₁₃H₂₇. In another embodiment, R⁴¹ is a group of the following formula (VIIIa):

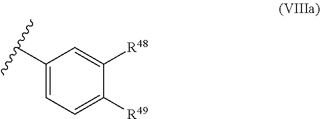

in which R⁴⁸ is H, C₁₋₆ alkyl, phenyl or, together with R⁴⁹ a bidentate group of the structure —O-alk-O—; R⁴⁹ is H, C₁₋₆ alkyl, phenyl or, together with R⁴⁸ a bidentate group of the structure —O-alk-O—, wherein alk is substituted or unsubstituted C₁₋₆ alkylene. Typically, R⁴⁸ is H or, together with R⁴⁹ a bidentate group of the structure —O—CH₂—CH₂—O—. Typically, R⁴⁹ is H, OH or, together with R⁴⁸ a bidentate group of the structure —O—CH₂—CH₂—O—. Typically, R⁴⁸ is H and R⁴⁹ is either H or OH.

Typically, when R⁵ is C₁₋₂₀ alkyl optionally interrupted by O, R⁵ is methyl, ethyl, propyl, butyl, pentyl, hexyl, heptyl, octyl, nonyl, decyl, methyl-O—R⁹⁰, ethyl-O—R⁹⁰, propyl-O—R⁹⁰, butyl-O—R⁹⁰, pentyl-O—R⁹⁰, hexyl-O—R⁹⁰, heptyl-O—R⁹⁰, octyl-O—R⁹⁰, nonyl-O—R⁹⁰ or decyl-O—R⁹⁰ wherein R⁹⁰ is methyl, ethyl, propyl, butyl, pentyl, hexyl, heptyl, octyl, nonyl, decyl or adamantyl.

Typically, when R⁵ forms, together with R⁴ or R¹⁴, a substituted or unsubstituted C₁₋₆ alkylene group, the alkylene group is substituted or unsubstituted propylene. Typically, said propylene is unsubstituted or substituted with a C₁₋₄ alkyl group, for instance with a methyl group. Examples of compounds of formula (I) in which R⁴ or R¹⁴ forms, together with R⁵, a methyl-substituted propylene group are Castanospermine and MDL25874, whose structures are given below.

Alternatively, X is O or S. More typically, X is O.

Typically, R¹², R¹³, R¹⁴ and R¹⁶ are all H and the inhibitor of glycosphingolipid biosynthesis is of formula (Ia) below:

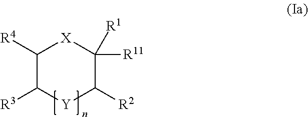

wherein X is O, S or NR⁵; Y is O, S or CHR⁶; n is 0 or 1; and R¹, R², R³, R⁴ R⁵, R⁶ and R¹¹ are as defined above for formula (I).

In one embodiment, the inhibitor of glycosphingolipid biosynthesis is of formula (Ia) and: X is NR⁵; n is 1; Y is CHR⁶; and R⁵ is selected from: hydrogen and unsubstituted or substituted C₁₋₂₀ alkyl which is optionally interrupted by O, or R⁵ forms, together with R⁴, a substituted or unsubstituted C₁₋₆ alkylene group.

Typically in this embodiment, R¹¹ is H. Typically in this embodiment, R¹, R², R³ and R⁶, which may be the same or different, are independently selected from H, OH, acyloxy, and substituted or unsubstituted C₁₋₆ alkyl. When said C₁₋₆ alkyl is substituted, it is typically substituted with 1, 2, 3 or 4 groups selected from hydroxyl and acyloxy. Typically, in this embodiment, R⁴ is either C₁₋₆ alkyl substituted with 1, 2, 3 or 4 groups selected from hydroxyl and acyloxy, or R⁴ forms, together with R⁵, a substituted or unsubstituted C₁₋₆ alkylene group. For instance, R⁴ may be methyl, ethyl, propyl or butyl substituted with 1, 2, 3 or 4 groups respectively, which groups are selected from hydroxyl and acyloxy, more typically hydroxyl. R⁴ may be CH₂OH. Alternatively R⁴ may be a group which, together with R⁵, is a substituted or unsubstituted propylene group. Typically, in this embodiment, R², R³ and R⁶ are all OH. Typically, in this embodiment R¹ is selected from H, OH and C₁₋₆ alkyl which is unsubstituted or substituted with one, two, three or four groups selected from hydroxyl and acyloxy. For instance, R¹ may be H, OH, unsubstituted C₁₋₆ alkyl, methyl, ethyl, propyl or butyl, which methyl, ethyl, propyl and butyl are substituted with 1, 2, 3 or 4 groups respectively, which groups are selected from hydroxyl and acyloxy, more typically hydroxyl. More typically, R⁴ is H, OH, CH₂OH or C₁₋₆ alkyl. In this embodiment, the modification of the iminosugar core with an N-alkyl chain such as a N-butyl group (as in NB-DNJ) or a N-nonyl group (as in NN-DNJ) is believed to be important for clinical applications. Thus, typically in this embodiment R⁵ is C₁₋₂₀ alkyl which is optionally interrupted by O. For instance, R⁵ may be methyl, ethyl, propyl, butyl, pentyl, hexyl, heptyl, octyl, nonyl, decyl, methyl-O—R⁹⁰, ethyl-O—R⁹⁰, propyl-O—R⁹⁰, butyl-O—R⁹⁰, pentyl-O—R⁹⁰, hexyl-O—R⁹⁰, heptyl-O—R⁹⁰, octyl-O—R⁹⁰, nonyl-O—R⁹⁰ or decyl-O—R⁹⁰ wherein R⁹⁰ is methyl, ethyl, propyl, butyl, pentyl, hexyl, heptyl, octyl, nonyl, decyl or adamantyl. Alternatively, R⁵, together with R⁴ is a substituted or unsubstituted Ci-6 alkylene group. Typically, the alkylene group is substituted or unsubstituted propylene. Typically, said propylene is unsubstituted or substituted with a C₁₋₄ alkyl group, for instance with a methyl group. Alternatively, R⁵ may be H. Typically, when R² is acyloxy, said acyloxy is selected from —OC(O)CH₃, —OC(O)CH₂CH₃, —OC(O)CH₂CH₂CH₃ and —OC(O)CH₂CH₂CH₂CH₃. More typically, when R² is acyloxy, said acyloxy is —OC(O)CH₂CH₂CH₃. Alternatively, R⁵ is substituted or unsubstituted C₁₋₂₀ alkylene-O—C₃₋₂₀ heterocyclyl. More typically, R⁵ is C₁₋₄ alkylene-O—C₃₋₂₀ heterocyclyl, wherein said C₁₋₄ alkylene is unsubstituted and said C₃₋₂₀ heterocyclyl is a group of the following formula (m):

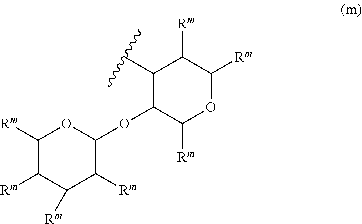

wherein each R^{m}, which are the same or different, is independently selected from C₁₋₆ alkyl, OH, acyloxy, SH, C₁₋₆ alkoxy, aryloxy, amino, C₁₋₁₀ alkylamino, di(C₁₋₁₀)alkylamino, amido and acylamido. Even more typically, R⁵ is C₁₋₄ alkylene-O—C₃₋₂₀ heterocyclyl, wherein said C₁₋₄ alkylene is unsubstituted and said C₃₋₂₀ heterocyclyl is a group of the following structure:

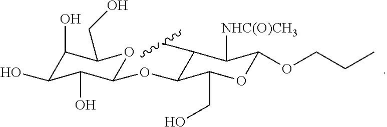

In another embodiment, the inhibitor of glycosphingolipid biosynthesis is of formula (Ia) and: X is NR⁵; Y is O or S; n is either 0 or 1; and R⁵ is selected from hydrogen and a group of the following formula (VIII):

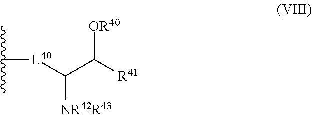

in which R⁴⁰ and R⁴², which are the same or different, are independently selected from H, substituted or unsubstituted C₁₋₆ alkyl or substituted or unsubstituted phenyl; R⁴¹ is H, substituted or unsubstituted aryl, —CH═CHR⁴⁴, or substituted or unsubstituted C₁₋₂₀ alkyl, which C₁₋₂₀ alkyl is optionally interrupted by N(R′), O, S or arylene wherein R′ is H, C₁₋₆ alkyl or aryl; R⁴³ is H, substituted or unsubstituted C₁₋₆ alkyl, substituted or unsubstituted phenyl or —C(O)R⁴⁷; R⁴⁴ is H or substituted or unsubstituted C₁₋₂₀ alkyl, which C₁₋₂₀ alkyl is optionally interrupted by N(R′), O, S or arylene; R⁴⁷ is substituted or unsubstituted C₁₋₂₀ alkyl, which C₁₋₂₀ alkyl is optionally interrupted by N(R′), O, S or arylene; and L⁴⁰ is substituted or unsubstituted C₁₋₁₀alkylene. Typically, R⁴⁰ is H. Typically, R⁴² is H. Typically, R⁴³ is H or —C(O)R⁴⁷. More typically, R⁴³ is —C(O)R⁴⁷. Typically, R⁴⁷ is unsubstituted C₁₋₂₀ alkyl. R⁴⁷ may be, for instance, C₇H₁₅ (as in eliglustat), C₉H₁₉ (as in PDMP) or C₁₅H₃₁ (as in the P4 compounds). Typically L⁴⁰ is CH₂. In one embodiment, R⁴¹ is —CH═CHR⁴⁴ and R⁴⁴ is unsubstituted C₁₋₂₀ alkyl. In that embodiment, R⁴⁴ may be, for instance, —C₁₃H₂₇. Alternatively, R⁴¹ is a group of the following formula (VIIIa):

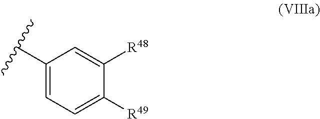

in which R⁴⁸ is H, C₁₋₆ alkyl, phenyl or, together with R⁴⁹ a bidentate group of the structure —O-alk-O—; R⁴⁹ is H, C₁₋₆ alkyl, phenyl or, together with R⁴⁸ a bidentate group of the structure —O-alk-O—, wherein alk is substituted or unsubstituted C₁₋₆ alkylene. Typically, R⁴⁸ is H or, together with R⁴⁹ a bidentate group of the structure —O—CH₂—CH₂—O—. Typically, R⁴⁹ is H, OH or, together with R⁴⁸ a bidentate group of the structure —O—CH₂—CH₂—O—. Typically, R⁴⁸ is H and R⁴⁹ is either H or OH. Typically in this embodiment, R¹¹ is H. Typically in this embodiment, R¹, R², R³, R⁴ and R⁶, which may be the same or different, are independently selected from H, OH, acyloxy and C₁₋₆ alkyl which is unsubstituted or substituted with one, two, three or four groups selected from hydroxyl and acyloxy. More typically, in this embodiment, R¹, R², R³, R⁴ and R⁶ are independently selected from H, OH and CH₂OH. Typically, in this embodiment, Y is O. Examples of compounds of this embodiment include D,L-threo-1-phenyl-2-decanoylamino-3-morpholino-1-propanol (PDMP); D,L-threo-1-phenyl-2-hexadecanoylamino-3-morpholino-1-propanol (PPMP); D-threo-1-phenyl-2-palmitoilamino-3-pyrrolidino-1-propanol (P4); 4′-hydroxy-D-threo-1-phenyl-2-palmitoilamino-3-pyrrolidino-1-propanol (4′-hydroxy-P4); 3′,4′-ethylenedioxy-P4 (EtDO-P4); N-[(1R,2R)-1-(2,3-Dihydro-1,4-benzodioxin-6-yl)-1-hydroxy-3-(1-pyrrolidinyl)-2-propanyl] octanamide (eliglustat) and 2,5-dihydroxymethyl-3,4-dihydroxypyrrolidine (L-DMDP).

In another embodiment, the inhibitor of glycosphingolipid biosynthesis is of formula (Ia) and: X is O or S; n is 1; Y is CHR⁶; R⁶ is H, hydroxyl, acyloxy, C₁₋₂₀ alkoxy, C₁₋₁₀alkylamino or di(C₁₋₁₀)alkylamino; R¹¹ is H; R² and R³, which may be the same or different, are independently selected from H, hydroxyl, C₁₋₂₀ alkoxy, acyloxy or acylamido; R⁴ is H, hydroxyl, acyloxy, thiol or C₁₋₂₀ alkyl which is unsubstituted or substituted with one, two, three or four groups selected from hydroxyl, acyloxy and thiol; and R¹ is C₁₋₂₀ alkoxy, aryloxy or —O—C₃₋₂₀ heterocyclyl, wherein said heterocyclyl is a group derived from a group of the following structure:

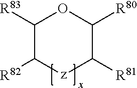

wherein x is 0 or 1; z is CHR⁸⁴; and R⁸⁰, R⁸¹, R⁸², R⁸³ and R⁸⁴, which are the same or different, are independently selected from H, C₁₋₆ alkyl, OH, acyloxy, SH, C₁₋₆ alkoxy, aryloxy, amino, C₁₋₁₀ alkylamino, di(C₁₋₁₀)alkylamino, amido and acylamido. Typically, in this embodiment, X is O. Examples of compounds of this embodiment are the Galactosyltransferase inhibitor compounds described in Chung S J, Bioorg Med Chem Lett. 1998 Dec. 1; 8(23):3359-64, whose structures are given hereinbelow.

Typically, in this embodiment, R¹ is a group of any one of the following structures:

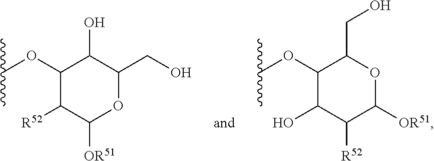

in which R⁵¹ is a substituted or unsubstituted C₁₋₁₀alkyl group, typically methyl, or a substituted or unsubstituted aryl group, typically a phenyl or naphthyl group. The phenyl or naphthyl may be unsubstituted or substituted. When substituted, the phenyl or naphthyl is typically substituted with a halo group, for instance with a bromo group. R⁵² is typically hydroxyl, C₁₋₁₀alkoxy, acyloxy, aryloxy or acylamido. Typically, R⁵² is —OH or —NHC(O)Me.

Alternatively, in this embodiment, R¹ may be C₁₋₂₀ alkoxy wherein said C₁₋₂₀ alkoxy group is substituted with an ester group or an aryl group, for instance with —C(O)OCH₃ or Ph. Alternatively, in this embodiment, R¹ may be aryloxy wherein the aryl group bonded to the oxygen of said aryloxy is either substituted or unsubstituted phenyl, or substituted or unsubstituted naphthyl. Typically, the phenyl or naphthyl is either unsubstituted or monosubstituted with halo or methoxy.

Typically, in this embodiment, R⁶ is H, amino or hydroxyl, more typically, amino or hydroxyl. Typically, in this embodiment, R² is H, hydroxyl or —NHC(O)CH₃, more typically hydroxyl or —NHC(O)CH₃. Typically, in this embodiment, R³ is H or hydroxyl, more typically hydroxyl. Typically, in this embodiment, R⁴ is H, CH₂OH or CH₂SH, more typically CH₂OH or CH₂SH.

In another embodiment, the inhibitor of glycosphingolipid biosynthesis is of formula (Ia) and:

X is O or S; n is 1; Y is CHR⁶; R⁶ is H, hydroxyl, acyloxy or C₁₋₂₀ alkoxy;

R¹ and R¹¹ which may be the same or different, are independently selected from H, C₁₋₂₀ alkyl, hydroxyl, acyloxy, C₁₋₂₀ alkoxy, carboxyl, ester, —O—C₃₋₂₅ cycloalkyl, and a group of the following formula (VII):

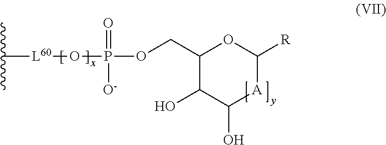

wherein L⁶⁰ is substituted or unsubstituted C₁₋₂₀ alkylene; x is 0 or 1; y is 0 or 1; A is CHR′″ and R is H, C₁₋₂₀ alkyl, C₃₋₂₀ heterocyclyl, C₃₋₂₅ cycloalkyl, aryl or C₁₋₂₀ alkoxy, wherein R′″ is hydroxyl, C₁₋₆ alkoxy, aryloxy or acyl;

R² is H, C₁₋₂₀ alkyl, hydroxyl, acyloxy or —O—C₃₋₂₀ heterocyclyl, wherein said heterocyclyl is a group derived from a group of the following structure:

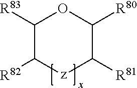

wherein x is 0 or 1; z is CHR⁸⁴; and R⁸⁰, R⁸¹, R⁸², R⁸³ and R⁸⁴, which are the same or different, are independently selected from H, C₁₋₆ alkyl, OH, SH, C1-6 alkoxy, aryloxy, amino, C₁₋₁₀ alkylamino, di(C₁₋₁₀)alkylamino, amido, acylamido and a group derived from a second group of the following structure:

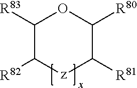

in which second group x is 0 or 1; z is CHR⁸⁴; and R⁸⁰, R⁸¹, R⁸², R⁸³ and R⁸⁴, which are the same or different, are independently selected from H, C₁₋₆ alkyl, OH, SH, C₁₋₆ alkoxy, aryloxy, amino, C₁₋₁₀ alkylamino, di(C₁₋₁₀)alkylamino, amido and acylamido;

R³ is H, hydroxyl, acyloxy, C₁₋₂₀ alkoxy or acylamido; and

R⁴ is H, carboxyl, ester or C₁₋₂₀ alkyl which is unsubstituted or substituted with one, two, three or four groups selected from hydroxyl and thiol.

Examples of compounds of this embodiment are sialic acid, cytidin-5′-yl sialylethylphosphonate and Soyasaponin I.

Typically, in this embodiment, X is O.

Typically, in this embodiment, R⁶ is H or hydroxyl, more typically hydroxyl.

Typically, in this embodiment, R¹ and R¹¹ are independently selected from H, hydroxyl, carboxyl, —O—C₃₋₂₅ cycloalkyl and a group of formula (VII) in which L⁶⁰ is ethylene or methylene, R′″ is hydroxyl, and R is either —OCH₃ or a heterocyclic group of the following structure:

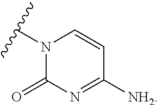 [SMILES: *n1ccc(N)nc1=O]

Both R¹ and R¹¹ may be groups of formula (VII).

When R¹ or R¹¹ is —O—C₃₋₂₅ cycloalkyl, the cycloalkyl group is a group derived from a compound of one of the following formulae, which compound may be substituted or unsubstituted:

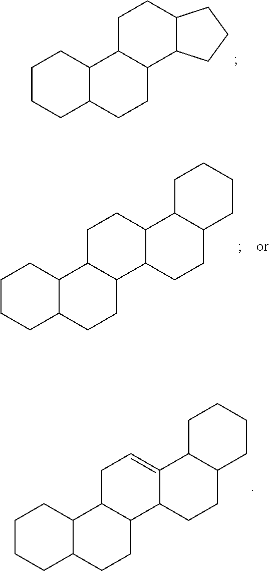

More typically, the cycloalkyl group of said —O—C₃₋₂₅ cycloalkyl is a group derived from the following compound:

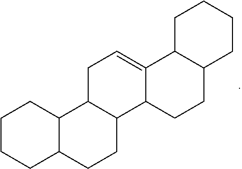 [SMILES: C1=C2C3CCCCC3CCC2C2CCC3CCCCC3C2C1]

Typically, if either R¹ or R¹¹ is —O—C₃₋₂₅ cycloalkyl, then the other one of those groups, i.e. R¹¹ or R¹ respectively, is H.

Typically, in this embodiment, R² is H or —O—C₃₋₂₀ heterocyclyl, wherein said heterocyclyl is a group of the following structure:

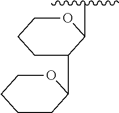 [SMILES: *[C@H]1OCCC[C@@H]1[C@@H]1CCCCO1]

wherein each of the ring carbon atoms is independently unsubstituted or substituted with C₁₋₆ alkyl, OH, SH, C₁₋₆ alkoxy, aryloxy, amino, C₁₋₁₀ alkylamino, di(C₁₋₁₀)alkylamino, amido and acylamido. Typically, each of the ring carbon atoms is independently unsubstituted or substituted with OH, CH₂OH or a C₁₋₆ alkyl group, for instance a methyl group.

Typically, in this embodiment, R³ is hydroxyl or acylamido. More typically, R³ is hydroxyl or NHC(O)CH₃.

Typically, in this embodiment, R⁴ is carboxyl, methyl, ethyl, propyl or butyl, which methyl, ethyl, propyl or butyl are substituted with one, two, three and four groups respectively, which groups are selected from hydroxyl and thiol. More typically, R⁴ is carboxyl or —CH(OH)CH(OH)CH₂OH.

Any of the following compounds of formula (I), and any pharmaceutically acceptable salts of the following compounds of formula (I), may be employed in the present invention:
Iminosugars (azasugars) such as: N-butyldeoxynojirimycin (NB-DNJ), also known as miglustat or ZAVESCA®; N-nonyldeoxynojirimycin (NN-DNJ); N-butyldeoxygalactonojirimycin (NB-DGJ); N-5-adamantane-1-yl-methoxypentyl-deoxynojirimycin (AMP-DNJ); alpha-homogalactonojirimycin (HGJ); Nojirimycin (NJ); Deoxynojirimycin (DNJ); N7-oxadecyl-deoxynojirimycin; deoxygalactonojirimycin (DGJ); N-butyl-deoxygalactonojirimycin (NB-DGJ); N-nonyl-deoxygalactonojirimycin (NN-DGJ); N-nonyl-6deoxygalactonojirimycin; N7-oxanonyl-6deoxy-DGJ; alpha-homoallonojirimycin (HAJ); beta-1-C-butyl-deoxygalactonojirimycin (CB-DGJ). Such compounds are glycosyltransferase inhibitors (“sugar mimics”).
D,L-threo-1-phenyl-2-decanoylamino-3-morpholino-1-propanol (PDMP); D,L-threo-1-phenyl-2-hexadecanoylamino-3-morpholino-1-propanol (PPMP); D-threo-1-phenyl-2-palmitoilamino-3-pyrrolidino-1-propanol (P4); 4′-hydroxy-D-threo-1-phenyl-2-palmitoilamino-3-pyrrolidino-1-propanol (4′-hydroxy-P4); 3′,4′-ethylenedioxy-P4 (EtDO-P4); N-[(1R,2R)-1-(2,3-Dihydro-1,4-benzodioxin-6-yl)-1-hydroxy-3-(1-pyrrolidinyl)-2-propanyl]octanamide (eliglustat); 2,5-dihydroxymethyl-3,4-dihydroxypyrrolidine (L-DMDP). Such compounds are glycosyltransferase inhibitors and derivatives of sphingosine (“lipid mimics”).
Iminosugars such as Castanospermine and MDL25874, which have the following structures respectively:

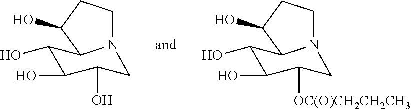

Sialyltransferase inhibitors such as N-acetylneuraminic acid (sialic acid); cytidin-5′-yl sialylethylphosphonate; and Soyasaponin I.
Galactosyltransferase inhibitor compounds of the following structures, which compounds are described in Chung S J, Bioorg Med Chem Lett. 1998 Dec. 1; 8(23):3359-64:

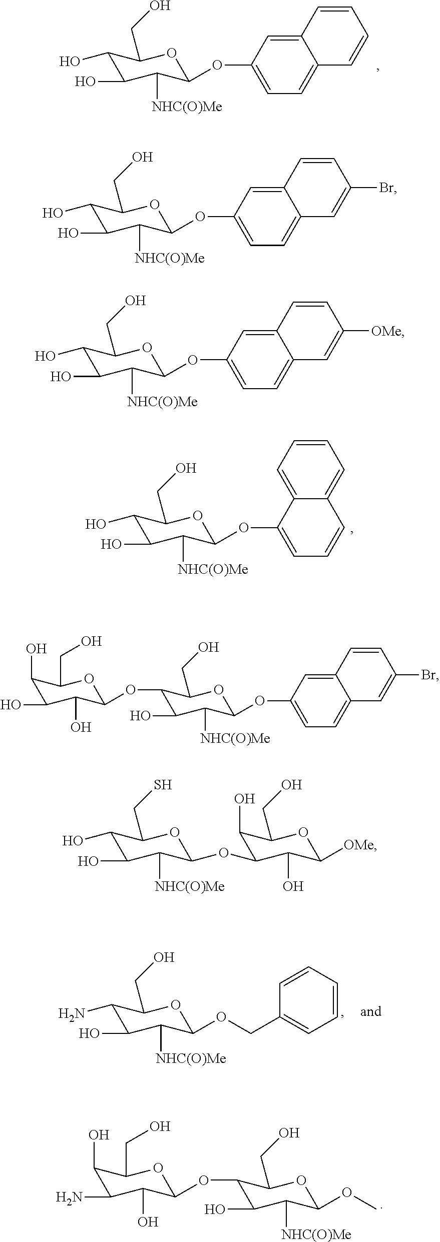

Iminosugars, such as 1,5-dideoxy-1,5-imino-D-glucitol, and their N-alkyl, N-acyl and N-aryl, and optionally O-acylated derivatives, such as: 1,5-(Butylimino)-1,5-dideoxy-D-glucitol, also known as N-butyldeoxynojirimycin (NB-DNJ), miglustat or ZAVESCA®; 1,5-(Methylimino)-1,5-dideoxy-D-glucitol; 1,5-(Hexylimino)-1,5-dideoxy-D-glucitol; 1,5-(Nonylylimino)-1,5-dideoxy-D-glucitol; 1,5-(2-Ethylbutylimino)-1,5-dideoxy-D-glucitol; 1,5-(2-Methylpentylimino)-1,5-dideoxy-D-glucitol; 1,5-(Benzyloxycarbonylimino)-1,5-dideoxy-D-glucitol, tetraacetate; 1,5-(Phenylacetylimino)-1,5-dideoxy-D-glucitol, tetraacetate; 1,5-(Benzoylimino)-1,5-dideoxy-D-glucitol, tetraacetate; 1,5-(Butylimino)-1,5-dideoxy-D-glucitol, tetraacetate; 1,5-(Ethyl malonylimino)-1,5-dideoxy-D-glucitol, tetraacetate; 1,5-(Hexylimino)-1,5-dideoxy-D-glucitol, tetraacetate; 1,5-(Nonylimino)-1,5-dideoxy-D-glucitol, tetraacetate; 1,5-(Benzyloxycarbonylimino)-1,5-dideoxy-D-glucitol, tetraisobutyrate; 1,5-(Butylimino)-1,5-dideoxy-D-glucitol, tetrabutyrate; 1,5-(Butylimino)-1,5-dideoxy-D-glucitol, tetrapropionate; 1,5-(Butylimino)-1,5-dideoxy-D-glucitol, tetrabenzoate; 1,5-Dideoxy-1,5-imino-D-glucitol, tetraisobutyrate; 1,5-(Hydrocinnamoylimino)-1,5-dideoxy-D-glucitol, tetraacetate; 1,5-(Methyl malonylimino)-1,5-dideoxy-D-glucitol, tetraacetate; 1,5-(Butylimino)-1,5-dideoxy-D-glucitol, tetraisobutyrate; 1,5-(Butylimino)-1,5-dideoxy-4R,6-O-(phenylmethylene)-D-glucitol, diacetate; 1,5-[(Phenoxymethyl)carbonylimino]-1,5-dideoxy-D-glucitol, tetraacetate; 1,5-[(Ethylbutyl)imino]-1,5-dideoxy-D-glucitol, tetraacetate; 1,5-(Butylimino)-1,5-dideoxy-D-glucitol, 2,3-diacetate; 1,5-(Hexylimino)-1,5-dideoxy-4R,6-O-(phenylmethylene)-D-glucitol, diacetate; 1,5-(Hexylimino)-1,5-dideoxy-D-glucitol, 2,3-diacetate; 1,5-[(2-Methylpentyl)imino]-1,5-dideoxy-D-glucitol, tetraacetate; 1,5-(Butylimino)-1,5-dideoxy-D-glucitol, 6-acetate; 1,5-[(3-Nicotinoyl)imino]-1,5-dideoxy-D-glucitol, tetraacetate; 1,5-(Cinnamoylimino)-1,5-dideoxy-D-glucitol, tetraacetate; 1,5-(Butylimino)-1,5-dideoxy-D-glucitol, 2,3-dibutyrate; 1,5-(Butylimino)-1,5-dideoxy-4R,6-O-(phenylmethylene)-D-glucitol, 2,3-dibutyrate; 1,5-(Phenylacetylimino)-1,5-dideoxy-D-glucitol, tetraisobutyrate; 1,5-[(4-Chlorophenyl)acetylimino]-1,5-dideoxy-D-glucitol, tetraacetate; 1,5-[(4-Biphenyl)acetylimino]-1,5-dideoxy-D-glucitol, tetraacetate; 1,5-(Benzyloxycarbonylimino)-1,5-dideoxy-D-glucitol, tetrabutyrate; 1,5-Dideoxy-1,5-imino-D-glucitol, tetrabutyrate; 3,4,5-piperidinetriol, 1-propyl-2-(hydroxymethyl)-, (2S, 3R, 4R, 5S); 3,4,5-piperidinetriol, 1-pentyl-2-(hydroxymethyl)-, (2S, 3R, 4R, 5S); 3,4,5-piperidinetriol, 1-heptyl-2-(hydroxymethyl)-, (2S, 3R, 4R, 5S); 3,4,5-piperidinetriol, 1-butyl-2-(hydroxymethyl)-, (2S, 3S, 4R, 5S); 3,4,5-piperidinetriol, 1-nonyl-2-(hydroxymethyl)-, (2S, 3R, 4R, 5S); 3,4,5-piperidinetriol, 1-(1-ethyl) propyl-2-(hydroxymethyl)-, (2S, 3R, 4R, 5S); 3,4,5-piperidinetriol, 1-(3-methyl) butyl-2-(hydroxymethyl)-, (2S, 3R, 4R, 5S); 3,4,5-piperidinetriol, 1-(2-phenyl) ethyl-2-(hydroxymethyl)-, (2S, 3R, 4R, 5S); 3,4,5-piperidinetriol, 1-(3-phenyl) propyl-2-(hydroxymethyl)-, (2S, 3R, 4R, 5S); 3,4,5-piperidinetriol, 1-(1-ethyl) hexyl-2-(hydroxymethyl)-, (2S, 3R, 4R, 5S); 3,4,5-piperidinetriol, 1-(2-ethyl) butyl-2-(hydroxymethyl)-, (2S, 3R, 4R, 5S); 3,4,5-piperidinetriol, 1-[(2R)-(2-methyl-2-phenyl) ethyl]-2-(hydroxymethyl)-, (2S, 3R, 4R, 5S); 3,4,5-piperidinetriol, 1-[(2S)-(2-methyl-2-phenyl) ethyl]-2-(hydroxymethyl)-, (2S, 3R, 4R, 5S), β-L-homofuconojirimycin; and propyl 2-acetamido-2-deoxy-4-O-(β-D-galactopyranosyl)-3-O-(2-(N—(β-L-homofuconojirimycinyl))ethyl)-α-D-glucopyranoside.
Iminosugars, such as ido-N-(5-adamantane-1-yl-methoxy-pentyl)deoxynojirimycin; N-(adamantane-1-yl-methoxypentyl)-L-ido-deoxynojirimycin; N-(adamantane-1-yl-methoxypentyl)-D-galacto-deoxynojirimycin; C1-beta-(adamantane-1-yl-methoxypentyl)-deoxynojirimycin; N-methyl-C1-beta-(adamantane-1-yl-methoxypentyl)-deoxynojirimycin; N-butyl-C1-beta-(adamantane-1-yl-methoxypentyl)-deoxynojirimycin; 2-O-(adamantane-1-yl-methoxypentyl)-deoxynojirimycin; N-methyl-2-O-(adamantane-1-yl-methoxy-pentyl)-deoxynojirimycin; N-butyl-2-O-(adamantane-1-yl-methoxy-pentyl)-deoxynojirimycin; N-benzyloxycarbonyl-2-O-(adamantane-1-yl-methoxypentyl)-3,4,6-tri-O-benzyl-deoxy-nojirimycin; and N-(5-adamantane-1-yl-methoxy-pentyl)deoxynojirimycin.

Methods of synthesizing such iminosugar compounds are known and are described, for example, in WO 02/055498 and in U.S. Pat. Nos. 5,622,972, 4,246,345, 4,266,025, 4,405,714, and 4,806,650, U.S. patent application Ser. No. 07/851,818, filed Mar. 16, 1992, US 2007/0066581 and EP1528056. For example, N-nonyl-DNJ and N-decyl-DNJ can be conveniently prepared by the N-nonylation or N-decylation, respectively, of 1,5-dideoxy-1,5-imino-D-glucitol (DNJ) by methods analogous to the N-butylation of DNJ as described in Example 2 of U.S. Pat. No. 4,639,436 by substituting an equivalent amount of n-nonylaldehyde or n-decylaldehyde for n-butylraldehyde. The starting materials are readily available from many commercial sources.

Typically, the compound of formula (I) employed is N-butyldeoxynojirimycin (NB-DNJ) or N-butyldeoxygalactonojirimycin (NB-DGJ). More typically, the compound of formula (I) is NB-DNJ.

NB-DGJ is the galactose analogue of NB-DNJ. NB-DGJ inhibits GSL biosynthesis comparably to NB-DNJ but lacks certain side effect activities associated with NB-DNJ. There has been extensive use of NB-DGJ in mouse models of GSL storage diseases and it is very well tolerated. Thus, in one embodiment, the compound of formula (I) employed is NB-DGJ.

In one preferred embodiment, the compound of formula (I) employed is selected from: ido-N-(5-adamantane-1-yl-methoxy-pentyl)deoxynojirimycin; N-(adamantane-1-yl-methoxypentyl)-L-ido-deoxynojirimycin; N-(adamantane-1-yl-methoxypentyl)-D-galacto-deoxynojirimycin; C1-beta-(adamantane-1-yl-methoxypentyl)-deoxynojirimycin; N-methyl-C1-beta-(adamantane-1-yl-methoxypentyl)-deoxynojirimycin; N-butyl-C1-beta-(adamantane-1-yl-methoxypentyl)-deoxynojirimycin; 2-O-(adamantane-1-yl-methoxypentyl)-deoxynojirimycin; N-methyl-2-O-(adamantane-1-yl-methoxy-pentyl)-deoxynojirimycin; N-butyl-2-O-(adamantane-1-yl-methoxy-pentyl)-deoxynojirimycin; N-benzyloxycarbonyl-2-O-(adamantane-1-yl-methoxypentyl)-3,4,6-tri-O-benzyl-deoxy-nojirimycin; and N-(5-adamantane-1-yl-methoxy-pentyl)deoxynojirimycin.

In another preferred embodiment the compound of formula (I) employed is selected from D,L-threo-1-phenyl-2-decanoylamino-3-morpholino-1-propanol (PDMP); D,L-threo-1-phenyl-2-hexadecanoylamino-3-morpholino-1-propanol (PPMP); D-threo-1-phenyl-2-palmitoilamino-3-pyrrolidino-1-propanol (P4); 4′-hydroxy-D-threo-1-phenyl-2-palmitoilamino-3-pyrrolidino-1-propanol (4′-hydroxy-P4); 3′,4′-ethylenedioxy-P4 (EtDO-P4); N-[(1R,2R)-1-(2,3-Dihydro-1,4-benzodioxin-6-yl)-1-hydroxy-3-(1-pyrrolidinyl)-2-propanyl]octanamide (eliglustat); and 2,5-dihydroxymethyl-3,4-dihydroxypyrrolidine (L-DMDP). Typically, in this embodiment, the compound of formula (I) employed is N-[(1R,2R)-1-(2,3-Dihydro-1,4-benzodioxin-6-yl)-1-hydroxy-3-(1-pyrrolidinyl)-2-propanyl]octanamide (eliglustat). Eliglustat may be employed in the form of a pharmaceutically acceptable salt of eliglustat, for instance in the form of eliglustat tartrate.

### Infection by a Pathogenic Mycobacterium

Tuberculosis is a global health problem affecting approximately one third of the world's population and resulting in three million deaths each year. One of the characteristic hallmarks of tuberculosis is the ability of Mtb to successfully survive within cells of the innate immune system including macrophages and monocytes.

Non-pathogenic mycobacteria, including Mycobacterium smegmatis, bind host cell-surface receptors and are ingested into phagosomes that subsequently mature and fuse with lysosomes, leading to destruction of the mycobacterium.

In contrast, pathogenic mycobacteria are not delivered to the lysosome due to the mycobacterium inhibiting phagosome-lysosome fusion. Multiple mechanisms have been proposed to explain the intracellular survival of Mtb, including phagosome maturation arrest, and defective acidification of the phagosome and inhibition of phosphatidylinositol-dependent trafficking pathways.

The present inventors have investigated whether there is a mechanistic link between pathogenic mycobacterial infection and the NPC pathway. The inventors unexpectedly found that pathogenic mycobacteria, including Mycobacterium tuberculosis, secrete lipids that inhibit the NPC pathway, promoting the intra-cellular survival of the mycobacteria in host macrophages. This link between the rare lysosomal storage disorder NPC and Mtb infection has important implications for understanding host-pathogen interactions and for developing new therapies to combat TB, particularly in the current era of antibiotic resistance.

The inventors found that treatment with a particular class of pharmacological agent that corrects defects in NPC1 mutant cells, namely a SERCA antagonist, promotes mycobacterial clearance. Other classes of agent known to be useful against NPC disease, however, did not promote mycobacterial clearance by themselves, including an inhibitor of glycosphingolipid biosynthesis miglustat (NB-DNJ), and β-cyclodextrin. Furthermore, a structural analog of curcumin that does not function as SERCA antagonist was found not to promote clearance of the mycobacterium. The data herein support that the lipids secreted by the pathogenic mycobacteria inhibit the NPC pathway to achieve altered acidic store calcium homeostasis, that in turn blocks the ability of the lysosome to fuse and kill the mycobacterium, leading to persistence.

Despite the fact that the inhibitor of glycosphingolipid biosynthesis was found to be inactive on its own within the time frame of the experiment, it was unexpectedly found that inhibitors of glycosphingolipid biosynthesis show synergy when combined with a SERCA antagonist. Indeed, the combination of a SERCA antagonist (which elevates cytosolic calcium) and an inhibitor of glycosphingolipid biosynthesis showed significantly greater efficacy than the SERCA antagonist on its own and, of course, than the inhibitor of glycosphingolipid biosynthesis on its own which showed no activity. It is therefore a surprising finding of the present invention that a SERCA antagonist can advantageously be used in combination with an inhibitor of glycosphingolipid biosynthesis in order to treat infections caused by pathogenic mycobacteria.

Infections caused by pathogenic mycobacteria can advantageously therefore be treated by the combination of a SERCA antagonist and an inhibitor of glycosphingolipid biosynthesis.

An infection by a pathogenic mycobacterium, which is treated in accordance with the present invention, is an infection caused by one or more pathogenic Mycobacteria. In other words, it is a pathogenic mycobacterial infection. Such infections include the infectious disease tuberculosis.

Accordingly, in one embodiment of the invention, the infection by a pathogenic mycobacterium is tuberculosis.

The pathogenic mycobacterium which most commonly causes tuberculosis in humans is Mycobacterium tuberculosis. However, other pathogenic mycobacteria, such as Mycobacterium bovis, Mycobacterium africanum, Mycobacterium cannetti, and Mycobacterium microtti, also cause tuberculosis (although these species are less common in humans).

Thus, in one embodiment, the infection by a pathogenic mycobacterium which is treated in accordance with the present invention is an infection caused by Mycobacterium tuberculosis, Mycobacterium bovis, Mycobacterium africanum, Mycobacterium cannetti or Mycobacterium microtti.

In a preferred embodiment, the infection by a pathogenic mycobacterium which is treated in accordance with the present invention is an infection caused by Mycobacterium tuberculosis.

In one preferred embodiment, the infection by a pathogenic mycobacterium which is treated in accordance with the present invention is an infectious disease. Typically, the infectious disease is tuberculosis.

Thus, the present invention provides a combination comprising a SERCA antagonist and an inhibitor of glycosphingolipid biosynthesis, which combination is for use in the treatment of an infection caused by Mycobacterium tuberculosis.

The invention also provides a pharmaceutical composition comprising a SERCA antagonist, an inhibitor of glycosphingolipid biosynthesis, and a pharmaceutically acceptable carrier or diluent, which composition is for use in the treatment of an infection caused by Mycobacterium tuberculosis.

The invention also provides a SERCA antagonist, which SERCA antagonist is for use in the treatment of an infection caused by Mycobacterium tuberculosis, by simultaneous, concurrent, separate or sequential co-administration with an inhibitor of glycosphingolipid biosynthesis.

The invention also provides an inhibitor of glycosphingolipid biosynthesis, which inhibitor of glycosphingolipid biosynthesis is for use in the treatment of an infection caused by Mycobacterium tuberculosis, by simultaneous, concurrent, separate or sequential co-administration with a SERCA antagonist.

The invention also provides a method of treatment of an infection caused by Mycobacterium tuberculosis, which method comprises administering to a human or animal patient in need of such treatment an effective amount of a SERCA antagonist and an effective amount of an inhibitor of glycosphingolipid biosynthesis.

The invention also provides a kit of parts comprising a SERCA antagonist together with instructions for simultaneous, concurrent, separate or sequential use in combination with an inhibitor of glycosphingolipid biosynthesis, for the treatment of a human or animal patient suffering from or susceptible to an infection caused by Mycobacterium tuberculosis.

The invention also provides a kit of parts comprising an inhibitor of glycosphingolipid biosynthesis together with instructions for simultaneous, concurrent, separate or sequential use in combination with a SERCA antagonist, for the treatment of a human or animal patient suffering from or susceptible to an infection caused by Mycobacterium tuberculosis.

The invention also provides a kit of parts comprising an inhibitor of glycosphingolipid biosynthesis, a SERCA antagonist, and instructions for their simultaneous, concurrent, separate or sequential use for the treatment of a human or animal patient suffering from or susceptible to an infection caused by Mycobacterium tuberculosis.

Preferably said patient is human.

### Preferred Combinations of Agents

Typically, the SERCA antagonist employed in the present invention is selected from: curcumin, thapsigargin, nortrilobolide and cyclopiazonic acid, and pharmaceutically acceptable salts thereof; and the inhibitor of glycosphingolipid biosynthesis employed in the present invention is selected from an iminosugar, D,L-threo-1-phenyl-2-decanoylamino-3-morpholino-1-propanol (PDMP); D,L-threo-1-phenyl-2-hexadecanoylamino-3-morpholino-1-propanol (PPMP); D-threo-1-phenyl-2-palmitoilamino-3-pyrrolidino-1-propanol (P4); 4′-hydroxy-D-threo-1-phenyl-2-palmitoilamino-3-pyrrolidino-1-propanol (4′-hydroxy-P4); 3′,4′-ethylenedioxy-P4 (EtDO-P4); N-[(1R,2R)-1-(2,3-Dihydro-1,4-benzodioxin-6-yl)-1-hydroxy-3-(1-pyrrolidinyl)-2-propanyl]octanamide (eliglustat); and 2,5-dihydroxymethyl-3,4-dihydroxypyrrolidine (L-DMDP), and pharmaceutically acceptable salts thereof.

For instance, the SERCA antagonist employed in the present invention may be selected from: curcumin, thapsigargin, nortrilobolide and cyclopiazonic acid, and pharmaceutically acceptable salts thereof; and the inhibitor of glycosphingolipid biosynthesis employed in the present invention may be selected from an iminosugar, N-[(1R,2R)-1-(2,3-Dihydro-1,4-benzodioxin-6-yl)-1-hydroxy-3-(1-pyrrolidinyl)-2-propanyl]octanamide (eliglustat), and pharmaceutically acceptable salts thereof. An example of a pharmaceutically acceptable salt of eliglustat which may be employed is eliglustat tartrate.

More preferably, the SERCA antagonist is selected from curcumin, thapsigargin and cyclopiazonic acid, and pharmaceutically acceptable salts thereof; and the inhibitor of glycosphingolipid biosynthesis is an iminosugar or a pharmaceutically acceptable salt of an iminosugar.

In an alternative preferable embodiment, the SERCA antagonist is selected from curcumin, thapsigargin and cyclopiazonic acid, and pharmaceutically acceptable salts thereof; and the inhibitor of glycosphingolipid biosynthesis is N-[(1R,2R)-1-(2,3-Dihydro-1,4-benzodioxin-6-yl)-1-hydroxy-3-(1-pyrrolidinyl)-2-propanyl] octanamide (eliglustat) or a pharmaceutically acceptable salt of eliglustat. An example of a preferred pharmaceutically acceptable salt of eliglustat is eliglustat tartrate.

In an even more preferred alternative embodiment, the SERCA antagonist is curcumin or a pharmaceutically acceptable salt thereof; and the inhibitor of glycosphingolipid biosynthesis is N-[(1R,2R)-1-(2,3-Dihydro-1,4-benzodioxin-6-yl)-1-hydroxy-3-(1-pyrrolidinyl)-2-propanyl]octanamide (eliglustat) or a pharmaceutically acceptable salt of eliglustat. An example of a preferred pharmaceutically acceptable salt of eliglustat is eliglustat tartrate.

Even more preferably, the SERCA antagonist is curcumin or a pharmaceutically acceptable salt thereof; and the inhibitor of glycosphingolipid biosynthesis is an iminosugar or a pharmaceutically acceptable salt of an iminosugar.

In a particularly preferred embodiment of the present invention, the SERCA antagonist is curcumin or a pharmaceutically acceptable salt thereof; and the inhibitor of glycosphingolipid biosynthesis is N-butyldeoxynojirimycin (NB-DNJ), N-butyldeoxygalactonojirimycin (NB-DGJ) or a pharmaceutically acceptable salt thereof.

In the most preferred embodiment of the invention, the SERCA antagonist is curcumin or a pharmaceutically acceptable salt thereof; and the inhibitor of glycosphingolipid biosynthesis is N-butyldeoxynojirimycin (NB-DNJ), or a pharmaceutically acceptable salt thereof.

When the inhibitor of glycosphingolipid biosynthesis employed in the present invention is an iminosugar, the iminosugar is typically a compound of formula (Ia) or a pharmaceutically acceptable salt thereof

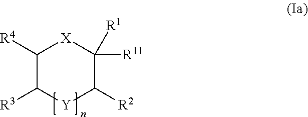

wherein X is NR⁵; n is 1; Y is CHR⁶; R¹¹ is H;

R¹ and R⁴, which may be the same or different, are independently selected from hydrogen, hydroxyl, carboxyl, amino, thiol, substituted or unsubstituted C₁₋₂₀ alkyl, substituted or unsubstituted C₁₋₂₀ alkoxy, substituted or unsubstituted C₁₋₂₀ alkylene-C₃₋₂₅ cycloalkyl, substituted or unsubstituted aryloxy, acyl, ester, acyloxy, C₁₋₁₀ alkylamino, di(C₁₋₁₀)alkylamino, amido, acylamido, —O—C₃₋₂₅ cycloalkyl and —O—C₃₋₂₀ heterocyclyl, provided that one of R¹ and R⁴ may form, together with R⁵, a substituted or unsubstituted C₁₋₆ alkylene group, wherein said C₁₋₂₀ alkyl and C₁₋₂₀ alkylene are optionally interrupted by N(R′), O, S or arylene;

R² and R³, which may be the same or different, are independently selected from hydrogen, hydroxyl, carboxyl, amino, thiol, substituted or unsubstituted C₁₋₂₀ alkyl, substituted or unsubstituted C₁₋₂₀ alkoxy, substituted or unsubstituted —O—C₁₋₂₀ alkylene-C₃₋₂₅ cycloalkyl, substituted or unsubstituted aryloxy, acyl, ester, acyloxy, C₁₋₁₀alkylamino, di(C₁₋₁₀)alkylamino, amido, acylamido, —O—C₃₋₂₅ cycloalkyl and —O—C₃₋₂₀ heterocyclyl, wherein said C₁₋₂₀ alkyl and C₁₋₂₀ alkylene are optionally interrupted by N(R′), O, S or arylene; and

R⁵ is hydrogen, substituted or unsubstituted C₁₋₂₀ alkyl, substituted or unsubstituted C₁₋₂₀ alkylene-aryl, substituted or unsubstituted C₁₋₂₀ alkylene-C₃₋₂₀ heteroaryl, substituted or unsubstituted C₁₋₂₀ alkylene-C₃₋₂₅ cycloalkyl, substituted or unsubstituted C₁₋₂₀ alkylene-C₃₋₂₀ heterocyclyl, substituted or unsubstituted C₁₋₂₀ alkylene-O—C₃₋₂₀ heterocyclyl or R⁵ forms, together with R¹ or R⁴, a substituted or unsubstituted C₁₋₆ alkylene group, wherein said C₁₋₂₀ alkyl and C₁₋₂₀ alkylene are optionally interrupted by N(R′), O, S or arylene wherein R′ is H, C₁₋₆ alkyl or aryl; and

R⁶ is selected from hydrogen, hydroxyl, acyloxy, amino, substituted or unsubstituted C₁₋₂₀ alkoxy and substituted or unsubstituted C₁₋₂₀ alkyl.

Typically, when R¹, R⁴ or R⁵ is substituted or unsubstituted C₁₋₂₀ alkylene-C₃₋₂₅ cycloalkyl, the C₁₋₂₀ alkylene is interrupted once by O and the C₃₋₂₅ cycloalkyl is Adamantyl, and thus the R¹, R⁴ or R⁵ is —(C₁₋₁₀ alkylene)-O—CH₂-Adamantyl. This includes, for instance, —(CH₂)₅—O—CH₂-Adamantyl.

Similarly, when R² or R³ is substituted or unsubstituted —O—C₁₋₂₀ alkylene-C₃₋₂₅ cycloalkyl, the C₁₋₂₀ alkylene is often interrupted once by O and the C₃₋₂₅ cycloalkyl is Adamantyl, and thus the R² or R³ is —O—(C₁-10 alkylene)-O—CH₂-Adamantyl. This includes, for instance, —O—(CH₂)₅—O—CH₂-Adamantyl.

The iminosugar may for instance be selected from:

N-butyldeoxynojirimycin (NB-DNJ), also known as miglustat or ZAVESCA®; N-nonyldeoxynojirimycin (NN-DNJ); N-butyldeoxygalactonojirimycin (NB-DGJ); N-5-adamantane-1-yl-methoxypentyl-deoxynojirimycin (AMP-DNJ); alpha-homogalactonojirimycin (HGJ); Nojirimycin (NJ); Deoxynojirimycin (DNJ); N7-oxadecyl-deoxynojirimycin; deoxygalactonojirimycin (DGJ); N-butyl-deoxygalactonojirimycin (NB-DGJ); N-nonyl-deoxygalactonojirimycin (NN-DGJ); N-nonyl-6deoxygalactonojirimycin; N7-oxanonyl-6deoxy-DGJ; alpha-homoallonojirimycin (HAJ); beta-1-C-butyl-deoxygalactonojirimycin (CB-DGJ); Castanospermine; MDL25874; 1,5-dideoxy-1,5-imino-D-glucitol, and their N-alkyl, N-acyl and N-aryl, and optionally O-acylated derivatives; 1,5-(Methylimino)-1,5-dideoxy-D-glucitol; 1,5-(Hexylimino)-1,5-dideoxy-D-glucitol; 1,5-(Nonylylimino)-1,5-dideoxy-D-glucitol; 1,5-(2-Ethylbutylimino)-1,5-dideoxy-D-glucitol; 1,5-(2-Methylpentylimino)-1,5-dideoxy-D-glucitol; 1,5-(Benzyloxycarbonylimino)-1,5-dideoxy-D-glucitol, tetraacetate; 1,5-(Phenylacetylimino)-1,5-dideoxy-D-glucitol, tetraacetate; 1,5-(Benzoylimino)-1,5-dideoxy-D-glucitol, tetraacetate; 1,5-(Butylimino)-1,5-dideoxy-D-glucitol, tetraacetate; 1,5-(Ethyl malonylimino)-1,5-dideoxy-D-glucitol, tetraacetate; 1,5-(Hexylimino)-1,5-dideoxy-D-glucitol, tetraacetate; 1,5-(Nonylimino)-1,5-dideoxy-D-glucitol, tetraacetate; 1,5-(Benzyloxycarbonylimino)-1,5-dideoxy-D-glucitol, tetraisobutyrate; 1,5-(Butylimino)-1,5-dideoxy-D-glucitol, tetrabutyrate; 1,5-(Butylimino)-1,5-dideoxy-D-glucitol, tetrapropionate; 1,5-(Butylimino)-1,5-dideoxy-D-glucitol, tetrabenzoate; 1,5-Dideoxy-1,5-imino-D-glucitol, tetraisobutyrate; 1,5-(Hydrocinnamoylimino)-1,5-dideoxy-D-glucitol, tetraacetate; 1,5-(Methyl malonylimino)-1,5-dideoxy-D-glucitol, tetraacetate; 1,5-(Butylimino)-1,5-dideoxy-D-glucitol, tetraisobutyrate; 1,5-(Butylimino)-1,5-dideoxy-4R,6—O-(phenylmethylene)-D-glucitol, diacetate; 1,5-[(Phenoxymethyl)carbonylimino]-1,5-dideoxy-D-glucitol, tetraacetate; 1,5-[(Ethylbutyl)imino]-1,5-dideoxy-D-glucitol, tetraacetate; 1,5-(Butylimino)-1,5-dideoxy-D-glucitol, 2,3-diacetate; 1,5-(Hexylimino)-1,5-dideoxy-4R,6-O-(phenylmethylene)-D-glucitol, diacetate; 1,5-(Hexylimino)-1,5-dideoxy-D-glucitol, 2,3-diacetate; 1,5-[(2-Methylpentyl)imino]-1,5-dideoxy-D-glucitol, tetraacetate; 1,5-(Butylimino)-1,5-dideoxy-D-glucitol, 6-acetate; 1,5-[(3-Nicotinoyl)imino]-1,5-dideoxy-D-glucitol, tetraacetate; 1,5-(Cinnamoylimino)-1,5-dideoxy-D-glucitol, tetraacetate; 1,5-(Butylimino)-1,5-dideoxy-D-glucitol, 2,3-dibutyrate; 1,5-(Butylimino)-1,5-dideoxy-4R,6-O-(phenylmethylene)-D-glucitol, 2,3-dibutyrate; 1,5-(Phenylacetylimino)-1,5-dideoxy-D-glucitol, tetraisobutyrate; 1,5-[(4-Chlorophenyl)acetylimino]-1,5-dideoxy-D-glucitol, tetraacetate; 1,5-[(4-Biphenyl)acetylimino]-1,5-dideoxy-D-glucitol, tetraacetate; 1,5-(Benzyloxycarbonylimino)-1,5-dideoxy-D-glucitol, tetrabutyrate; 1,5-Dideoxy-1,5-imino-D-glucitol, tetrabutyrate; 3,4,5-piperidinetriol, 1-propyl-2-(hydroxymethyl)-, (2S, 3R, 4R, 5S); 3,4,5-piperidinetriol, 1-pentyl-2-(hydroxymethyl)-, (2S, 3R, 4R, 5S); 3,4,5-piperidinetriol, 1-heptyl-2-(hydroxymethyl)-, (2S, 3R, 4R, 5S); 3,4,5-piperidinetriol, 1-butyl-2-(hydroxymethyl)-, (2S, 3S, 4R, 5S); 3,4,5-piperidinetriol, 1-nonyl-2-(hydroxymethyl)-, (2S, 3R, 4R, 5S); 3,4,5-piperidinetriol, 1-(1-ethyl) propyl-2-(hydroxymethyl)-, (2S, 3R, 4R, 5S); 3,4,5-piperidinetriol, 1-(3-methyl) butyl-2-(hydroxymethyl)-, (2S, 3R, 4R, 5S); 3,4,5-piperidinetriol, 1-(2-phenyl) ethyl-2-(hydroxymethyl)-, (2S, 3R, 4R, 5S); 3,4,5-piperidinetriol, 1-(3-phenyl) propyl-2-(hydroxymethyl)-, (2S, 3R, 4R, 5S); 3,4,5-piperidinetriol, 1-(1-ethyl) hexyl-2-(hydroxymethyl)-, (2S, 3R, 4R, 5S); 3,4,5-piperidinetriol, 1-(2-ethyl) butyl-2-(hydroxymethyl)-, (2S, 3R, 4R, 5S); 3,4,5-piperidinetriol, 1-[(2R)-(2-methyl-2-phenyl) ethyl]-2-(hydroxymethyl)-, (2S, 3R, 4R, 5S); 3,4,5-piperidinetriol, 1-[(2S)-(2-methyl-2-phenyl) ethyl]-2-(hydroxymethyl)-, (2S, 3R, 4R, 5S), β-L-homofuconojirimycin; and propyl 2-acetamido-2-deoxy-4-O-(fl-D-galactopyranosyl)-3-O-(2-(N-(fl-L-homofuconojirimycinyl))ethyl)-α-D-glucopyranoside; ido-N-(5-adamantane-1-yl-methoxy-pentyl)deoxynojirimycin; N-(adamantane-1-yl-methoxypentyl)-L-ido-deoxynojirimycin; N-(adamantane-1-yl-methoxypentyl)-D-galacto-deoxynojirimycin; C1-beta-(adamantane-1-yl-methoxypentyl)-deoxynojirimycin; N-methyl-C1-beta-(adamantane-1-yl-methoxypentyl)-deoxynojirimycin; N-butyl-C1-beta-(adamantane-1-yl-methoxypentyl)-deoxynojirimycin; 2-O-(adamantane-1-yl-methoxypentyl)-deoxynojirimycin; N-methyl-2-O-(adamantane-1-yl-methoxy-pentyl)-deoxynojirimycin; N-butyl-2-O-(adamantane-1-yl-methoxy-pentyl)-deoxynojirimycin; N-benzyloxycarbonyl-2-O-(adamantane-1-yl-methoxypentyl)-3,4,6-tri-O-benzyl-deoxy-nojirimycin; and N-(5-adamantane-1-yl-methoxy-pentyl)deoxynojirimycin

The iminosugar is preferably however selected from N-butyldeoxynojirimycin (NB-DNJ), N-butyldeoxygalactonojirimycin (NB-DGJ), ido-N-(5-adamantane-1-yl-methoxy-pentyl)deoxynojirimycin; N-(adamantane-1-yl-methoxypentyl)-L-ido-deoxynojirimycin; N-(adamantane-1-yl-methoxypentyl)-D-galacto-deoxynojirimycin; C1-beta-(adamantane-1-yl-methoxypentyl)-deoxynojirimycin; N-methyl-C1-beta-(adamantane-1-yl-methoxypentyl)-deoxynojirimycin; N-butyl-C1-beta-(adamantane-1-yl-methoxypentyl)-deoxynojirimycin; 2-O-(adamantane-1-yl-methoxypentyl)-deoxynojirimycin; N-methyl-2-O-(adamantane-1-yl-methoxy-pentyl)-deoxynojirimycin; N-butyl-2-O-(adamantane-1-yl-methoxy-pentyl)-deoxynojirimycin; N-benzyloxycarbonyl-2-O-(adamantane-1-yl-methoxypentyl)-3,4,6-tri-O-benzyl-deoxy-nojirimycin; and N-(5-adamantane-1-yl-methoxy-pentyl)deoxynojirimycin.

Even more preferably, the iminosugar is N-butyldeoxynojirimycin (NB-DNJ) or N-butyldeoxygalactonojirimycin (NB-DGJ).

Most preferably, the iminosugar is N-butyldeoxynojirimycin (NB-DNJ), which is also known as miglustat and ZAVESCA®.

NB-DGJ may also be preferred, however, due to its advantageous side effect profile. Thus, in another highly preferred embodiment the iminosugar is NB-DGJ.

### Preferred Treatments by Preferred Combinations

In a preferred embodiment, the infection by a pathogenic mycobacterium which is treated in accordance with the present invention is an infection caused by Mycobacterium tuberculosis; the SERCA antagonist employed is selected from: curcumin, thapsigargin, nortrilobolide and cyclopiazonic acid, and pharmaceutically acceptable salts thereof, and the inhibitor of glycosphingolipid biosynthesis employed is selected from an iminosugar, D,L-threo-1-phenyl-2-decanoylamino-3-morpholino-1-propanol (PDMP); D,L-threo-1-phenyl-2-hexadecanoylamino-3-morpholino-1-propanol (PPMP); D-threo-1-phenyl-2-palmitoilamino-3-pyrrolidino-1-propanol (P4); 4′-hydroxy-D-threo-1-phenyl-2-palmitoilamino-3-pyrrolidino-1-propanol (4′-hydroxy-P4); 3′,4′-ethylenedioxy-P4 (EtDO-P4); N-[(1R,2R)-1-(2,3-Dihydro-1,4-benzodioxin-6-yl)-1-hydroxy-3-(1-pyrrolidinyl)-2-propanyl]octanamide (eliglustat); and 2,5-dihydroxymethyl-3,4-dihydroxypyrrolidine (L-DMDP), and pharmaceutically acceptable salts thereof.

In an even more preferred embodiment, the infection by a pathogenic mycobacterium which is treated in accordance with the present invention is an infection caused by Mycobacterium tuberculosis; the SERCA antagonist employed is selected from: curcumin, thapsigargin, nortrilobolide and cyclopiazonic acid, and pharmaceutically acceptable salts thereof; and the inhibitor of glycosphingolipid biosynthesis employed is selected from an iminosugar, N-[(1R,2R)-1-(2,3-Dihydro-1,4-benzodioxin-6-yl)-1-hydroxy-3-(1-pyrrolidinyl)-2-propanyl]octanamide (eliglustat) and pharmaceutically acceptable salts thereof.

For instance, in one preferred embodiment, the infection by a pathogenic mycobacterium which is treated in accordance with the present invention is an infection caused by Mycobacterium tuberculosis; the SERCA antagonist is selected from curcumin, thapsigargin and cyclopiazonic acid, and pharmaceutically acceptable salts thereof; and the inhibitor of glycosphingolipid biosynthesis is an iminosugar or eliglustat or a pharmaceutically acceptable salt of an iminosugar or eliglustat.

Thus, in a preferred embodiment, the infection by a pathogenic mycobacterium which is treated in accordance with the present invention is an infection caused by Mycobacterium tuberculosis; the SERCA antagonist is curcumin or a pharmaceutically acceptable salt thereof; and the inhibitor of glycosphingolipid biosynthesis is an iminosugar or a pharmaceutically acceptable salt of an iminosugar.

In another preferred embodiment, the infection by a pathogenic mycobacterium which is treated in accordance with the present invention is an infection caused by Mycobacterium tuberculosis; the SERCA antagonist is curcumin or a pharmaceutically acceptable salt thereof; and the inhibitor of glycosphingolipid biosynthesis is eliglustat or a pharmaceutically acceptable salt of eliglustat. The pharmaceutically acceptable salt of eliglustat may be eliglustat tartrate.

In a particularly preferred embodiment, the infection by a pathogenic mycobacterium which is treated in accordance with the present invention is an infection caused by Mycobacterium tuberculosis; the SERCA antagonist is selected from curcumin, thapsigargin and cyclopiazonic acid, and pharmaceutically acceptable salts thereof; and the inhibitor of glycosphingolipid biosynthesis is an iminosugar or a pharmaceutically acceptable salt of an iminosugar.

In an even more preferred embodiment, the infection by a pathogenic mycobacterium which is treated in accordance with the present invention is an infection caused by Mycobacterium tuberculosis; the SERCA antagonist is curcumin or a pharmaceutically acceptable salt thereof; and the inhibitor of glycosphingolipid biosynthesis is N-butyldeoxynojirimycin (NB-DNJ), N-butyldeoxygalactonojirimycin (NB-DGJ) or a pharmaceutically acceptable salt thereof.

Thus, in one more preferred embodiment, the infection by a pathogenic mycobacterium which is treated in accordance with the present invention is an infection caused by Mycobacterium tuberculosis; the SERCA antagonist is curcumin or a pharmaceutically acceptable salt thereof; and the inhibitor of glycosphingolipid biosynthesis is N-butyldeoxygalactonojirimycin (NB-DGJ) or a pharmaceutically acceptable salt thereof.

In the most preferred embodiment, the infection by a pathogenic mycobacterium which is treated in accordance with the present invention is an infection caused by Mycobacterium tuberculosis; the SERCA antagonist is curcumin or a pharmaceutically acceptable salt thereof; and the inhibitor of glycosphingolipid biosynthesis is N-butyldeoxynojirimycin (NB-DNJ), or a pharmaceutically acceptable salt thereof.

Thus, the present invention provides a combination comprising curcumin or a pharmaceutically acceptable salt thereof and N-butyldeoxynojirimycin (NB-DNJ) or a pharmaceutically acceptable salt thereof, which combination is for use in the treatment of an infection caused by Mycobacterium tuberculosis.

The invention also provides a pharmaceutical composition comprising curcumin or a pharmaceutically acceptable salt thereof, N-butyldeoxynojirimycin (NB-DNJ) or a pharmaceutically acceptable salt thereof, and a pharmaceutically acceptable carrier or diluent, which composition is for use in the treatment of an infection caused by Mycobacterium tuberculosis.

The invention also provides curcumin or a pharmaceutically acceptable salt thereof, for use in the treatment of an infection caused by Mycobacterium tuberculosis, by simultaneous, concurrent, separate or sequential co-administration with N-butyldeoxynojirimycin (NB-DNJ) or a pharmaceutically acceptable salt thereof.

The invention also provides N-butyldeoxynojirimycin (NB-DNJ) or a pharmaceutically acceptable salt thereof, for use in the treatment of an infection caused by Mycobacterium tuberculosis, by simultaneous, concurrent, separate or sequential co-administration with curcumin or a pharmaceutically acceptable salt thereof.

The invention also provides a method of treatment of an infection caused by Mycobacterium tuberculosis, which method comprises administering to a human or animal patient in need of such treatment an effective amount of curcumin or a pharmaceutically acceptable salt thereof and an effective amount of N-butyldeoxynojirimycin (NB-DNJ) or a pharmaceutically acceptable salt thereof.

Preferably said patient is human.

The invention also provides a kit of parts comprising curcumin or a pharmaceutically acceptable salt thereof together with instructions for simultaneous, concurrent, separate or sequential use in combination with N-butyldeoxynojirimycin (NB-DNJ) or a pharmaceutically acceptable salt thereof, for the treatment of a human or animal patient suffering from or susceptible to an infection caused by Mycobacterium tuberculosis.

Preferably said patient is human.

The invention also provides a kit of parts comprising N-butyldeoxynojirimycin (NB-DNJ) or a pharmaceutically acceptable salt thereof together with instructions for simultaneous, concurrent, separate or sequential use in combination with curcumin or a pharmaceutically acceptable salt thereof, for the treatment of a human or animal patient suffering from or susceptible to an infection caused by Mycobacterium tuberculosis.

Preferably said patient is human.

The invention also provides a kit of parts comprising N-butyldeoxynojirimycin (NB-DNJ) or a pharmaceutically acceptable salt thereof, curcumin or a pharmaceutically acceptable salt thereof, and instructions for their simultaneous, concurrent, separate or sequential use for the treatment of a human or animal patient suffering from or susceptible to an infection caused by Mycobacterium tuberculosis.

Preferably said patient is human.

### Administration Details

The active compounds employed in the method of the invention, i.e. the SERCA antagonist and the inhibitor of glycosphingolipid biosynthesis, may be administered together in the same pharmaceutical composition. Alternatively, they may be administered in different compositions, and the different compositions may be administered separately, simultaneously, concomitantly or sequentially, and by the same route or by a different route.

Whether they are administered together in the same composition, or in different compositions, the SERCA antagonist and the inhibitor of glycosphingolipid biosynthesis can be administered in a variety of dosage forms, for example orally such as in the form of tablets, capsules, sugar- or film-coated tablets, liquid solutions or suspensions or parenterally, for example intramuscularly, intravenously or subcutaneously. The compounds may therefore be given by injection or infusion.

Whether they are administered together in the same composition, or in different compositions, the SERCA antagonist and the inhibitor of glycosphingolipid biosynthesis may be presented for administration in a liposome. Thus, either or both compounds may be encapsulated or entrapped in a liposome and then administered to the patient to be treated. Active ingredients encapsulated by liposomes may reduce toxicity, increase efficacy, or both. Notably, liposomes are thought to interact with cells by stable absorption, endocytosis, lipid transfer, and fusion (R. B. Egerdie et al., 1989, J. Urol. 142:390).

The dosages of the SERCA antagonist and the inhibitor of glycosphingolipid biosynthesis may be the same or different from one another and depend on a variety of factors including the age, weight and condition of the patient and the route of administration. Daily dosages can vary within wide limits and will be adjusted to the individual requirements in each particular case. Typically, however, the dosage of the SERCA antagonist, adopted for each route of administration, when the compound is co-administered with the inhibitor of glycosphingolipid biosynthesis to adult humans, is 0.0001 to 50 mg/kg, most commonly in the range of 0.001 to 10 mg/kg, body weight, for instance 0.01 to 1 mg/kg. Such a dosage may be given, for example, from 1 to 5 times daily. For intravenous injection a suitable daily dose is from 0.0001 to 1 mg/kg body weight, preferably from 0.0001 to 0.1 mg/kg body weight. A daily dosage can be administered as a single dosage or according to a divided dose schedule. Similarly, the dosage of the inhibitor of glycosphingolipid biosynthesis, adopted for each route of administration, when the compound is co-administered with the SERCA antagonist to adult humans, is typically 0.0001 to 50 mg/kg, most commonly in the range of 0.001 to 10 mg/kg, body weight, for instance 0.01 to 1 mg/kg. Such a dosage may be given, for example, from 1 to 5 times daily. For intravenous injection a suitable daily dose is from 0.0001 to 1 mg/kg body weight, preferably from 0.0001 to 0.1 mg/kg body weight. A daily dosage can be administered as a single dosage or according to a divided dose schedule.

Typically the doses of each active agent (i.e. the dose of the SERCA antagonist and the dose of the inhibitor of glycosphingolipid biosynthesis), which may be the same or different, to treat human patients range from about 0.1 mg to about 1000 mg of the compound, more typically from about 10 mg to about 1000 mg of the compound. A typical dose may be about 100 mg to about 300 mg of the compound. A dose may be administered once a day (QID), twice per day (BID), or more frequently, depending on the pharmacokinetic and pharmacodynamic properties, including absorption, distribution, metabolism, and excretion of the particular compound. In addition, toxicity factors may influence the dosage and administration regimen. When administered orally, the pill, capsule, or tablet may be ingested daily or less frequently for a specified period of time. The regimen may be repeated for a number of cycles of therapy.

The proportions in which (a) the SERCA antagonist and (b) the inhibitor of glycosphingolipid biosynthesis may be used according to the invention are variable. Active substances (a) and (b) may possibly be present in the form of their solvates or hydrates, or in any of various salt forms. Depending on the choice of the compounds (a) and (b), and the forms of those compounds (e.g. salt form, solvated, hydrated), the weight ratios which may be used within the scope of the present invention vary on the basis of the different molecular weights. The pharmaceutical combinations according to the invention may for instance contain (a) and (b) generally in a ratio by weight (b):(a) ranging from 1:500 to 500:1, preferably from 1:100 to 100:1, for instance from 1:10 to 10:1.

The SERCA antagonist and/or the inhibitor of glycosphingolipid biosynthesis compound may be formulated for use as a pharmaceutical composition also comprising a pharmaceutically acceptable carrier or diluent. Such compositions are typically prepared following conventional methods and are administered in a pharmaceutically suitable form. The SERCA antagonist and the inhibitor of glycosphingolipid biosynthesis, may be administered together in the same pharmaceutical composition. Alternatively, they may be administered in different compositions, and the different compositions may be administered separately, simultaneously, concomitantly or sequentially, and by the same route or by a different route. Thus, either or both compounds may be administered in any conventional form and route, for instance as follows:

A) Orally, for example, as tablets, coated tablets, dragees, troches, lozenges, aqueous or oily suspensions, liquid solutions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of sweetening agents, flavouring agents, colouring agents and preserving agents in order to provide pharmaceutically elegant and palatable preparations.

Tablets contain the active ingredient in admixture with non-toxic pharmaceutically acceptable excipients which are suitable for the manufacture of tablets. These excipients may be for example, inert diluents, such as calcium carbonate, sodium carbonate, lactose, dextrose, saccharose, cellulose, corn starch, potato starch, calcium phosphate or sodium phosphate; granulating and disintegrating agents, for example, maize starch, alginic acid, alginates or sodium starch glycolate; binding agents, for example starch, gelatin or acacia; lubricating agents, for example silica, magnesium or calcium stearate, stearic acid or talc; effervescing mixtures; dyestuffs, sweeteners, wetting agents such as lecithin, polysorbates or lauryl sulphate. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed. Such preparations may be manufactured in a known manner, for example by means of mixing, granulating, tableting, sugar coating or film coating processes.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is present as such, or mixed with water or an oil medium, for example, peanut oil, liquid paraffin, or olive oil.

Aqueous suspensions contain the active materials in admixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides for example polyoxyethylene sorbitan monooleate.

The said aqueous suspensions may also contain one or more preservatives, for example, ethyl or n-propyl p-hydroxybenzoate, one or more colouring agents, such as sucrose or saccharin.

Oily suspension may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavouring agents may be added to provide a palatable oral preparation. These compositions may be preserved by this addition of an antioxidant such as ascorbic acid. Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavouring and colouring agents, may also be present.

Pharmaceutical compositions for use in accordance with the invention may also be in the form of oil-in-water emulsions. The oily phase may be a vegetable oil, for example olive oil or arachis oils, or a mineral oil, for example liquid paraffin or mixtures of these.

Suitable emulsifying agents may be naturally-occurring gums, for example gum acacia or gum tragacanth, naturally occurring phosphatides, for example soy bean lecithin, and esters or partial esters derived from fatty acids an hexitol anhydrides, for example sorbitan monooleate, and condensation products of the said partial esters with ethylene oxide, for example polyoxyethylene sorbitan monooleate. The emulsion may also contain sweetening and flavouring agents. Syrups and elixirs may be formulated with sweetening agents, for example glycerol, sorbitol or sucrose. In particular a syrup for diabetic patients can contain as carriers only products, for example sorbitol, which do not metabolise to glucose or which only metabolise a very small amount to glucose.

Such formulations may also contain a demulcent, a preservative and flavouring and coloring agents;

B) Parenterally, either subcutaneously, or intravenously, or intramuscularly, or intrastemally, or by infusion techniques, in the form of sterile injectable aqueous or oleaginous suspensions. This suspension may be formulated according to the known art using those suitable dispersing of wetting agents and suspending agents which have been mentioned above. The sterile injectable preparation may also be a sterile injectable solution or suspension in a non-toxic paternally-acceptable diluent or solvent, for example as a solution in 1,3-butane diol.

Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition fatty acids such as oleic acid find use in the preparation of injectables;

C) By inhalation, in the form of aerosols or solutions for nebulizers;

D) Rectally, in the form of suppositories prepared by mixing the drug with a suitable non-irritating excipient which is solid at ordinary temperature but liquid at the rectal temperature and will therefore melt in the rectum to release the drug. Such materials are cocoa butter and poly-ethylene glycols;

E) Topically, in the form of creams, ointments, jellies, collyriums, solutions or suspensions.

F) Vaginally, in the form of pessaries, tampons, creams, gels, pastes, foams or spray formulations containing in addition to the active ingredient such carriers as are known in the art to be appropriate.

The present invention is further illustrated in the Examples which follow:

### EXAMPLES

### Example 1

Methods

Cells.

RAW 264.7 macrophages were obtained from the European Cell Culture Collection (Porton Down, UK). Primary mouse macrophages were isolated from adult (8 week old) mice and cultured at 37° C. with 5% CO₂ in RPMI with 10% foetal calf serum (FCS), 1% penicillin/streptomycin and 1% L-glutamine (Lonza, Basel, Switzerland). Mtb (H37Rv strain) and M. bovis BCG (Pasteur strain) were kindly provided by Simon Clark and Dominic Kelly. Fluorescent M. smegmatis (mc²155 strain expressing mCherry) was provided by David Russell. Mycobacteria were grown on 7H11 agar plates (supplemented with Oleic Albumin Dextrose Catalase) before transfer to 7H9 liquid media (supplemented with Albumin Dextrose Catalase). NPC1-overexpressing Chinese Hamster Ovary (CHO) cells (Millard, E. E., et al. J Biol Chem 275, 38445-38451, doi:10.1074/jbc.M003180200 M003180200 [pii], 2000) were grown at 37° C. with 5% CO₂ in DMEM-F12 with 10% FCS, 1% penicillin/streptomycin and 1% glutamine. U18666A was used at a concentration of 1 μg/ml for 48 hours.

Generation of Human Monocyte-Derived Macrophages.

Peripheral blood CD14⁺ monocytes were isolated using microbeads (Miltenyi Biotec) according to manufacturer's instructions, differentiated in the presence of M-CSF (10 ng/ml) in X-vivo media (Lonza) and used after 7 days.

FLUOS Labelling of Mycobacteria.

A volume (5 ml) of a mid-exponential (OD₆₀₀ 0.8-1.2) mycobacteria culture was centrifuged, resuspended in 500 μl of HEPES buffer (pH 9.1) and labelled with 25 μl of 20 mg/ml FLUOS (5(6)-carboxyfluorescein-N-hydroxysuccinimideester) in DMSO. Bacteria were washed twice with warm 7H9 (37° C.) and resuspended in 500 μl of RPMI-FCS. The OD₆₀₀ of the solution was measured and the concentration of bacteria determined.

Generation of mCherry-Expressing BCG.

BCG was electroporated with pV116 plasmid containing gene for mCherry using standard parameters. Transformed colonies were selected on 7H11 OADC agar plates. Individual colonies were picked and grown in liquid culture.

Host Cell Infection.

Unless otherwise stated infections were carried out at a multiplicity of infection (MOI) of 12.5. Host cells were plated out approximately 18h prior to infection. A volume of mid-exponential mycobacteria was centrifuged and resuspended in appropriate medium prior to dilution.

Purification of Mycobacterial Cell Wall Components.

Total lipids and mycolic acid methyl-esters (MAMES) were extracted from M. bovis. A 100 ml culture of bacteria was grown to an absorbance of 1.0 at OD₆₀₀, centrifuged and the bacteria were resuspended in 5 ml PBS. This was transferred to an 8.5 ml screw top glass culture tube and centrifuged prior to removal of supernatant and drying of pellet overnight at room temperature. The desiccated bacterial pellet was incubated with 2 ml of 5% aqueous tetrabutylammonium hydroxide at 100° C. for 16h. The sample was cooled and 100 μl of methyl iodide, 4 ml dichloromethane and 2 ml H₂O added. The sample was mixed for 30 min and the lower organic layer removed, washed with 3×5 ml H₂O and dried under nitrogen. The dried extract was re-suspended in 1 ml diethyl ether, mixed for 60 min and centrifuged at 3,000×g for 5 min. The supernatant was removed, pellet dried under nitrogen and then resuspended in 500 μl of dichloromethane to give the mycolic acid methyl esters (MAMES) and fatty acid methyl esters (FAMES). The sample was applied to a TLC plate and separated in one dimension with petroleum ether: acetone (95:5) solvent system. The TLC plate was sprayed with 5% (v/v) molybdophosphoric acid and charred at 110° C. to analyze the lipid species. Purified mycolic acid fraction (from Mtb) and trehalose dimycolate (TDM) (from M. bovis) were purchased from Sigma-Aldrich.

Purification of Mycobacterial Lipids

Lipid Extraction and Analysis.

Crude non-polar lipids and polar lipids were initially extracted from Mtb according to published methods by stirring in petroleum ether and methanolic saline for 2h (Tatituri, R. V. et al. J Biol Chem 282, 4561-4572, doi:10.1074/jbc.M608695200, 2007). The biomass was allowed to settle overnight and centrifuged at 3000 rpm for 5 min. The resulting bi-phasic solution was separated and the upper layer containing non-polar lipids recovered. The lower layer was treated with petroleum ether, mixed and harvested as described above. The two upper petroleum ether fractions were combined and dried under reduced pressure. To extract polar lipids, a mixture of CHCl₃/CH₃OH/NaCl solution was added to the lower methanolic saline layer and the solution stirred for 4h and left to settle overnight. This mixture was filtered and the filter cake re-extracted twice with CHCl₃/CH₃OH/NaCl solution. Appropriate amounts of CHCl₃ and NaCl solution were added to the combined filtrates and the mixture stirred for 1h and allowed to settle. The lower layer containing the polar lipids was recovered and dried under reduced pressure. The non-polar and polar lipid extracts were examined by 1D thin-layer chromatography on aluminum TLC plates of silica gel 60 F254 (Merck EMD Millipore, Catalog no. 5554-7). Lipids were visualized by spraying plates either with 5% ethanolic molybdophosphoric acid and charring, α-naphthol/sulfuric acid followed by gentle charring of plates for glycolipids, a Dittmer and Lester reagent which is specific for phospholipids and glycophospholipids or using ninhydrin, a amino specific reagent for detecting amino residues on extracted lipids (Tatituri, R. V. et al. J Biol Chem 282, 4561-4572, doi:10.1074/jbc.M608695200, 2007; Dobson, G. et al. in Chemical Methods in Bacterial Systematics (eds M. Goodfellow & D. E. Minnikin) 237-265 (Academic Press, 1985)).

Purification of Lipid Extract.

After analysing the lipid profiles by TLC, purifications by diethylaminoethyl (DEAE) cellulose chromatography were performed. The crude polar lipid extract was dissolved in Solution A (CHCl₃/CH₃OH (2:1, v/v)) and a few drops of H₂O added as necessary to dissolve the lipids. The polar lipid fraction was eluted using Solution A to remove all mycolates, their glycosylated forms and other zwitterionic lipids. Charged lipids were then eluted using ammonium acetate dissolved in Solution A in a stepwise gradient of increasing concentration of ammonium acetate in chloroform/methanol ranging from 1 mM to 300 mM.

Further Purification of Mycolates.

The glycolate mycolate fraction was further purified by either using silica gel packed into glass columns or by preparative TLC. In the silica gel procedure, mycolate fraction was dissolved in 100% CHCl₃ and initially eluted with CHCl₃/CH₃OH (80:1, v/v) and further eluted with decreasing concentration of CHCl₃ but keeping the CH₃OH constant. The glycomycolate fractions were monitored by TLC on a 10 cm×10 cm aluminum backed TLC plates of silica gel 60 F254 (Merck EMD Millipore,) and developing the plate either in CHCl₃/CH₃OH (80:10, v/v) or CHCl₃/CH₃OH/H₂O (65:25:4 v/v/v). The glycomycolates were visualized by spraying with α-naphthol/sulfuric acid followed by gentle charring. In preparative 1D TLC, mycolate extract was loaded on a 10 cm×20 cm plastic-backed TLC plates of silica gel 60 F254 (Merck EMD Millipore,) run in TLC solvent system CHCl₃/(CH₃)₂CO/CH₃OH/H₂O (50:60:2.5:3 v/v/v/v)/. TLC plates were subsequently sprayed with either ethanolic Rhodamine 6G for detection of non-polar lipids or 1,6-diphenyl-1,3,5-hexatriene for polar lipids. The lipid bands were visualized, marked under UV light and the corresponding purified lipid spots were scraped from the plates, silica extracted and used for biological testing.

Lysosomal pH Measurements.

Lysosomal pH was measured as described (Bach, G., Chen, C. S. & Pagano, R. E. Clin Chim Acta 280, 173-179, 1999) with minor modification. Fluorescein-dextran and Rhodamine-dextran (Sigma) were loaded at 0.25 mg/ml for 12h into cells at 37° C. followed by 12h chase at 37° C. to label lysosomes. Image analysis and quantification of fluorescence was performed using SimplePCI software.

Direct Calcium Quantification.

For documenting Ca²⁺ concentration low affinity Rhod-dextran in conjunction with the Ca²⁺ insensitive Alexa-Fluor 488 dextran (Molecular Probes/Invitrogen, Paisley, UK) were used at concentrations of 0.25 mg/ml and 0.1 mg/ml respectively. Intracellular Ca²⁺ was measured using the membrane permeable Ca²⁺ indicators Fura 2-AM alone or Calcium Green 1-AM in conjunction with Fura Red-AM (all at 5 [symbol] M, Molecular Probes). Cells were loaded as described (Lloyd-Evans, E. et al. Nat Med 14, 1247-1255, doi:nm.1876 [pii] 10.1038/nm. 1876, 2008) for 1 h at room temperature prior to washing and visualization of Ca²⁺ release on a Zeiss LSM 510 confocal (for adherent cells) or cuvette based recording on a Jasco FP-777 (with Ca²⁺ recording module CA-261) for non-adherent cells. Movies were analysed using Magipix and Magigraph software.

Indirect Calcium Quantification.

Cells were loaded with 2 μM Fura 2-AM (Teflabs) in the presence of 0.03% Pluronic F127 (Invitrogen) in a buffer containing (mM: 121 NaCl, 5.4 KCl, 0.8 MgCl2, 1.8 CaCl2, 6 NaHCO₃, 25 HEPES, 10 Glucose) for 45 min at room temperature, followed by a 15 min de-esterification. Cells were washed once with Ca²⁺-free buffer supplemented with 1 mM EGTA and twice with Ca²⁺-free buffer containing 100 mM EGTA and subsequent experiments conducted in this same buffer. Cells were mounted on the stage of an Olympus IX71 microscope equipped with a 40 UApo/340 objective (1.35 NA) and a 12-bit Photometrics Coolsnap HQ2 CCD camera. Cells were excited alternately by 350- and 380-nm light using a Cairn monochromator; emission data were collected at 480-540 nm using a bandpass filter. Experiments were conducted at room temperature with an image collected every 2-3 s. Lysosomal Ca²⁺ content was assessed upon addition of 200 mM GPN (Santa Cruz Biotechnology) which lyses Cathepsin-containing acidic intracellular Ca²⁺ stores. At the end of each run, auto fluorescence was determined by addition of 1 μM ionomycin (Calbiochem) with 4 mM MnCl₂, which quenches fura-2. Images were analysed using custom-written Magipix software (R. Jacob, Kings College London, London, UK) on a single-cell basis, the auto fluorescence signal was subtracted and the data expressed as the maximal peak fluorescence changes (D350/380).

Cathepsin C Activity.

The lysosomes of RAW 264.7 macrophages (that had been infected with BCG_mCherry for 24 hours and those of control cells) were labelled with LysoTracker Green DND-26 (100 nM for 5 min at room temperature) in a buffer containing (mM): 121 NaCl, 5.4 KCl, 0.8 MgCl₂, 1.8 CaCl₂, 6 NaHCO₃, 25 HEPES, 10 Glucose). The cells were washed once in the same buffer but without Ca²⁺ (Ca²⁺-free buffer) and supplemented with 1 mM EGTA. The cells were then washed twice with Ca²⁺-free buffer containing 100 μM EGTA and subsequent experiments conducted in this buffer. The cells were mounted on the stage of a Zeiss LSM510 Meta confocal laser-scanning microscope equipped with a 40× objective; excitation/emission (nm): green (488/505-530), red (543/>560). Experiments were conducted at room temperature with an image collected every 1 s. The activity of Cathepsin C was inferred from the release of LysoTracker (i.e. a decrease in fluorescence) from lysosomes upon the addition of the lysosome-disrupting cathepsin C substrate, Glycyl-L-phenylalanine 2-naphthylamide (GPN, 200 μM). Images were analysed using custom-written Magipix software (R. Jacob, King's College London, London, UK) —on a single-cell basis. Data are presented as the mean±S.E.M. of the initial rate (units of LysoTracker fluorescence per second normalised to the basal fluorescence) or by the rate constant calculated from an exponential curve fit.

Sphingosine HPLC Measurement.

Performed as described (He, X. Et al. Anal Biochem 340, 113-122 (2005).

GSL HPLC Measurement.

Performed as described (Neville, D. C. et al. Anal Biochem 331, 275-282, 2004).

Cholesterol Measurement.

Cholesterol and cholesterol esters were quantified using the Amplex Red Molecular Probes kit, according to manufacturers instructions. Cellular cholesterol was visualised using filipin (Polysciences, Warrington Pa., USA) (Lloyd-Evans, E. et al. Nat Med 14, 1247-1255, doi:nm.1876 [pii]10.1038/nm.1876, 2008).

Sphingomyelin Measurement.

Intracellular sphingomyelin was visualised using Lysenin (Peptides International, Inc., Louisville Ky., USA) as described (Lloyd-Evans, E. et al. Nat Med 14, 1247-1255, doi:nm.1876 [pii]10.1038/nm. 1876, 2008).

Cholera Toxin B Subunit (CtxB) Transport Assays.

Cells were incubated with 0.5 mg/ml Alexa Fluor CtxB for 30 min at room temperature followed by two washes in complete medium and further incubation for 2h at 37° C. in complete medium. Cells were then washed twice in PBS and fixed for 15 min at room temperature with 3.7% paraformaldehyde.

Lysotracker Staining.

Cells were live stained with 50 nM Lysotracker green (Molecular Probes) in PBS at room temperature for 30 min prior to washing.

BODIPY-LacCer Transport.

Performed as described (Lloyd-Evans, E. et al. Nat Med 14, 1247-1255, doi:nm. 1876 [pii] 10.1038/nm. 1876, 2008) with minor modifications.

BODIPY-LacCer was used at a concentration of 5 μM.

Clearance of M. smegmatis

Host cells on coverslips were infected with M. smegmatis (MOI 12.5) and incubated at 37° C./5% C02 for 2h. Cells were washed, and incubated at 37° C./5% CO₂ with fresh medium. At stated time points coverslips were washed, PFA fixed, and M. smegmatis clearance quantified via microscopy.

Treatments and Analysis of Infected Cells.

Cells were infected with BCG 48h prior to washing and addition of therapeutics. Concentrations and duration of treatment were as indicated. Cells were paraformaldehyde fixed (4% PFA 15 minutes room temperature), and levels of fluorescence quantified using fluorescence activated cell sorting (FACS). Samples were acquired on a three laser BD FACS Canto™ II flow cytometer using BD FACSDiva™ software version 6.1 collecting a minimum of 10 000 events.

Electron Microscopy.

Pelleted human macrophages in 1.5 ml PBS were slowly mixed with 25% glutaraldehyde (GA) to achieve a final concentration of 0.1%. The pellet was fixed for 30 min on ice. Pellets washed twice with cacodylate buffer on ice and the pellet fixed in a solution of 2% PFA, 2.5% GA in 0.1M cacodylate buffer on ice for 15 min, and at RT for 2h. Pellets were then washed 3× with 0.1M cacodylate, treated with a solution of 0.1M glycine for 45 min before post fixation in 1% osmium tetroxide for 2h, followed by cacodylate and maleate buffer washes, further post fixation and staining with 1% uranyl acetate in maleate buffer for 1 h in the dark. They were then washed with maleate buffer and dehydrated through an ascending series of ice-cold ethanol up to 100% ethanol, followed by three changes of dry ethanol at room temperature, three changes of propylene oxide, and infiltration with a 1:1 araldite: propylene oxide for 1.5h. Propylene oxide was blown off, pellets embedded in resin and maintained at 60° C. for 3 days prior to sectioning. For Kupffer cell analysis, Npc^{1-/-} mice were PBS perfused, followed by perfusion with a mixture containing 4% PFA, 15% picric acid, and 0.5% GA in 0.1M cacodylate buffer for 20 min. Liver was removed, sectioned, and fixed a solution of 2% PFA, 2.5% GA, 2 mM calcium chloride and 0.1% picric acid in 0.1M cacodylate buffer on ice for 2h, prior to incubation overnight. Samples were then fixed at room temperature for 6h, returned to 4° C. overnight, washed with cacodylate buffer and transferred to antifreeze and allowed to sink overnight. Samples were cut into 1-2 mm cubes in 0.1M cacodylate buffer with 2 mM calcium chloride, washed thrice, and post-fixed for 90 min in 1% osmium tetroxide buffer. From this point protocol was as described above.

Quantification of NPC1/2 Protein Levels.

Proteins were separated on SDS-PAGE and transferred to nitrocellulose membrane (Immobilon P). Membranes were blocked overnight at 4° C. in Tris Buffered Saline (TBS) containing 0.1% Tween-20 and 5% powdered milk, before probing with anti-NPC1 Antibody (Pierce) overnight at 4° C. washed and then probed with Horseradish Peroxidase conjugated anti-rabbit secondary antibody (Pierce). Blots were developed with chemiluminescent substrate (Pierce) and images collected on CCD imager (Biorad). Membranes were stripped and re-probed with anti-β-actin antibody to confirm equal protein loading into each lane.

Statistical Analysis.

All statistical analyses performed with Graphpad Prism.

Results

Infection with Pathogenic Mycobacteria Induce NPC Phenotypes in Murine and Human Macrophages.

NPC cells display a unique combination of cellular phenotypes that include storage of sphingomyelin, GSLs and sphingosine, mistrafficking of GSLs from the Golgi recycling-pathway to LE/Lys (Chen, C. S., Patterson, M. C., Wheatley, C. L., O'Brien, J. F. & Pagano, R. E. Broad screening test for sphingolipid-storage diseases. Lancet 354, 901-905; 1999) and reduced luminal LE/Lys Ca²⁺ storage and impaired LE/Lys Ca2⁺ release (Lloyd-Evans, E. & Platt, F. M. Traffic 11, 419-428, doi:TRA1032 [pii]10.1111/j.1600-0854.2010.01032.x (2010); Lloyd-Evans, E. et al. Nat Med 14, 1247-1255, doi:nm.1876 [pii] 10.1038/nm. 1876 (2008)).

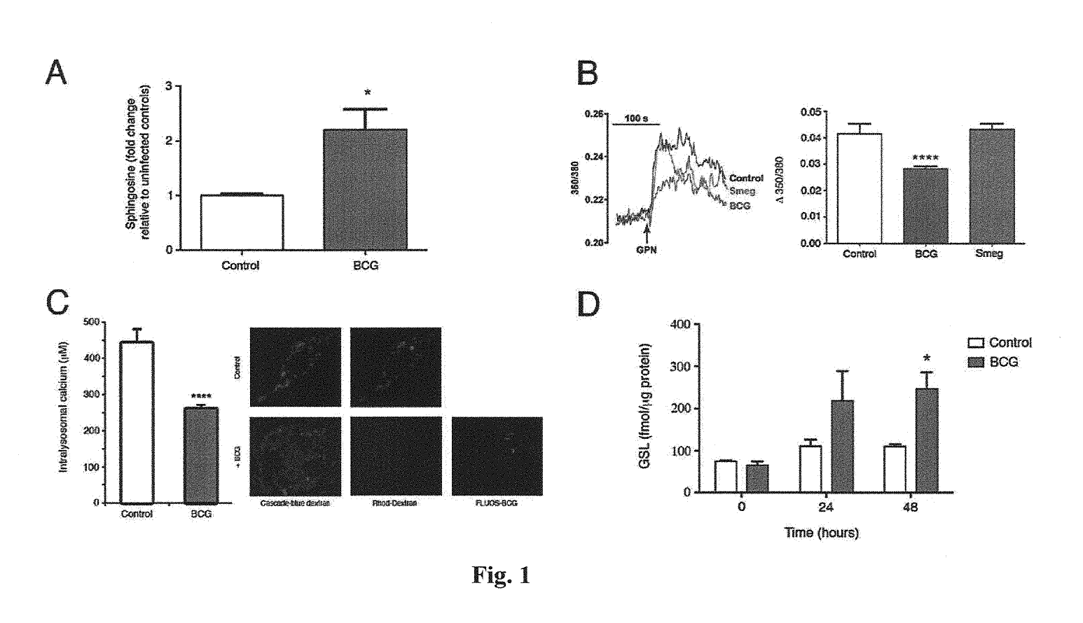

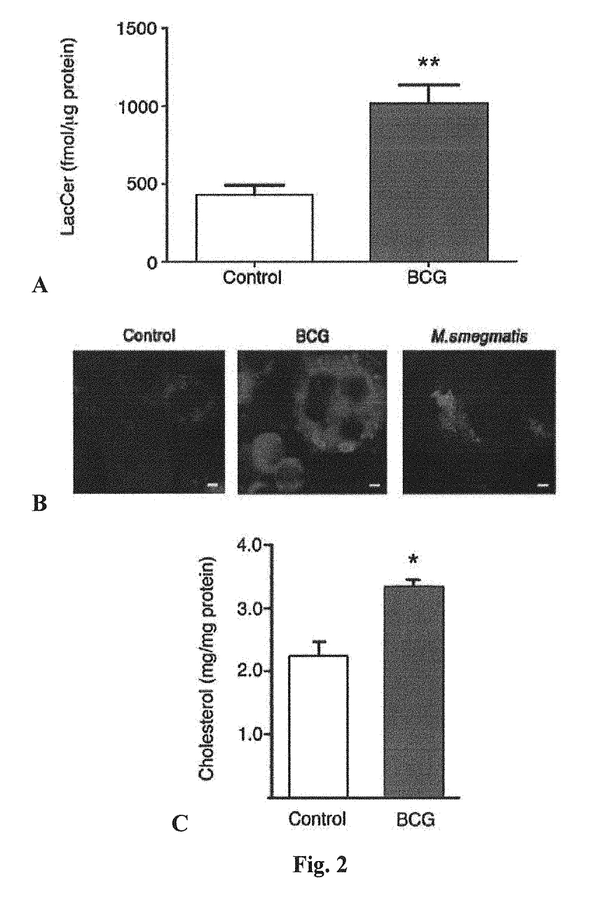

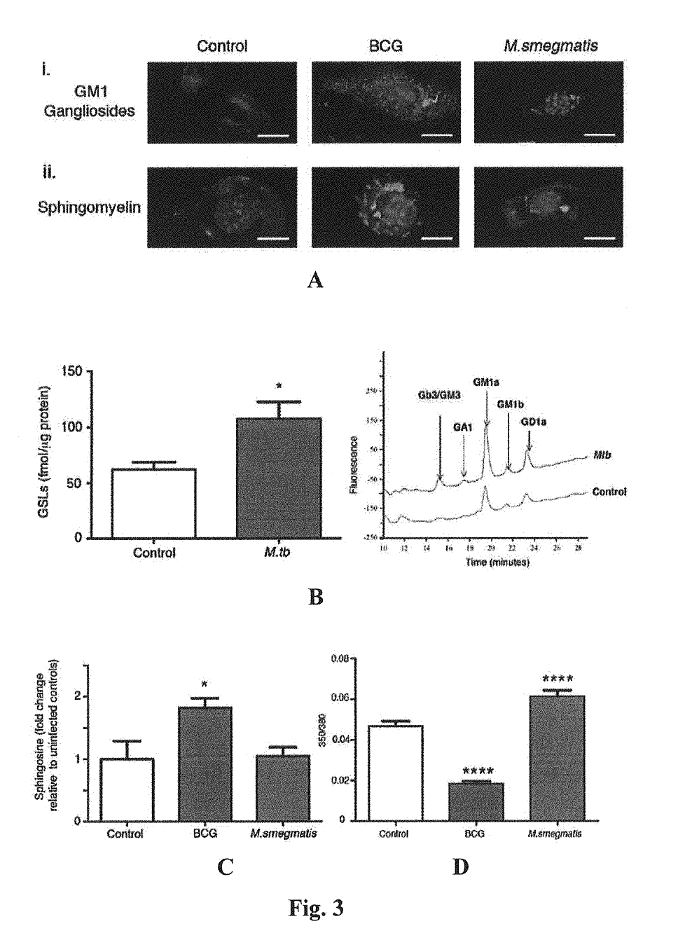

The inventors investigated whether comparable phenotypes occur in cells infected with pathogenic mycobacteria. The murine macrophage cell line RAW 264.7 was infected with live BCG (Pasteur strain), an attenuated form of Mycobacterium bovis, which is a widely studied model of early stage Mtb infection. In agreement with known NPC cellular phenotypes (Lloyd-Evans, E. et al. Nat Med 14, 1247-1255, doi:nm.1876 [pii] 10.1038/nm.1876; 2008), BCG infected macrophages exhibited elevated sphingosine levels relative to uninfected cells 48 hours post-infection (FIG. 1A, p<0.05). Infected macrophages also exhibited reduced LE/Lys Ca²⁺, as assessed indirectly by discharging their Ca²⁺ content with the lysomotropic agent glycyl-L-phenylalanine-β-napthylamide (GPN) (an agent that induces osmotic lysis upon hydrolysis by Cathepsin C) (Lloyd-Evans, E. et al. Nat Med 14, 1247-1255, doi:nm.1876 [pii]10.1038/nm.1876; 2008); a 24-hour infection with BCG was associated with a reduction in GPN-induced Ca²⁺ release, whereas infection with non-pathogenic Mycobacterium smegmatis (M. smegmatis) was not (FIG. 1B, p<0.001). Direct measurement of endo-lysosomal Ca²⁺ content with a luminal Ca²⁺-dye (low-affinity Rhod-dextran) confirmed reduced levels of lysosomal Ca²⁺ in BCG-infected RAW cells (FIG. 1C, p<0.001). Neither cathepsin C activity nor lysosomal pH was affected by BCG infection. Another NPC phenotype is elevated GSLs. Total GSL levels were significantly elevated by 48 hours (FIG. 1D, p<0.05). Accumulation of lactosylceramide (LacCer), which occurs in NPC cells and in tissues of Npc1^{-/-} mice was not detected at 24 and 48 hours post-infection (BCG-infected RAW 264.7 cells) but was significantly elevated 7 days post-infection (FIG. 2A p<0.01). The most widely recognised cellular hallmark of NPC cells is the storage of cholesterol within LE/Lys (Vanier, M. T. Niemann-Pick disease type C. Orphanet J Rare Dis 5, 16, doi:10.1186/1750-1172-5-16 (2010); Vanier, M. T. Lipid changes in Niemann-Pick disease type C brain: personal experience and review of the literature. Neurochem Res 24, 481-489; 1999). When assayed using the fluorescent cholesterol-binding antibiotic, filipin, cholesterol accumulation in punctate structures was observed in BCG infected RAW 264.7 cells, but not in cells infected with non-pathogenic M. smegmatis (FIG. 2B). Biochemical quantitation of cholesterol confirmed higher levels in BCG-infected cells (FIG. 2C, p<0.05). Interestingly, storage of cholesterol was not restricted to cells infected with BCG; neighbouring, uninfected cells also displayed elevated cholesterol storage (FIG. 2B) suggesting local paracrine factors released from infected cells. Other cellular hallmarks of NPC were also induced by BCG infection but not by M. smegmatis. This was demonstrated using fluorescently conjugated cholera toxin subunit B and lysenin that measure the storage and mislocalisation (altered trafficking) of GM1 ganglioside (FIG. 3Ai) and sphingomyelin respectively (FIG. 3AHii).

To determine the relevance of the inventors' findings with BCG to the human pathogen Mtb, the same cell line was infected with live Mtb (H37rv strain). After 48 hours post-infection total cellular GSL levels were measured and confirmed statistically significant elevation of GSLs in Mtb-infected cells (FIG. 3Bi, p<0.0⁵); representative HPLC traces are shown (FIG. 3Bii).

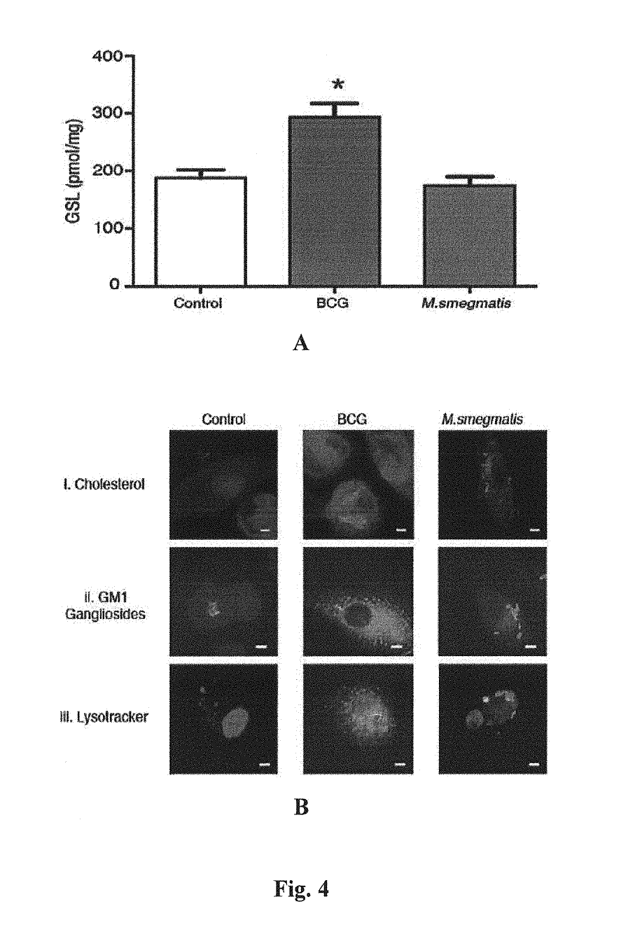

To determine whether the findings in a murine macrophage cell line would be replicated in primary human macrophages, which are more relevant for Mtb infection/TB, monocyte-derived macrophages from healthy donors were infected with BCG. Increased levels of sphingosine (FIG. 3C), reduced LE/Lys Ca²⁺ levels indirectly assayed by GPN-evoked Ca²⁺ release (FIG. 3D) and elevated GSLs (FIG. 4A) were observed. Similarly, cholesterol storage in LE/Lys was also detected in BCG-infected human macrophages (and in non-infected neighbouring cells) (FIG. 4Bi), accompanied by mistrafficking of GM1 ganglioside (FIG. 4Bii). Significant expansion of the lysosomal compartment (a phenotype of lysosomal storage disease cells due to increased lysosomal volume and increased lysosomal biogenesis, visualised with Lysotracker (Lachmann, R. H. et al. Treatment with miglustat reverses the lipid-trafficking defect in Niemann-Pick disease type C. Neurobiol Dis 16, 654-658 (2004); te Vruchte, D. et al. Relative acidic compartment volume as a lysosomal storage disorder-associated biomarker. J Clin Invest 124, 1320-1328, doi:10.1172/JCI72835; 2014) was also detected, another hallmark of NPC (FIG. 4Biii). None of these changes occurred when human macrophages were infected with non-pathogenic M. smegmatis (FIG. 4Bi-iii). Analysis by electron microscopy revealed that whilst control macrophages exhibited normal cellular architecture, BCG-infected cells showed both the presence of intracellular mycobacteria and electron-dense lamellar storage bodies. These were similar to those observed in uninfected Kupffer cells in the liver from the Npc1^{-/-} mouse and in cells with pharmacologically-induced NPC phenotypes (via U18666A treatment). In contrast, cells infected with M. smegmatis exhibited no evidence of storage bodies. Together, these data indicate that pathogenic mycobacteria induce cellular phenotypes indistinguishable from the lysosomal storage disease, NPC.

Mycobacterial Cell Wall Lipids Induce NPC Phenotypes in the Absence of Live Mycobacteria

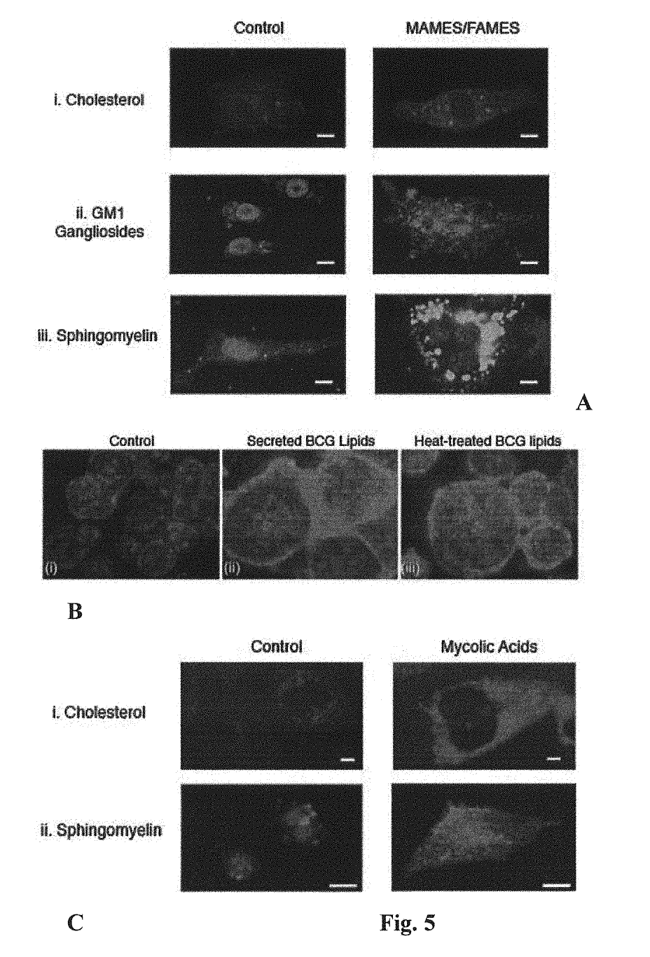

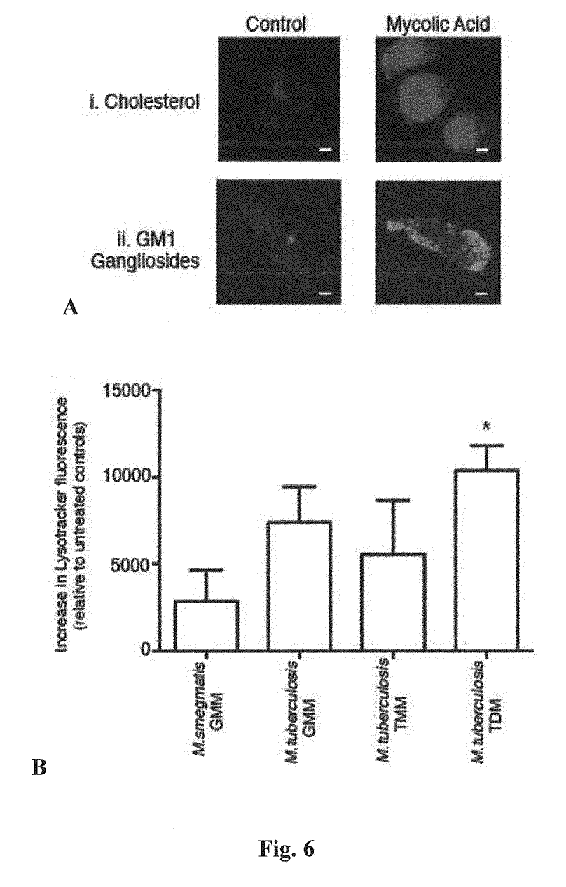

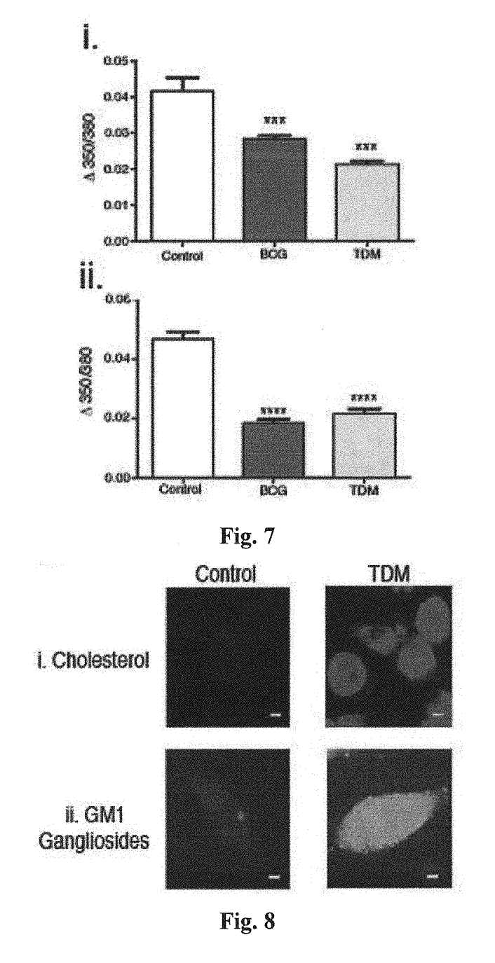

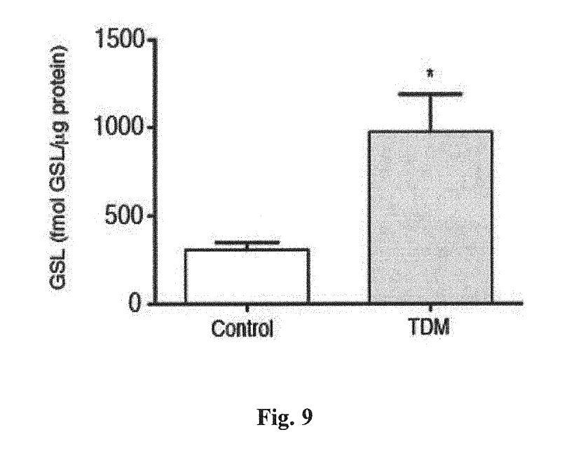

Unexpectedly, cholesterol accumulation was observed in non-infected as well as infected cells (FIGS. 2B and 4Bi). The inventors therefore hypothesised that there is a factor(s) derived from BCG and Mtb that inhibits the NPC pathway and that this is released/secreted and endocytosed by non-infected neighbouring cells. The inventors prepared chloroform: methanol-extracted BCG lipids and found that this fraction induced accumulation/re-distribution of cholesterol (FIG. 5Ai), mistrafficking of GM1 ganglioside (FIG. 5Aii) and accumulation/re-distribution of sphingomyelin (FIG. 5Aiii) in uninfected RAW 264.7 macrophages, comparable to the NPC phenotypes induced by live mycobacteria. The heat-treated preparation had the same ability to affect GM1 ganglioside distribution as the non-heat-treated fraction, suggesting that the NPC phenotype-inducing agent was a lipid and not proteinaceous (FIG. 5B). Mtb-derived mycolic acids (the major cell wall-derived class of lipids in Mtb) also induced NPC phenotypes, including storage of cholesterol and sphingomyelin in RAW 246.7 macrophages, (FIG. 5Ci and ii) and cholesterol storage and GM1 mistrafficking in primary human macrophages (FIG. 6Ai and ii). To narrow down the nature of the causative lipid(s), individual purified mycolic acid ester species derived from Mtb that included trehalose dimycolate (TDM), trehalose monomycolate (TMM) and glucose monomycolate (GMM) were assayed. It was observed that TDM gave the greatest increase in Lysotracker fluorescence, indicative of NPC-like lysosomal expansion/storage (FIG. 6B, p<0.05). The other lipids were less effective: GMM and TMM lipids from Mtb caused only modest lysosomal expansion that did not reach statistical significance, whilst GMM from non-pathogenic M. smegmatis had minimal effects (FIG. 6B). In addition to affecting lysosomal morphology, TDM from M. bovis also recapitulated NPC phenotypes in terms of reduced LE/Lys Ca²⁺ levels (FIG. 7i and ii respectively), cholesterol storage and GM1 mistrafficking (FIG. 8i and ii respectively) and GSL accumulation (FIG. 9). Taken together, the Mtb lipid TDM reproduces the NPC phenotype caused by pathogenic mycobacteria.

Mycobacteria Target the NPC1 Protein

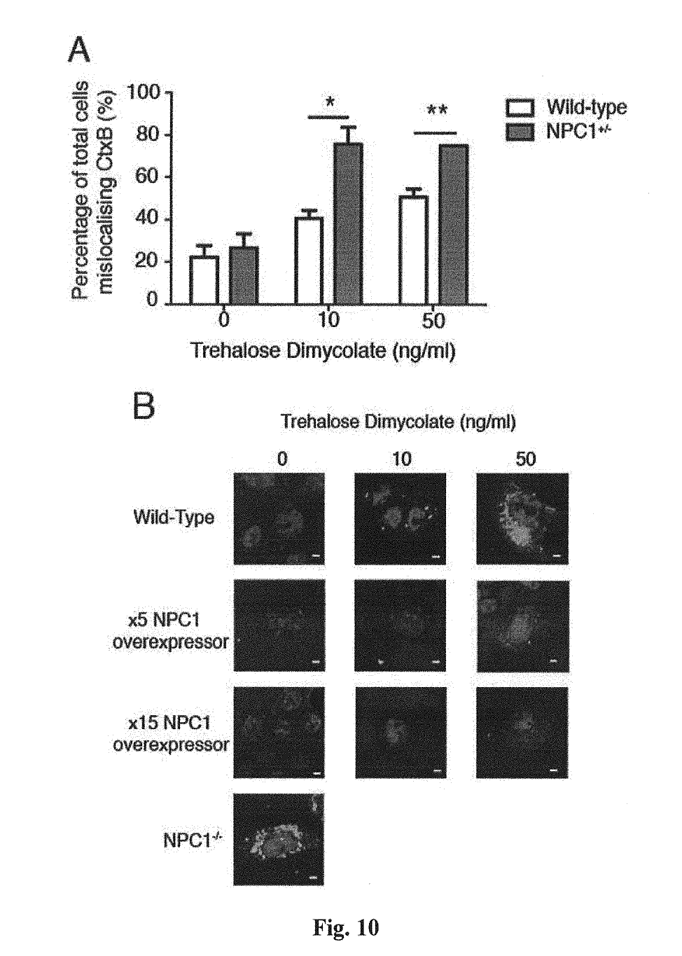

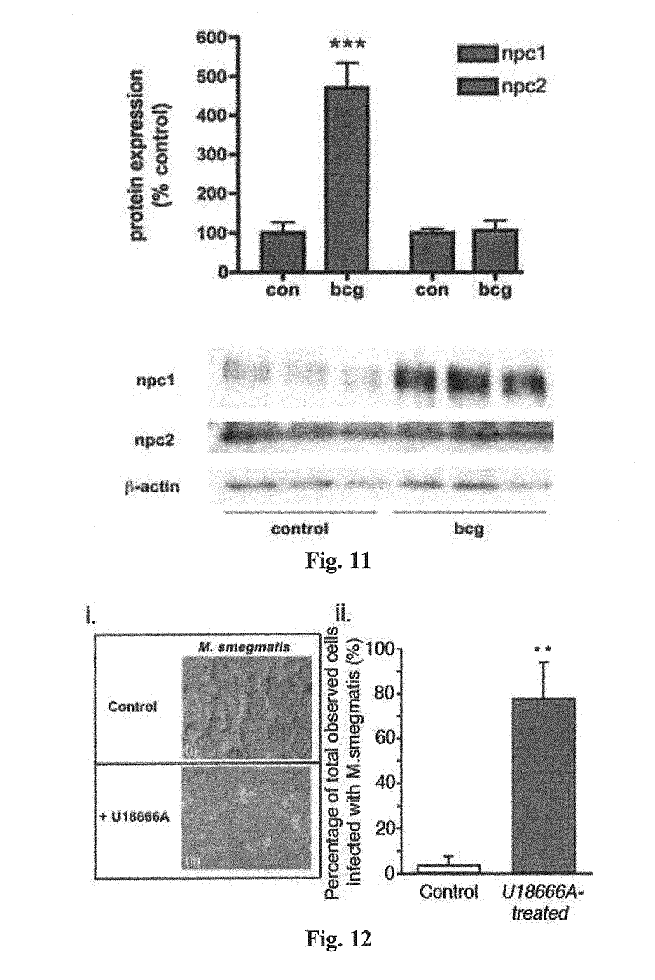

If TDM inhibited the NPC pathway via interaction with the NPC1 protein then the inventors reasoned that NPC1 mice lacking one NPC1 allele (heterozygous NPC1 cells) would be more susceptible to inhibition than wild-type cells. Bone marrow-derived macrophages generated from NPC1 heterozygous mice (Npc1^{+/-}) were therefore incubated with TDM. It was observed that cells with 50% of wild-type NPC1 protein levels were indeed more susceptible to inhibition (FIG. 10A). These data were complemented by the demonstration that CHO cells overexpressing NPC1 were more resistant to induction of NPC cellular phenotypes than wild-type cells when exposed to TDM. The extent of NPC1 overexpression was proportional to the resistance to phenotype induction; cells over-expressing NPC1 by 15-fold were more resistant than those over-expressing NPC1 5-fold (FIG. 10B). To investigate whether the mycobacterial lipids potentially interact with NPC1 or NPC2 proteins, the proteins' expression levels in RAW264.7 cells infected with BCG were examined. Whereas expression of NPC1 protein was up regulated in infected cells, there were no changes in NPC2 levels (FIG. 11).

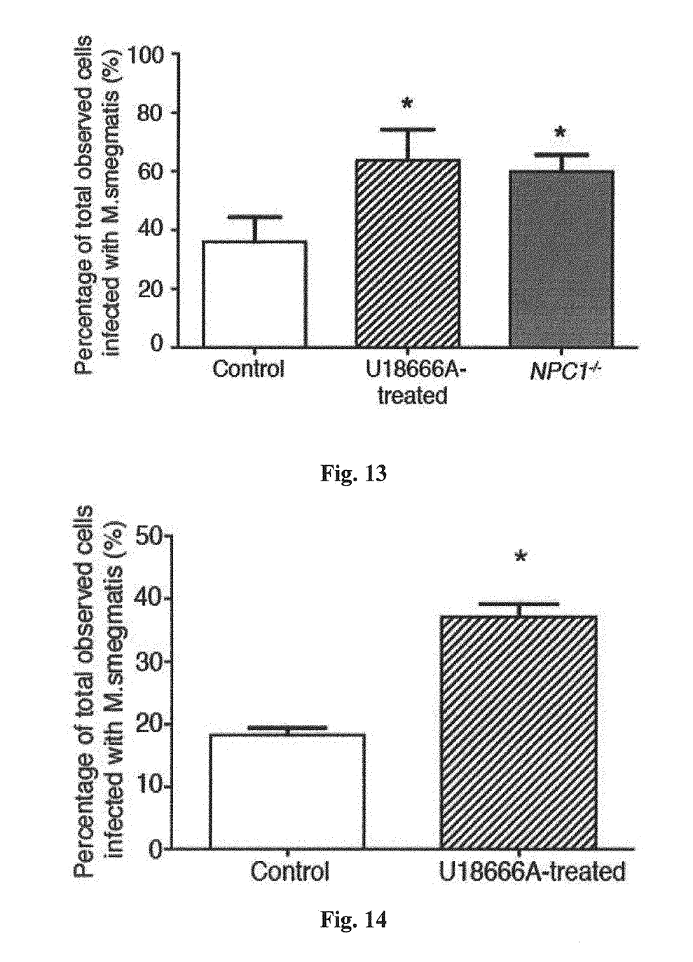

One prediction that can be made from these findings is that NPC patient cells that lack functional NPC1 due to a genetic defect will not be able to clear even non-pathogenic mycobacteria as they have impaired LE/Lys fusion. Non-pathogenic mycobacterial species normally cannot inhibit phagosome-lysosome fusion (they may lack inhibitory lipids) and are readily cleared by healthy cells. Consistent with this hypothesis, RAW 246.7 cells in which an NPC phenotype was pharmacologically induced by incubation with U18666A, prior to infection, had impaired ability to clear non-pathogenic M. smegmatis (FIG. 12) relative to untreated cells. Impaired clearance of M. smegmatis was also observed in Npc1^{-/-} bone marrow-derived mouse macrophages, U18666A-treated wild-type mouse macrophages (FIG. 13) and primary human macrophages treated with U18666A (FIG. 14). This has important implications for NPC disease patients as they may have altered microbial handling and an altered microbiome.

NPC Therapeutics Promote Clearance of Pathogenic Mycobacteria Via a Ca²⁺-Dependent Mechanism.

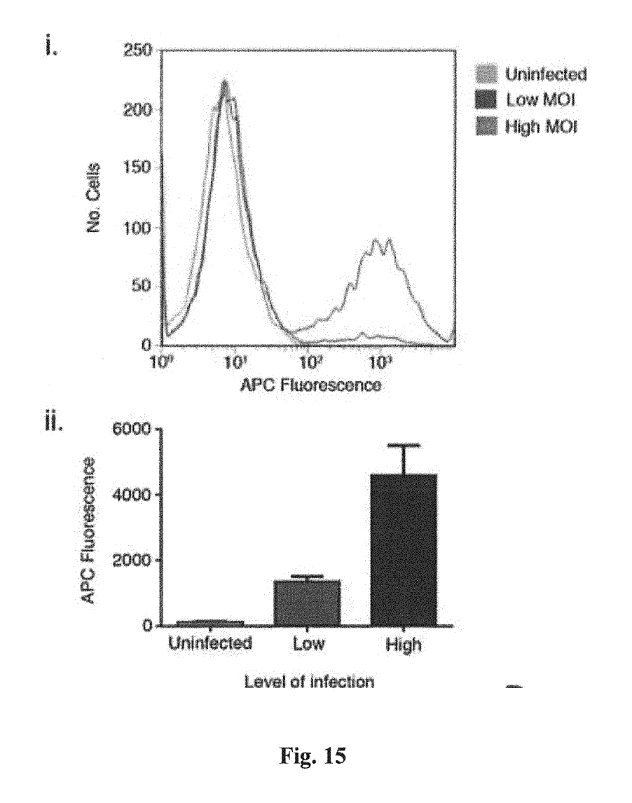

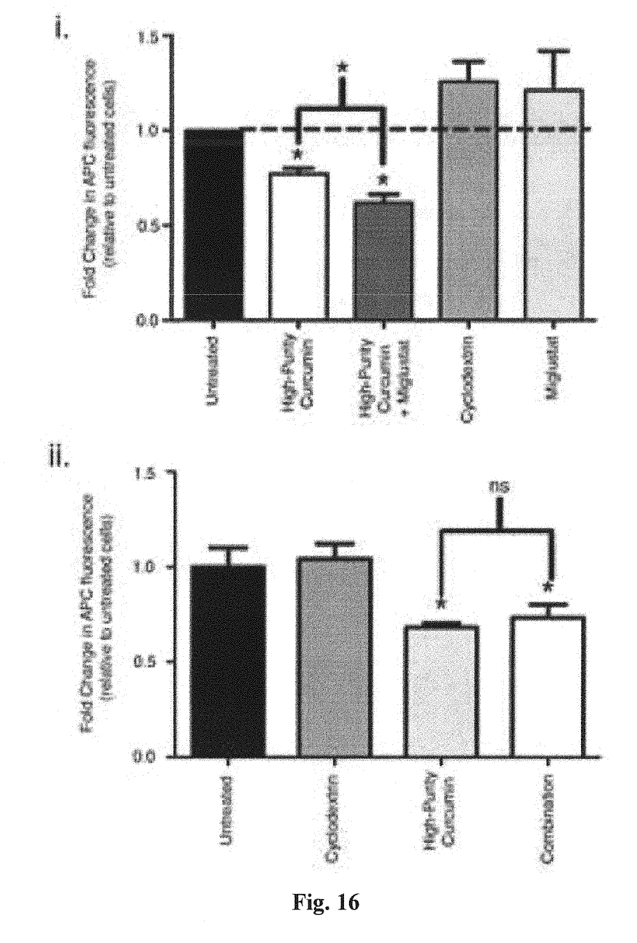

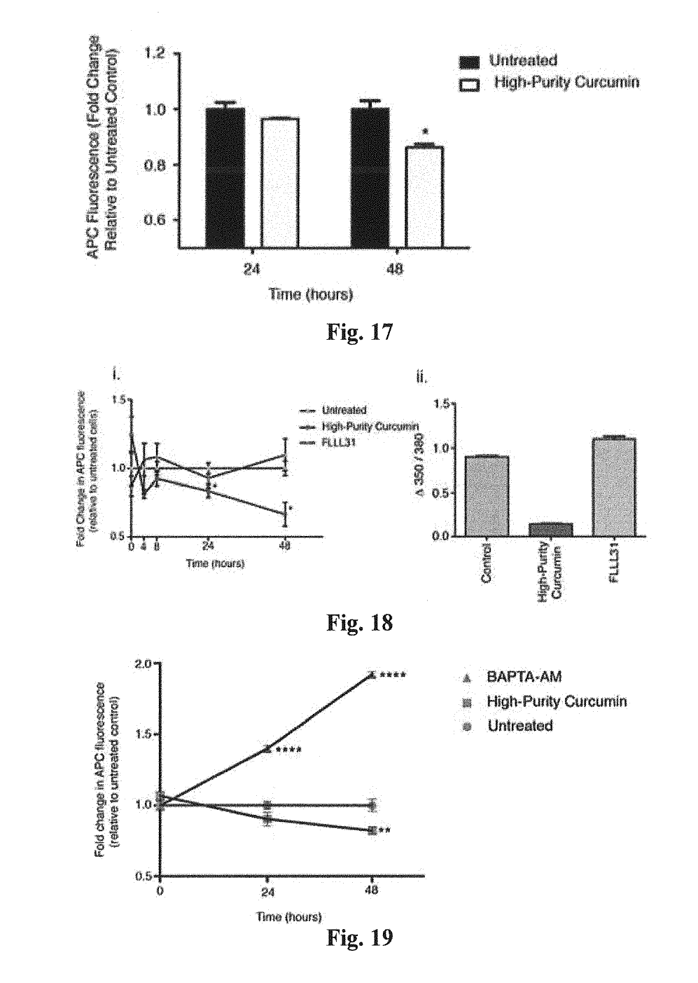

Transport and fusion events in the late endocytic system are critically dependent upon Ca²⁺ release from LE/Lys (Lloyd-Evans, E. et al. Nat Med 14, 1247-1255, doi:nm.1876 [pii] 10.1038/nm. 1876; 2008). This release is impaired in NPC, thereby preventing organelle fusion (Lloyd-Evans, E. et al. Nat Med 14, 1247-1255, doi:nm.1876 [pii] 10.1038/nm.1876; 2008). Nonetheless, elevation of cytosolic Ca²⁺ by pharmacologically manipulating other Ca²⁺ stores (such as the ER) can compensate for the defect in LE/Lys Ca²⁺ release and correct the NPC phenotypes (Lloyd-Evans, E. et al. Nat Med 14, 1247-1255, doi:nm.1876 [pii]10.1038/nm.1876; 2008). Curcumin inhibits Ca²⁺ uptake by the sarco-endoplasmic reticulum Ca²⁺-ATPase (SERCA) (Bilmen, J. G., et al. Eur J Biochem 268, 6318-6327, doi:2589 [pii]; 2001) and thereby causes Ca²⁺ leak from the ER. The subsequent elevated of cytosolic [Ca²⁺ ] overcomes the block in LE/Lys fusion and rescues NPC phenotypes (Lloyd-Evans, E. et al. Nat Med 14, 1247-1255, doi:nm.1876 [pii] 10.1038/nm. 1876; 2008). It was therefore tested whether curcumin promotes clearance of pathogenic mycobacteria. RAW264.7 cells were infected with mCherry-expressing BCG for 48 hours, then treated with or without 30 μM high-purity curcumin for a further 24 hours. A FACS assay was used to determine the frequency and extent of mycobacterial infection by quantifying the fluorescence of mCherry-expressing M. bovis BCG (FIG. 15). The average fluorescence intensity indicated a significantly lower level of infection (i.e. enhanced clearance) in cells incubated with curcumin relative to untreated cells (FIG. 16, p<0.05). The capacity of two other compounds that have shown efficacy in NPC cells was tested; miglustat, an imino sugar inhibitor of GSL biosynthesis (EMA approved for NPC therapy (Patterson, M. C. et al. Rev Neurol (separata) 43, 8, 2006; Pineda, M. et al. Mol Genet Metab 98, 243-249, doi:10.1016/j.ymgme.2009.07.003, 2009) and 3-cyclodextrin, a cyclic oligosaccharide that has shown efficacy in animal models of NPC (Davidson, C. D. et al. PLoS One 4, e6951, doi:10.1371/journal.pone.0006951; 2009; Ramirez, C. M. et al. Pediatr Res 68, 309-315, doi:10.1203/PDR.Ob013e3181ee4dd2; 2010; Camargo, F. et al. Life Sci 70, 131-142, 2001; Stein, V. M. et al. J Neuropathol Exp Neurol 71, 434-448, doi: 10.1097/NEN.0b013e31825414a6, 2012). Although neither of these molecules alone promoted mycobacterial clearance over this time course (FIG. 16), miglustat in combination with curcumin showed synergy. No such synergy was seen when curcumin and cyclodextrin were used in combination. Curcumin also significantly reduced mycobacterial burden in infected primary human macrophages (FIG. 17, p<0.05). Several pieces of evidence suggest that the enhancement of BCG clearance by curcumin was dependent upon its ER Ca²⁺-mobilising properties, assessed by probing ER/neutral Ca²⁺ store Ca²⁺ content with ionomycin. Whilst high-purity curcumin (SERCA antagonist) promoted BCG clearance, the inactive curcumin analogue FLLL31 (not a SERCA antagonist) had no effect (FIG. 18). Furthermore, bacterial clearance by curcumin, was abrogated by loading cells with the Ca²⁺ chelator, BAPTA, to suppress a Ca²⁺ increase (FIG. 19). Although curcuminoids have previously been reported to have direct anti-Mtb activity in host-cell free systems, the slow kinetics of such an anti-bacterial action cannot account for the relatively rapid effects that the inventors observed: it took >4 days for high-purity curcumin to reduce BCG growth in broth. In view of the differences in time scale, it seems likely that the enhanced clearance observed here (FIGS. 16-19) results from curcumin modulating intracellular Ca^{2+.}

Discussion

Through these investigations into the pathogenesis of NPC it was unexpectedly found that Mtb inhibits the NPC pathway, promoting its intra-cellular survival in host macrophages. This link between this rare lysosomal storage disorder and Mtb infection has several important implications for understanding host-pathogen interactions and for developing new therapies to combat TB, particularly in the current era of antibiotic resistance.

Phagocytosed Mtb bacilli undergo a period of rapid multiplication, concomitant with granuloma development (Russell, D. G., et al. Nat Immunol 10, 943-948, doi:10.1038/ni.1781; 2009). A significant element of the mycobacterial intracellular survival strategy is its ability to inhibit phagosome-lysosome fusion, thereby avoiding lysosomal destruction. Here, evidence is provided supporting a model in which pathogenic mycobacteria, such as Mtb and BCG, secrete lipids that inhibit the NPC pathway, phenocopying NPC1^{-/-} cells. The NPC phenotypes induced in host cells include elevated levels of sphingosine, which in turn inhibits LE/Lys Ca² store filling (Lloyd-Evans, E. et al. Nat Med 14, 1247-1255, doi:nm.1876 [pii]10.1038/nm.1876; 2008), leading to reduce phagosome-lysosome fusion facilitating intracellular mycobacterial survival. Pharmacological compensation for this Ca²⁺ homeostatic defect by decreasing Ca²⁺ buffering by the ER, presumably in the vicinity of lysosomes, enhanced clearance of pathogenic mycobacteria, offering a new approach to potentially treat latent Mtb infection. These findings also contribute to the debate on the involvement of Ca²⁺ in phagosome-lysosome fusion and support published studies suggesting it is a Ca²⁺ dependent process (Majeed, M., et al. Microbial pathogenesis 24, 309-320, doi:10.1006/mpat.1997.0200, 1998).

Interestingly, the inventors observed that induction of NPC phenotypes was not restricted to macrophages that harbour internalised mycobacteria, but was also observed in uninfected bystander cells. Cell wall-derived lipids from pathogenic mycobacteria are actively trafficked out of the phagosome and distributed within the infected cell, as well as within extracellular vesicles that can be endocytosed by bystander macrophages (Beatty, W. L. et al. Traffic 1, 235-247, 2000). The inventors found that exposure to a lipid fraction from BCG and mycolic acids derived from Mtb both induced NPC cellular phenotypes, replicating the effect of the intact mycobacterium. Of the structurally distinct lipid classes tested the TDM fraction gave the largest increase in lysosomal expansion. The immunomodulatory properties of TDM (also known as cord factor) have been previously documented, with it initiating pro-inflammatory responses (Bowdish, D. M. et al. PLoS Pathog 5, e1000474, doi:10.1371/journal.ppat.1000474, 2009) and most significantly can induce granuloma formation in mice in the absence of the intact mycobacterium (Kim, M. J. et al. EMBO molecular medicine 2, 258-274, doi:10.1002/emmm.201000079, 2010). This lipid species therefore has the capability to induce multiple responses in the infected host cell, many of which are likely to be beneficial for its survival. The importance of TDM is further reinforced by the fact that mycobacteria possessing lower levels of this lipid (either due to mutation or chemical removal) have reduced virulence and an impaired capacity to modulate endocytic trafficking and phagosome maturation (Katti, M. K. et al. Cellular microbiology 10, 1286-1303, doi:10.1111/j.1462-5822.2008.01126.x, 2008; Indrigo, J., et al. Microbiology 149, 2049-2059, 2003).

The simplest hypothesis to explain the inventors' findings is that TDM lipids directly bind to NPC1 and inhibit its function, although an indirect mechanism dependent on NPC1 cannot be ruled out. There is currently no structure for intact NPC1 and no direct functional assay for NPC1, making this a technically difficult hypothesis to test. NPC1 is a mammalian orthologue of an ancient family of bacterial transporters termed Root Nodulation Division (RND) permeases (Davies, J. P., Science 290, 2295-2298, doi:10.1126/science.290.5500.2295 290/5500/2295 [pii], 2000). Interestingly, members of this family of proteins (termed MmpL) act as mycolic acid transporters, facilitating lipid secretion by mycobacteria (including Mtb) (Varela, C. et al. Chemistry & biology 19, 498-506, doi: 10.1016/j.chembiol.2012.03.006, 2012). Indeed, SQ109 a drug that targets this transporter is currently in clinical trials for treating tuberculosis (Tahlan, K. et al. Antimicrob Agents Chemother 56, 1797-1809, doi:10.1128/AAC.05708-11, 2012). This implies that members of this conserved family of RND proteins have the ability to bind mycolic acids, particularly TMM (Varela, C. et al. Chemistry & biology 19, 498-506, doi:10.1016/j.chembiol.2012.03.006, 2012) and indeed Mmpl3 is essential for viability of Mtb (Domenech, P., et al., Infection and immunity 73, 3492-3501, doi: 10.1128/IAI.73.6.3492-3501.2005, 2005). It may therefore be the case that the mammalian NPC1 protein also binds mycolic acids, but with the lipid acting as an inhibitor not a substrate. Taken together, these studies demonstrate a remarkable role for mycobacterial RND permease family members. They are essential virulence factors for pathogen survival where they serve as mycolic acid transporters, but their mammalian counterpart NPC1 is also be targeted by the pathogen once within the host cell. The complex biology of the RND permease family of proteins remains incompletely understood and merits further investigation.

In this study, the inventors provide several lines of evidence supporting NPC1 as the potential target for inhibitory lipids shed by pathogenic mycobacteria. The susceptibility of cells to the induction of NPC phenotypes was dependent upon the relative levels of the NPC1 protein; Npc1^{+/-} macrophages were more sensitive than wild type whilst overexpression conferred resistance. NPC1, but not NPC2, was significantly up regulated in BCG infected macrophages; significantly the NPC1 protein is also up regulated in Mtb granulomas in vivo (Kim, M. J. et al. EMBO molecular medicine 2, 258-274, doi:10.1002/emmm.201000079, 2010), perhaps reflecting an attempt to compensate for reduced function. This upregulation may act to slow the rate of induction of NPC disease cellular phenotypes by the mycobacterium as the inventors saw in the over NPC1 overexpressing cells. However, the enhanced copy number of NPC1 protein will still be subject to inhibition by mycobacterial lipids, so cannot prevent the development of stable infection over time. Finally, pharmacological or genetic blockade of NPC1 significantly enhanced the survival of non-pathogenic mycobacterial species. This may have significant implications for NPC patients as it would suggest that they are likely to have altered microbial handling, and as a result harbour an unusual microbiome, and potentially have greater susceptibly to Mtb infection.

The inventors reasoned that should inhibition of the NPC pathway be central to the intracellular survival of pathogenic mycobacteria, pharmacological agents that correct NPC cells should promote clearance of the mycobacterium. Consistent with this hypothesis, a significantly lower bacterial load was measured in cells treated with curcumin, a natural product that raises cytosolic [Ca²⁺ ], but not in those exposed to the curcumin analogue (FLLL31) that has no effect on cytosolic Ca²⁺ levels. The inventors investigated whether two other drugs that have shown efficacy in NPC might also promote elimination of mycobacteria. However, enhanced microbial clearance was not detected when either β-cyclodextrin (which can ameliorate disease symptoms in animal models (Davidson, C. D. et al. PLoS One 4, e6951, doi:10.1371/journal.pone.0006951, 2009) possibly via stimulation of exocytosis (Chen, F. W., et al., PLoS One 5, doi:e15054 10.1371/journal.pone.0015054, 2010)) or miglustat (a GSL biosynthesis inhibitor (Platt, F. M., et al., J Biol Chem 269, 8362-8365, 1994) clinically approved for NPC (Patterson, M. C., et al., Lancet neurology 6, 765-772, 2007; Wraith, J. E. et al., Mol Genet Metab 99, 351-357, doi: 10.1016/j.ymgme.2009.12.006, 2010)) were tested. Interestingly, miglustat showed synergy when combined with curcumin. The lack of effect with cyclodextrin would support the proposed exocytotic mechanism of action in NPC (Chen, F. W., et al., PLoS One 5, doi:e15054 10.1371/journal.pone.0015054, 2010), which would not affect the phagosome.

In summary, the inventors have demonstrated for the first time a mechanistic relationship between a rare, inherited fatal neurodegenerative lysosomal disorder and the process used by the most successful human intracellular pathogen to subvert cellular defences. These findings provide not only an explanation for the defective phagosomal maturation observed following Mtb infection, but also provide a unified mechanistic framework accounting for other unexplained phenotypes in Mtb infected macrophages, including cholesterol (Peyron, P. et al. PLoS Pathog 4, e1000204, doi:10.1371/journal.ppat.1000204, 2008) and LacCer storage (Kim, M. J. et al. EMBO molecular medicine 2, 258-274, doi:10.1002/emmm.201000079, 2010), calcium homeostatic defects (Majeed, M., et al. Microbial pathogenesis 24, 309-320, doi:10.1006/mpat.1997.0200, 1998), elevated NPC1 expression (Kim, M. J. et al. EMBO molecular medicine 2, 258-274, doi:10.1002/emmm.201000079, 2010) and bystander effects on neighbouring cells (Beatty, W. L. et al. Traffic 1, 235-247 (2000). These findings also suggest that correcting or compensating for reduced NPC1 function will offer a novel therapeutic approach for treating tuberculosis that targets the host cell and should not be subject to development of resistance.

Although inhibitors of sphingolipid biosynthesis and β-cyclodextrin were found to be inactive, in that neither class of agent promoted mycobacterial clearance when used alone over the time course used, it was unexpectedly found that inhibitors of sphingolipid biosynthesis show synergy when combined with compounds that elevate cytosolic calcium. Indeed, the combination of curcumin, a compound which elevates cytosolic calcium, and miglustat, an inhibitor of sphingolipid biosynthesis, showed significantly greater efficacy than the calcium modulator on its own and, of course, than the inhibitor of sphingolipid biosynthesis on its own which showed no activity. The invention therefore provides a surprisingly efficacious combination therapy for treating infections by pathogenic mycobacteria, and in particular infections by Mtb, which employs a compound that elevates cytosolic calcium in combination with an inhibitor of sphingolipid biosynthesis.

### Example 2—Tablet Composition

Tablets, each weighing 0.15 g and containing 12.5 mg of a compound which elevates cytosolic calcium and 12.5 mg of an inhibitor of sphingolipid biosynthesis, for use in accordance with the invention, are manufactured as follows:

Composition for 10,000 tablets

Active compounds (250 g)

Lactose (800 g)

Corn starch (415 g)

Talc powder (30 g)

Magnesium stearate (5 g)

The active compounds (a compound which elevates cytosolic calcium and an inhibitor of sphingolipid biosynthesis), lactose and half of the corn starch are mixed. The mixture is then forced through a sieve 0.5 mm mesh size. Corn starch (10 g) is suspended in warm water (90 ml). The resulting paste is used to granulate the powder. The granulate is dried and broken up into small fragments on a sieve of 1.4 mm mesh size. The remaining quantity of starch, talc and magnesium is added, carefully mixed and processed into tablets.

### Example 3—Injectable Formulation

Formulation A

| | |
|---|---|
| Active compound 1 | 100 mg |
| Active compound 2 | 100 mg |
| Hydrochloric Acid Solution 0.1M or | 4.0 to 7.0 |
| Sodium Hydroxide Solution 0.1M q.s. to pH | |
| Sterile water q.s. to | 10 ml |

The two active compounds: a compound which elevates cytosolic calcium and an inhibitor of sphingolipid biosynthesis, for use in accordance with the invention, are dissolved in most of the water (35° 40° C.) and the pH adjusted to between 4.0 and 7.0 with the hydrochloric acid or the sodium hydroxide as appropriate. The batch is then made up to volume with water and filtered through a sterile micropore filter into a sterile 10 ml amber glass vial (type 1) and sealed with sterile closures and overseals.

Formulation B

| | |
|---|---|
| Active Compound 1 | 62.5 mg |
| Active Compound 2 | 62.5 mg |
| Sterile, Pyrogen-free, pH 7 Phosphate | 25 ml |
| Buffer, q.s. to | |
| Active compound | 200 mg |
| Benzyl Alcohol | 0.10 g |
| Glycofurol 75 | 1.45 g |
| Water for injection q.s to | 3.00 ml |

The two active compounds: a compound which elevates cytosolic calcium and an inhibitor of sphingolipid biosynthesis, for use in accordance with the invention, are dissolved in the glycofurol. The benzyl alcohol is then added and dissolved, and water added to 3 ml. The mixture is then filtered through a sterile micropore filter and sealed in sterile 3 ml glass vials (type 1).

### Example 4—Syrup Formulation

Active compound 1: 125 mg

Active compound 2: 125 mg

Sorbitol Solution 1.50 g

Glycerol 2.00 g

Sodium benzoate 0.005 g

Flavour 0.0125 ml

Purified Water q.s. to 5.00 ml

The two active compounds: a compound which elevates cytosolic calcium and an inhibitor of sphingolipid biosynthesis, for use in accordance with the invention, are dissolved in a mixture of the glycerol and most of the purified water. An aqueous solution of the sodium benzoate is then added to the solution, followed by addition of the sorbitol solution and finally the flavour. The volume is made up with purified water and mixed well.

1. A method of treatment of an infection by a pathogenic mycobacterium, which method comprises administering to a human or animal patient in need of such treatment an effective amount of a SERCA antagonist and an effective amount of an inhibitor of glycosphingolipid biosynthesis.

2. A method according to claim 1 wherein the infection by a pathogenic mycobacterium is an infection caused by Mycobacterium tuberculosis, Mycobacterium bovis, Mycobacterium africanum, Mycobacterium cannetti or Mycobacterium microtti.

3. A method according to claim 1 wherein the infection by a pathogenic mycobacterium is an infection caused by Mycobacterium tuberculosis.

4. A method according to claim 1 wherein the infection is tuberculosis (TB).

5. A method according to claim 1 wherein the SERCA antagonist is selected from: curcumin, thapsigargin, nortrilobolide and cyclopiazonic acid, and pharmaceutically acceptable salts thereof; and the inhibitor of glycosphingolipid biosynthesis is selected from an iminosugar, D,L-threo-1-phenyl-2-decanoylamino-3-morpholino-1-propanol (PDMP); D,L-threo-1-phenyl-2-hexadecanoylamino-3-morpholino-1-propanol (PPMP); D-threo-1-phenyl-2-palmitoilamino-3-pyrrolidino-1-propanol (P4); 4′-hydroxy-D-threo-1-phenyl-2-palmitoilamino-3-pyrrolidino-1-propanol (4′-hydroxy-P4); 3′,4′-ethylenedioxy-P4 (EtDO-P4); N-[(1R,2R)-1-(2,3-Dihydro-1,4-benzodioxin-6-yl)-1-hydroxy-3-(1-pyrrolidinyl)-2-propanyl]octanamide (eliglustat); and 2,5-dihydroxymethyl-3,4-dihydroxypyrrolidine (L-DMDP), and pharmaceutically acceptable salts thereof.

6. A method according to claim 1 wherein the SERCA antagonist is selected from curcumin, thapsigargin and cyclopiazonic acid, and pharmaceutically acceptable salts thereof; and the inhibitor of glycosphingolipid biosynthesis is an iminosugar or a pharmaceutically acceptable salt of an iminosugar.

7. A method according to claim 1 wherein the SERCA antagonist is curcumin or a pharmaceutically acceptable salt thereof; and the inhibitor of glycosphingolipid biosynthesis is an iminosugar or a pharmaceutically acceptable salt of an iminosugar.

8. A method according to claim 6 wherein the iminosugar is N-butyldeoxynojirimycin (NB-DNJ), N-butyldeoxygalactonojirimycin (NB-DGJ), or a pharmaceutically acceptable salt of NB-DNJ or NB-DGJ.

9. A method according to claim 1 wherein the SERCA antagonist is curcumin or a pharmaceutically acceptable salt thereof; and the inhibitor of glycosphingolipid biosynthesis is N-butyldeoxynojirimycin (NB-DNJ) or a pharmaceutically acceptable salt thereof.

10. A method according to claim 1 wherein the SERCA antagonist is curcumin or a pharmaceutically acceptable salt thereof; and the inhibitor of glycosphingolipid biosynthesis is N-butyldeoxygalactonojirimycin (NB-DGJ) or a pharmaceutically acceptable salt thereof.

11. A method according to claim 1 wherein the SERCA antagonist is curcumin or a pharmaceutically acceptable salt thereof; and the inhibitor of glycosphingolipid biosynthesis is N-[(1R,2R)-1-(2,3-Dihydro-1,4-benzodioxin-6-yl)-1-hydroxy-3-(1-pyrrolidinyl)-2-propanyl]octanamide (eliglustat) or a pharmaceutically acceptable salt of eliglustat.

12. A method according to claim 11 wherein the SERCA antagonist is curcumin or a pharmaceutically acceptable salt thereof; and the inhibitor of glycosphingolipid biosynthesis is eliglustat tartrate.

13-22. (canceled)

23. A method according to claim 1 wherein the infection by a pathogenic mycobacterium is an infection caused by Mycobacterium tuberculosis, the SERCA antagonist is curcumin or a pharmaceutically acceptable salt thereof, and the inhibitor of glycosphingolipid biosynthesis is N-butyldeoxynojirimycin (NB-DNJ) or a pharmaceutically acceptable salt thereof.

24. A method according to claim 1 wherein the SERCA antagonist and the inhibitor of glycosphingolipid biosynthesis are administered to the patient together in the same pharmaceutical composition.

25. A method according to claim 1 wherein the SERCA antagonist and the inhibitor of glycosphingolipid biosynthesis are administered to the patient in different pharmaceutical compositions.

26. A method according to claim 25, wherein the different pharmaceutical compositions are administered separately, simultaneously, concomitantly or sequentially.

27. A method according to claim 1 wherein the patient is human.

28. A kit of parts comprising a SERCA antagonist together with instructions for simultaneous, concurrent, separate or sequential use in combination with an inhibitor of glycosphingolipid biosynthesis, for the treatment of a human or animal patient suffering from or susceptible to an infection by a pathogenic mycobacterium.

29. A kit of parts comprising an inhibitor of glycosphingolipid biosynthesis together with instructions for simultaneous, concurrent, separate or sequential use in combination with a SERCA antagonist, for the treatment of a human or animal patient suffering from or susceptible to an infection by a pathogenic mycobacterium.

30. A kit of parts comprising an inhibitor of glycosphingolipid biosynthesis, a SERCA antagonist, and instructions for their simultaneous, concurrent, separate or sequential use for the treatment of a human or animal patient suffering from or susceptible to an infection by a pathogenic mycobacterium.

31. A kit of parts according to claim 30 wherein the infection by a pathogenic mycobacterium is an infection caused by Mycobacterium tuberculosis, the SERCA antagonist is curcumin or a pharmaceutically acceptable salt thereof, and the inhibitor of glycosphingolipid biosynthesis is N-butyldeoxynojirimycin or a pharmaceutically acceptable salt thereof.

32. A kit of parts according to claim 31 wherein the patient is human.
